(19) United States
(12) Patent Application Publication
(10) Pub. No.: US 2023/0102974 A1
(43) Pub. Date: Mar. 30, 2023

(54) LIGHT-EMITTING DEVICE AND ELECTRONIC APPARATUS INCLUDING THE SAME

(71) Applicant: Samsung Display Co., Ltd., Yongin-si (KR)

(72) Inventors: Namsu Kang, Yongin-si (KR); Songeun Lee, Yongin-si (KR); Heungsu Park, Yongin-si (KR); Huiju Park, Yongin-si (KR); Hyunshik Lee, Yongin-si (KR); Wonsuk Han, Yongin-si (KR)

(21) Appl. No.: 17/645,942

(22) Filed: Dec. 23, 2021

(30) Foreign Application Priority Data
Jul. 16, 2021 (KR) 10-2021-0093690

(51) Int. Cl. H01L 51/00; C07D 471/06; C09K 11/06; C07D 409/12; C07D 403/10; C07D 403/14

(52) U.S. Cl. CPC H01L 51/0072; C07D 471/06; C09K 11/06; H01L 51/0067; C07D 409/12; H01L 51/0061; C07D 403/10; H01L 51/0052; C07D 403/14; H01L 2251/552; H01L 2251/5384; H01L 51/5016

(57) ABSTRACT

A light-emitting device includes a first emission layer and a second emission layer, wherein the first emission layer includes a first hole-transporting host (HTH1), a first electron-transporting host (ETH1), and a first phosphorescent dopant (G1), and the second emission layer includes a second hole-transporting host (HTH2), a second electron-transporting host (ETH2), and a second phosphorescent dopant (G2). The electron mobility of the ETH1 is faster than the electron mobility of the ETH2, the hole mobility of the HTH2 is faster than the hole mobility of the HTH1, and the HTH1 and/or HTH2 includes deuterium.

### CROSS-REFERENCE TO RELATED APPLICATION

This application claims priority to and the benefit of Korean Patent Application No. 10-2021-0093690, filed on Jul. 16, 2021, in the Korean Intellectual Property Office, the entire content of which is incorporated herein by reference.

### BACKGROUND

### 1. Field

One or more aspects of embodiments of the present disclosure relate to a light-emitting device and an electronic apparatus including the same.

### 2. Description of the Related Art

Light-emitting devices are self-emissive devices that, as compared with devices of the related art, may have wide viewing angles, high contrast ratios, short response times, and/or excellent or suitable characteristics in terms of luminance, driving voltage, and/or response speed.

In an example light-emitting device, a first electrode is arranged on a substrate, and a hole transport region, an emission layer, an electron transport region, and a second electrode are sequentially formed on the first electrode. Holes provided from the first electrode move toward the emission layer through the hole transport region, and electrons provided from the second electrode move toward the emission layer through the electron transport region. Carriers (such as holes and electrons) may recombine in the emission layer to produce light.

### SUMMARY

One or more aspects of embodiments of the present disclosure are directed toward a device having improved efficiency and/or a long lifespan as compared with devices of the related art.

Additional aspects will be set forth in part in the description that follows and, in part, will be apparent from the description, or may be learned by practice of the presented embodiments of the disclosure.

One or more embodiments of the present disclosure provide a light-emitting device including:

a first electrode,

a second electrode facing the first electrode, and

an interlayer between the first electrode and the second electrode, wherein:

the interlayer includes a first emission layer and a second emission layer,

the first emission layer includes a first hole-transporting host (HTH1), a first electron-transporting host (ETH1), and a first phosphorescent dopant (G1),

the second emission layer includes a second hole-transporting host (HTH2), a second electron-transporting host (ETH2), and a second phosphorescent dopant (G2),

the electron mobility of the ETH1 is faster than the electron mobility of the ETH2,

the hole mobility of the HTH2 is faster than the hole mobility of the HTH1, and

the HTH1 and/or the HTH2 includes deuterium.

One or more embodiments of the present disclosure provide an electronic apparatus including:

the light-emitting device.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic view of a light-emitting device according to an embodiment;

FIG. 2 is a cross-sectional view of a light-emitting apparatus according to an embodiment; and

FIG. 3 shows a cross-sectional view of a light-emitting apparatus according to another embodiment.

### DETAILED DESCRIPTION

Reference will now be made in more detail to embodiments, examples of which are illustrated in the accompanying drawings, wherein like reference numerals refer to like elements throughout the specification, and duplicative descriptions thereof may not be provided. In this regard, the present embodiments may have different forms and should not be construed as being limited to the descriptions set forth herein. Accordingly, the embodiments are merely described below, by referring to the drawings, to explain aspects of the present description. As used herein, the term “and/or” includes any and all combinations of one or more of the associated listed items. As used herein, expressions such as “at least one of,” “one of,” and “selected from,” when preceding a list of elements, modify the entire list of elements and do not modify the individual elements of the list. For example, the expressions “at least one of a, b and c,” “at least one of a, b or c,” and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

As used herein, singular forms such as “a,” “an,” and “the” are intended to include the plural forms as well, unless the context clearly indicates otherwise. As used herein, the terms “use,” “using,” and “used” may be considered synonymous with the terms “utilize,” “utilizing,” and “utilized,” respectively. As used herein, the term “and/or” includes any and all combinations of one or more of the associated listed items.

It will be further understood that the terms “includes,” “including,” “comprises,” and/or “comprising,” when used in this specification, specify the presence of stated features, steps, operations, elements, and/or components, but do not preclude the presence or addition of one or more other features, steps, operations, elements, components, and/or groups thereof.

The term “may” will be understood to refer to “one or more embodiments,” some of which include the described element and some of which exclude that element and/or include an alternate element. Similarly, alternative language such as “or” refers to “one or more embodiments,” each including a corresponding listed item.

It will be understood that when an element is referred to as being “on,” “connected to,” or “coupled to” another element, it may be directly on, connected, or coupled to the other element or one or more intervening elements may also be present. When an element is referred to as being “directly on,” “directly connected to,” or “directly coupled to” another element, there are no intervening elements present.

An emission layer for emitting green phosphorescence in the related art may include (e.g., consist of) a single host and a single dopant. Because the electron transport characteristics of the existing host significantly change according to the voltage, a roll-off phenomenon occurs according to the voltage change. For example, when the voltage is relatively low, luminescence occurs with normal efficiency in an emission layer, but when the voltage is relatively high, luminescence efficiency decreases in an emission layer. The reason for such a decrease of the luminescence efficiency is that surplus charges that fail to participate in luminescence are quenched (e.g., lost) via, for example, exciton-polaron or polaron-polaron quenching.

An aspect of the present disclosure provides a light-emitting device including:

a first electrode;

a second electrode facing the first electrode; and

an interlayer between the first electrode and the second electrode, wherein

the interlayer includes a first emission layer and a second emission layer,

the first emission layer includes a first hole-transporting host (HTH1), a first electron-transporting host (ETH1), and a first phosphorescent dopant (G1),

the second emission layer includes a second hole-transporting host (HTH2), a second electron-transporting host (ETH2), and a second phosphorescent dopant (G2),

electron mobility of the ETH1 is faster than that of the ETH2,

hole mobility of the HTH2 is faster than that of the HTH1, and

the HTH1 and/or the HTH2 further includes deuterium.

In an embodiment, in the light-emitting device,

the first electrode may be an anode, the second electrode may be a cathode, and the light-emitting device may further include:

a hole transport region arranged between the first electrode and the emission layer and including a hole injection layer, a hole transport layer, an electron blocking layer, or any combination thereof; and/or

an electron transport region arranged between the second electrode and the emission layer and including a hole blocking layer, an electron transport layer, an electron injection layer, or any combination thereof.

For example, the first emission layer and the electron blocking layer may be in contact with each other. For example, the first emission layer may be directly on the electron blocking layer.

For example, the second emission layer and the hole blocking layer may be in contact with each other. For example, the second emission layer may be directly on the hole blocking layer.

Because the first emission layer includes the HTH1 having slower (e.g., lower) hole mobility than the HTH2, the hole control function is strengthened, and because the second emission layer includes the ETH2 having slower (e.g., lower) electron mobility than the ETH1, the electron control function is strengthened.

For example, the electron mobility of the ETH1 may be 1.0×10¹ to 1.0×10³ (e.g., 10 to 1000) times faster than the electron mobility of the ETH2.

For example, the electron mobility of the ETH1 may be in a range of about 1.0×10⁻⁵V·s/cm² to about 1.0×10⁻²V·s/cm², and the electron mobility of the ETH2 may be in a range of about 1.0×10⁻⁷V·s/cm² to about 5.0×10⁻³V·s/cm²

The HTH1 and the HTH2 may each independently have a hole mobility in a range of, for example, about 1.0×10⁻⁶ V·s/cm² to about 1.0×10⁻⁴ V·s/cm².

In an embodiment, only the HTH1 and/or the HTH2 may include deuterium, and neither of the ETH1 or the ETH2 may include deuterium.

In an embodiment, the HTH1 and/or the HTH2 may further include hydrogen, and a ratio of hydrogen to deuterium in each of the HTH1 and/or the HTH2 may be in a range of about 1:9 to about 9:1.

For example, the HTH1 and/or the HTH2 may each independently have a ratio of hydrogen to deuterium in a range of about 2:8 to about 8:2. For example, the HTH1 and/or the HTH2 may each independently have a ratio of hydrogen to deuterium in a range of about 3:7 to about 7:3. For example, the HTH1 and/or the HTH2 may each independently have a ratio of hydrogen to deuterium in a range of about 4:6 to about 6:4. For example, the HTH1 and/or the HTH2 may each independently have a ratio of hydrogen to deuterium in a range of about 5:5 to about 9:1.

When the number (e.g., concentration or proportion) of deuterium is too small, desired or suitable molecular rigidity may not be obtained, and substituting most hydrogen atoms in a compound with deuterium (e.g., deuterating the compound and/or region) may be expensive.

In an embodiment, the first emission layer and the second emission layer may be in contact with each other. For example, the first emission layer and the second emission layer may physically be in direct contact with each other.

In an embodiment, the HTH1 and the HTH2 may be different from each other, and the ETH1 and the ETH2 may be different from each other. For example, the HTH1, the HTH2, the ETH1, and the ETH2 may be different from each other (e.g., may have different compositions and/or properties).

In an embodiment, an absolute value of a lowest unoccupied molecular orbital (LUMO) energy level of the ETH1 may be greater than that of a LUMO energy level of the ETH2. In one or more embodiments, the LUMO energy level of the ETH1 may be deeper (e.g., more negative) than that of the ETH2.

For example, the LUMO energy of the ETH1 and the LUMO energy of the ETH2 may satisfy Condition (1):

0.01 eV≤|E_{LUMO_ETH2}−E_{LUMO_ETH1}|≤0.50 eV (1)

In an embodiment, an absolute value of a highest occupied molecular orbital (HOMO) energy level of the HTH1 may be greater than that of a HOMO energy level of the HTH2. In one or more embodiments, the HOMO energy level of the HTH1 may be deeper (e.g., more negative) than that of the HTH2.

For example, the HOMO energy of the HTH1 and the HOMO energy of the HTH2 may satisfy Condition (2):

0.01 eV≤|E_{HOMO_HTH2}−E_{HOMO_HTH1}|≤0.50 eV (2)

When the energy relationships between the molecular orbital functions of the HTH1, the ETH1, the HTH2, and the ETH2, the electron mobility of the ETH and the ETH1, and the hole mobility of the HTH1 and the HTH2 are the same as respectively described above, exciton-polaron quenching and polaron-polaron quenching, which occur in the case of high field (e.g., at high voltages), may be suppressed or reduced, so that the roll-off phenomenon may be also reduced and accordingly, the light-emitting device may have improved efficiency characteristics.

Furthermore, when the HTH1 and/or the HTH2 includes deuterium, the light-emitting device may have improved lifespan characteristics.

The hole-transporting host may be a compound having strong hole properties. The expression “a compound having strong hole properties” refers to a compound that can easily accept holes, and such properties may be obtained by including a hole-receiving moiety (also referred to as a HT moiety).

Such a HT moiety may include, for example, a π-electron-rich heteroaromatic compound (e.g., a carbazole derivative or an indole derivative), or an aromatic amine compound.

The electron-transporting host may be a compound having strong electron properties. The expression “a compound having strong electron properties” refers to a compound that can easily accept electrons, and such properties may be obtained by including an electron-receiving moiety (also referred to as an ET moiety).

Such an ET moiety may include, for example, a π electron-deficient heteroaromatic compound. For example, the ET moiety may include a nitrogen-containing heteroaromatic compound.

When a compound includes only a HT moiety or only an ET moiety, it is clear (e.g., it may be predictable) whether the compound has HT properties or ET properties (e.g., predominantly).

In an embodiment, a compound may include both a HT moiety and an ET moiety (e.g., simultaneously). In this regard, a simple comparison between the total number of the HT moieties and the total number of the ET moieties in the compound can be a criterion for predicting whether the compound may function as a HT compound or an ET compound, but cannot be an absolute criterion. One of the reasons why such a simple comparison cannot provide an absolute criterion is that one HT moiety and one ET moiety may not have exactly the same (e.g., equal) ability to attract holes and electrons.

Therefore, in order to determine whether a compound of a given structure is a HT compound or an ET compound, a simulation may be made in advance for prediction, and finally, direct implementation may be made in a device including the compound to relatively reliably confirm the properties of the compound.

In an embodiment, the HTH1 and the HTH2 may each independently be represented by one of Formulae 311-1 to 311-6, and the ETH1 and the ETH1 may each independently be represented by one of Formulae 312-1 to 312-4 and 313:

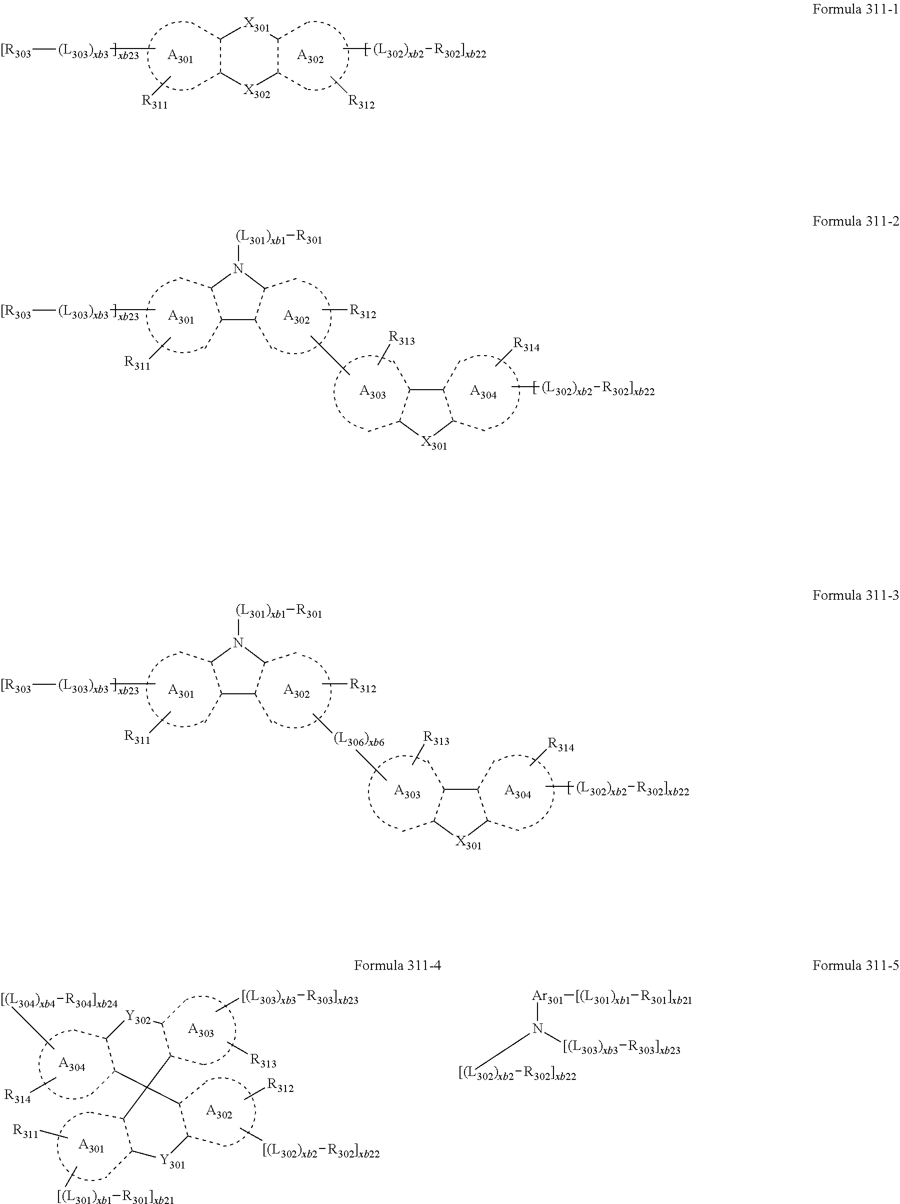

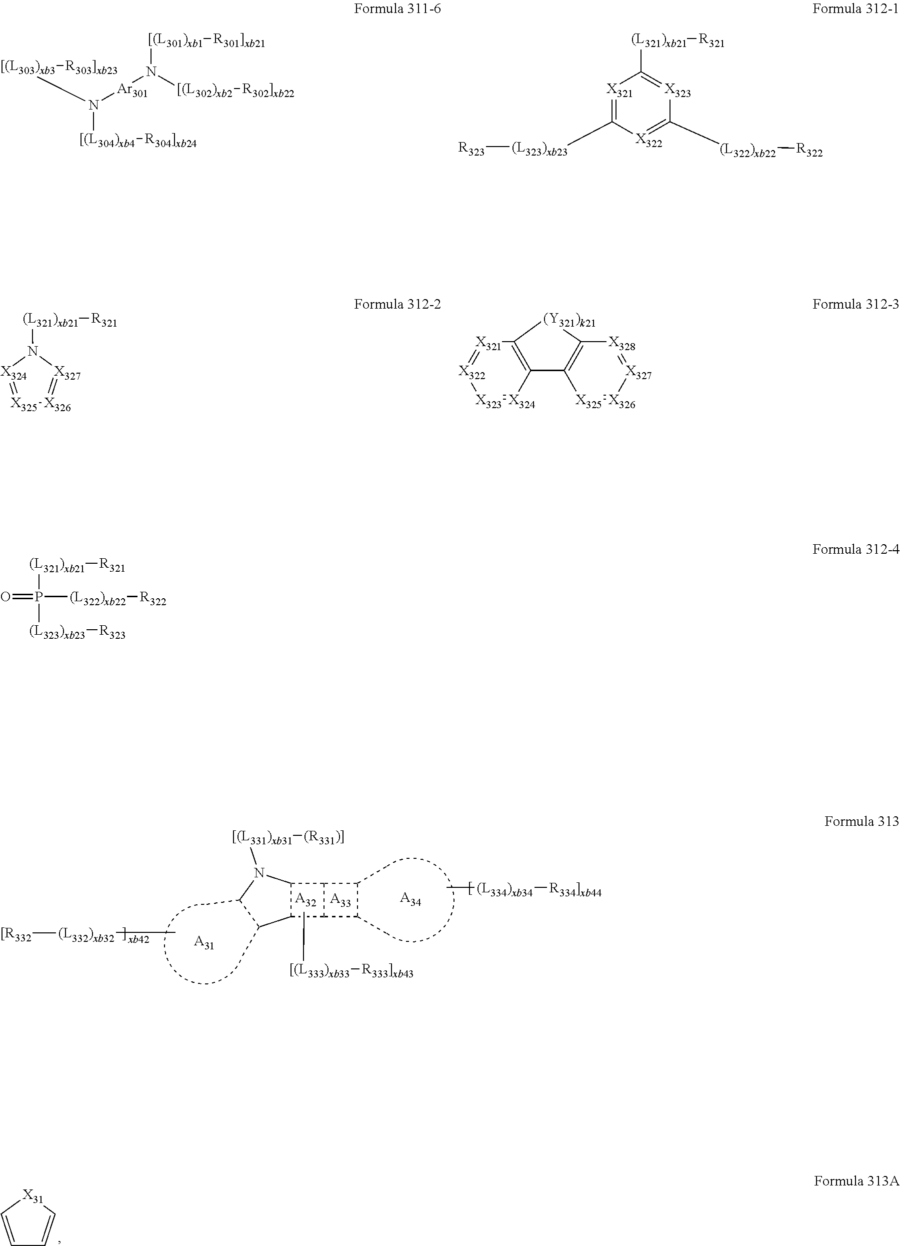

wherein, in Formulae 311-1 to 311-6, 312-1 to 312-4, 313, and 313 Å,

Ar₃₀₁ may be a C₃-C₆₀ carbocyclic group unsubstituted or substituted with at least one R₁₀ₐ or a C₁-C₆₀ heterocyclic group unsubstituted or substituted with at least one R₁₀ₐ,

Ar₃₀₁ to Ar₃₀₄ may each independently be a C₃-C₆₀ carbocyclic group or a C₁-C₆₀ heterocyclic group,

X₃₀₁ may be O, S, N-[(L₃₀₄)_{xb4}-R₃₀₄], C[(L₃₀₄)_{xb4}-R₃₀₄][(L₃₀₅)_{xb5}-R₃₀₅], or Si[(L₃₀₄)_{xb4}-R₃₀₄][(L₃₀₅)_{xb5}-R₃₀₅],

X₃₀₂, Y₃₀₁, and Y₃₀₂ may each independently be a single bond, O, S, N-[(L₃₀₅)_{xb5}-R₃₀₅], C[(L₃₀₄)_{xb4}-R₃₀₄][(L₃₀₅)_{xb5}-R₃₀₅], Si[(L₃₀₄)_{xb4}-R₃₀₄][(L₃₀₅)_{xb5}-R₃₀₅], or S(═O)₂,

xb1 to xb5 may each independently be 0, 1, 2, 3, 4, or 5,

xb6 may be 1, 2, 3, 4, or 5,

X₃₂₁ to X₃₂₈ may each independently be N or C[(L₃₂₄)_{xb24}-R₃₂₄],

Y₃₂₁ may be *—O—*′, *—S—*′, *—N[(L₃₂₅)_{xb25}-R₃₂₅]—*′, *—C[(L₃₂₅)_{xb25}-R₃₂₅][(L₃₂₆)_{xb26}-R₃₂₆]—*′, *—C[(L₃₂₅)_{xb25}-R₃₂₅]═C[(L₃₂₆)_{xb26}-R₃₂₆]—*′, *—C[(L₃₂₅)_{xb25}-R₃₂₅]═N—*′, or *—N═C[(L₃₂₆)_{xb26}-R₃₂₆]—*′,

k21 may be 0, 1, or 2, wherein Y₃₂₁ does not exist when k21 is 0,

xb21 to xb26 may each independently be 0, 1, 2, 3, 4, or 5,

A₃₁, A₃₂, and A₃₄ may each independently be a C₃-C₆₀ carbocyclic group or a C₁-C₃₀ heterocyclic group,

A₃₃ may be a group represented by Formula 313 Å,

X₃₁ may be N[(L₃₃₅)_{xb35}-(R₃₃₅)], O, S, Se, C[L₃₃₅)_{xb35}- (R₃₃₅)][(L₃₃₆)_{xb36}-(R₃₃₆)], or Si[L₃₃₅)_{xb35}-(R₃₃₅)][(L₃₃₆)_{xb36}-(R₃₃₆)],

xb31 to xb36 may each independently be 0, 1, 2, 3, 4, or 5,

xb42 to xb44 may each independently be 0, 1, 2, 3, 4, 5, 6, 7, 8, 9, or 10,

L₃₀₁ to L₃₀₆, L₃₂₁ to L₃₂₆, and L₃₃₁ to L₃₃₆ may each independently be a single bond, a C₁-C₂₀ alkylene group that is unsubstituted or substituted with at least one R₁₀ₐ, a C₁-C₂₀ alkenylene group that is unsubstituted or substituted with at least one R₁₀ₐ, a C₁-C₂₀ alkynylene group that is unsubstituted or substituted with at least one R₁₀ₐ, a C₃-C₁₀ cycloalkylene group that is unsubstituted or substituted with at least one R₁₀ₐ, a C₁-C₁₀ heterocycloalkylene group that is unsubstituted or substituted with at least one R₁₀ₐ, a C₃-C₁₀ cycloalkenylene group that is unsubstituted or substituted with at least one R₁₀ₐ, a C₁-C₁₀ heterocycloalkenylene group that is unsubstituted or substituted with at least one R₁₀ₐ, a C₆-C₆₀ arylene group that is unsubstituted or substituted with at least one R₁₀ₐ, a C₁-C₆₀ heteroarylene group that is unsubstituted or substituted with at least one R₁₀ₐ, a divalent non-aromatic condensed polycyclic group that is unsubstituted or substituted with at least one R₁₀ₐ, or a divalent non-aromatic condensed heteropolycyclic group that is unsubstituted or substituted with at least one R₁₀ₐ,

R₃₀₁ to R₃₀₅, R₃₁₁ to R₃₁₄, R₃₂₁ to R₃₂₆, and R₃₃₁ to R₃₃₆ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),

two or more neighboring substituents of R₃₀₃, R₃₀₄, R₃₁₁, and R₃₂₁ to R₃₂₄ may optionally be bonded to each other to form a C₃-C₆₀ carbocyclic group unsubstituted or substituted with at least one R₁₀ₐ or a C₁-C₆₀ heterocyclic group unsubstituted or substituted with at least one R₁₀ₐ,

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and

Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

Formula 311-1 may include, for example, an indolocarbazole moiety, or may be represented by Formula 311-1′:

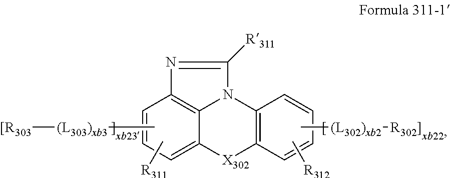

wherein, in Formula 311-1′, R′₃₁₁ may be the same as described in connection with R₃₁₁, xb23′ may be the same as described in connection with xb23, and the remaining substituents and subscripts may be the same as described in connection with Formula 311-1.

In an embodiment, the HTH1 and the HTH2 may each independently be selected from Compounds 1-1 to 1-14:

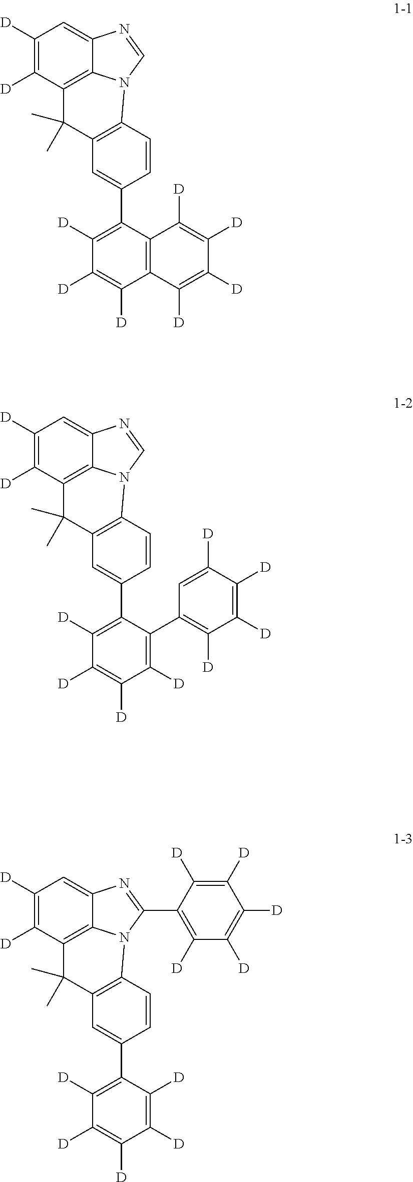 [SMILES: [2H]c1cc2ncn3c2c(c1[2H])C(C)(C)c1cc(-c2c([2H])c([2H])c([2H])c4c([2H])c([2H])c([2H])c([2H])c24)ccc1-3 ; [2H]c1cc(-c2c([2H])c([2H])c([2H])c([2H])c2-c2ccc3c(c2)C(C)(C)c2c([2H])c([2H])cc4ncn-3c24)c([2H])c([2H])c1[2H] ; [2H]c1cc2nc(-c3c([2H])c([2H])c([2H])c([2H])c3[2H])n3c2c(c1[2H])C(C)(C)c1cc(-c2c([2H])c([2H])c([2H])c([2H])c2[2H])ccc1-3]

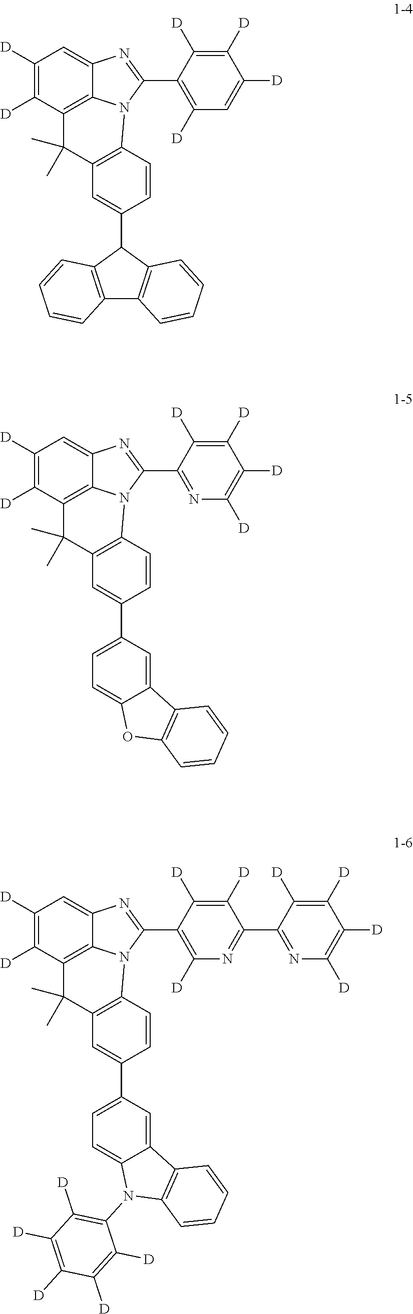 [SMILES: [2H]c1cc([2H])c(-c2nc3cc([2H])c([2H])c4c3n2-c2ccc(C3c5ccccc5-c5ccccc53)cc2C4(C)C)c([2H])c1[2H] ; [2H]c1cc2nc(-c3nc([2H])c([2H])c([2H])c3[2H])n3c2c(c1[2H])C(C)(C)c1cc(-c2ccc4oc5ccccc5c4c2)ccc1-3 ; [2H]c1cc2nc(-c3c([2H])nc(-c4nc([2H])c([2H])c([2H])c4[2H])c([2H])c3[2H])n3c2c(c1[2H])C(C)(C)c1cc(-c2ccc4c(c2)c2ccccc2n4-c2c([2H])c([2H])c([2H])c([2H])c2[2H])ccc1-3]

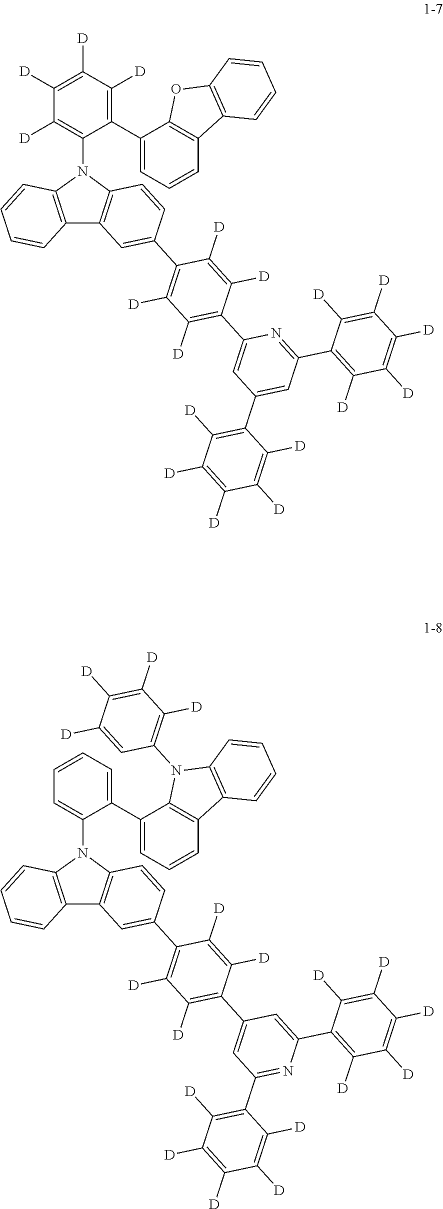 [SMILES: [2H]c1c([2H])c([2H])c(-c2cc(-c3c([2H])c([2H])c([2H])c([2H])c3[2H])nc(-c3c([2H])c([2H])c(-c4ccc5c(c4)c4ccccc4n5-c4c([2H])c([2H])c([2H])c([2H])c4-c4cccc5c4oc4ccccc45)c([2H])c3[2H])c2)c([2H])c1[2H] ; [2H]c1cc(-n2c3ccccc3c3cccc(-c4ccccc4-n4c5ccccc5c5cc(-c6c([2H])c([2H])c(-c7cc(-c8c([2H])c([2H])c([2H])c([2H])c8[2H])nc(-c8c([2H])c([2H])c([2H])c([2H])c8[2H])c7)c([2H])c6[2H])ccc54)c32)c([2H])c([2H])c1[2H]]

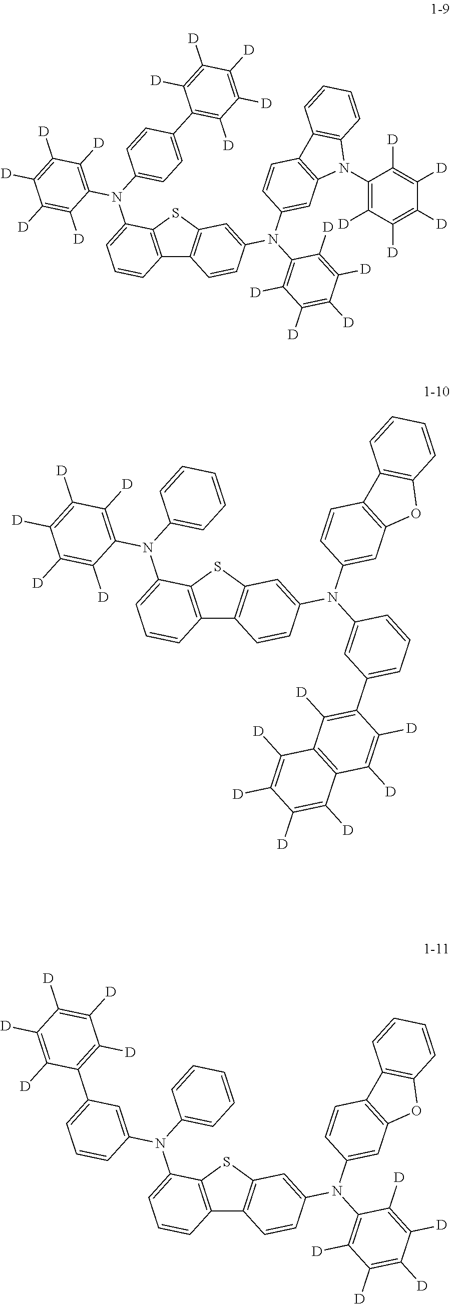

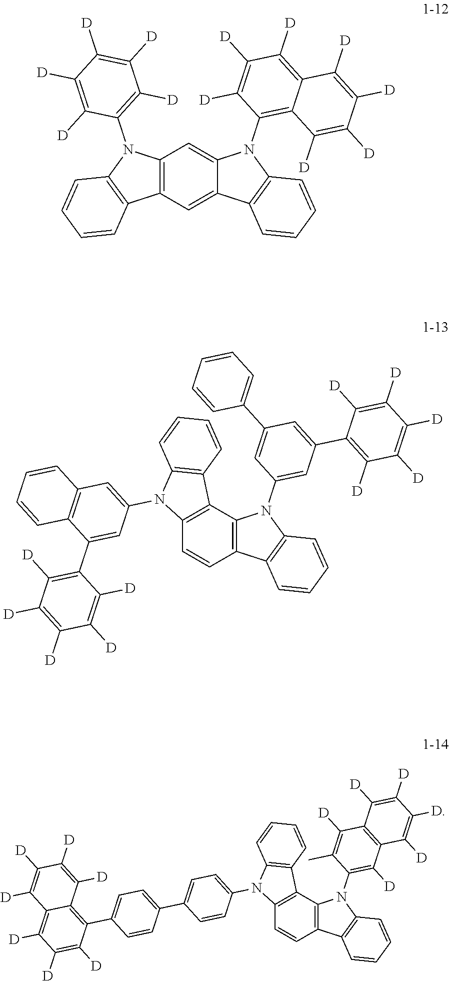

In an embodiment, the ETH1 and the ETH2 may each independently be selected from Compounds 2-1 to 2-20:

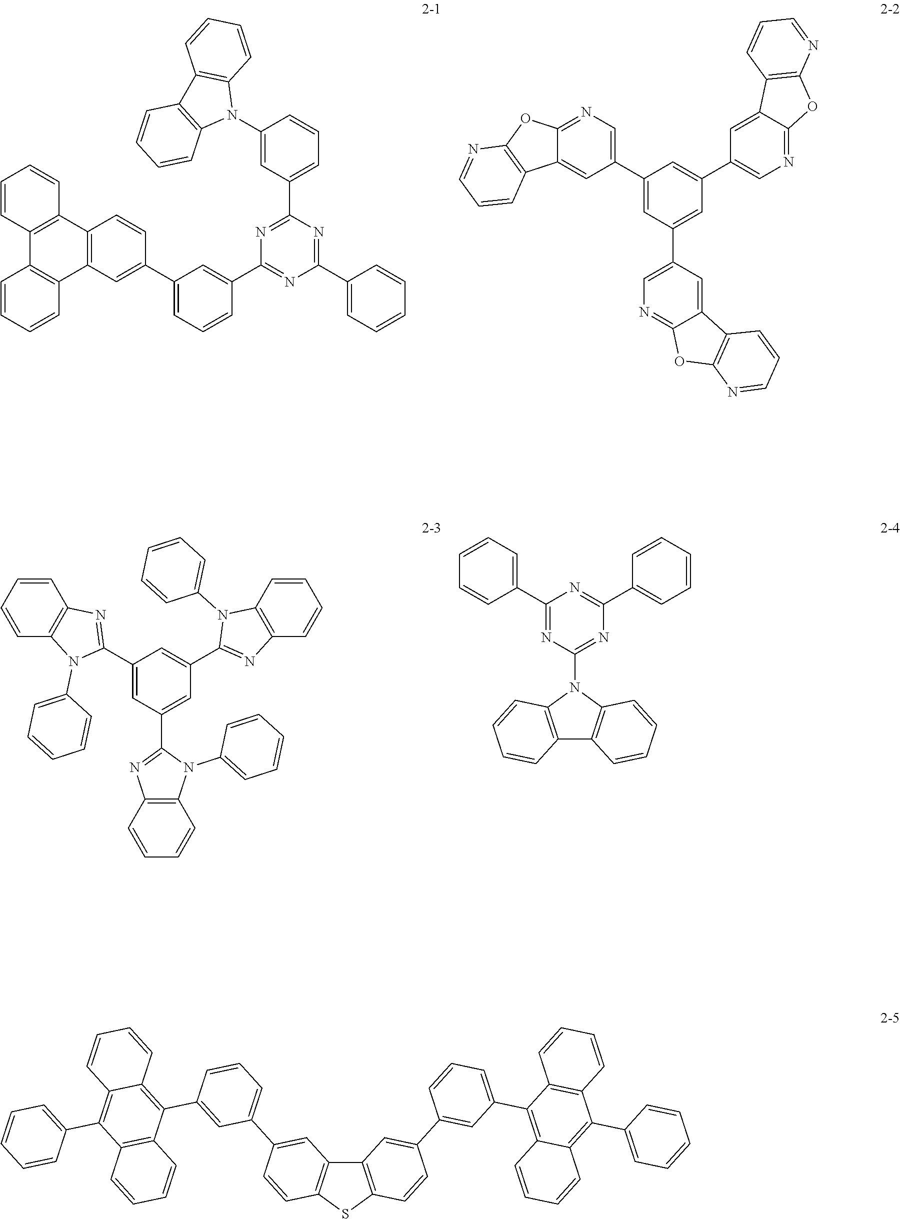 [SMILES: c1ccc(-c2nc(-c3cccc(-c4ccc5c6ccccc6c6ccccc6c5c4)c3)nc(-c3cccc(-n4c5ccccc5c5ccccc54)c3)n2)cc1 ; c1cnc2oc3ncc(-c4cc(-c5cnc6oc7ncccc7c6c5)cc(-c5cnc6oc7ncccc7c6c5)c4)cc3c2c1 ; c1ccc(-n2c(-c3cc(-c4nc5ccccc5n4-c4ccccc4)cc(-c4nc5ccccc5n4-c4ccccc4)c3)nc3ccccc32)cc1 ; c1ccc(-c2nc(-c3ccccc3)nc(-n3c4ccccc4c4ccccc43)n2)cc1 ; c1ccc(-c2c3ccccc3c(-c3cccc(-c4ccc5sc6ccc(-c7cccc(-c8c9ccccc9c(-c9ccccc9)c9ccccc89)c7)cc6c5c4)c3)c3ccccc23)cc1]

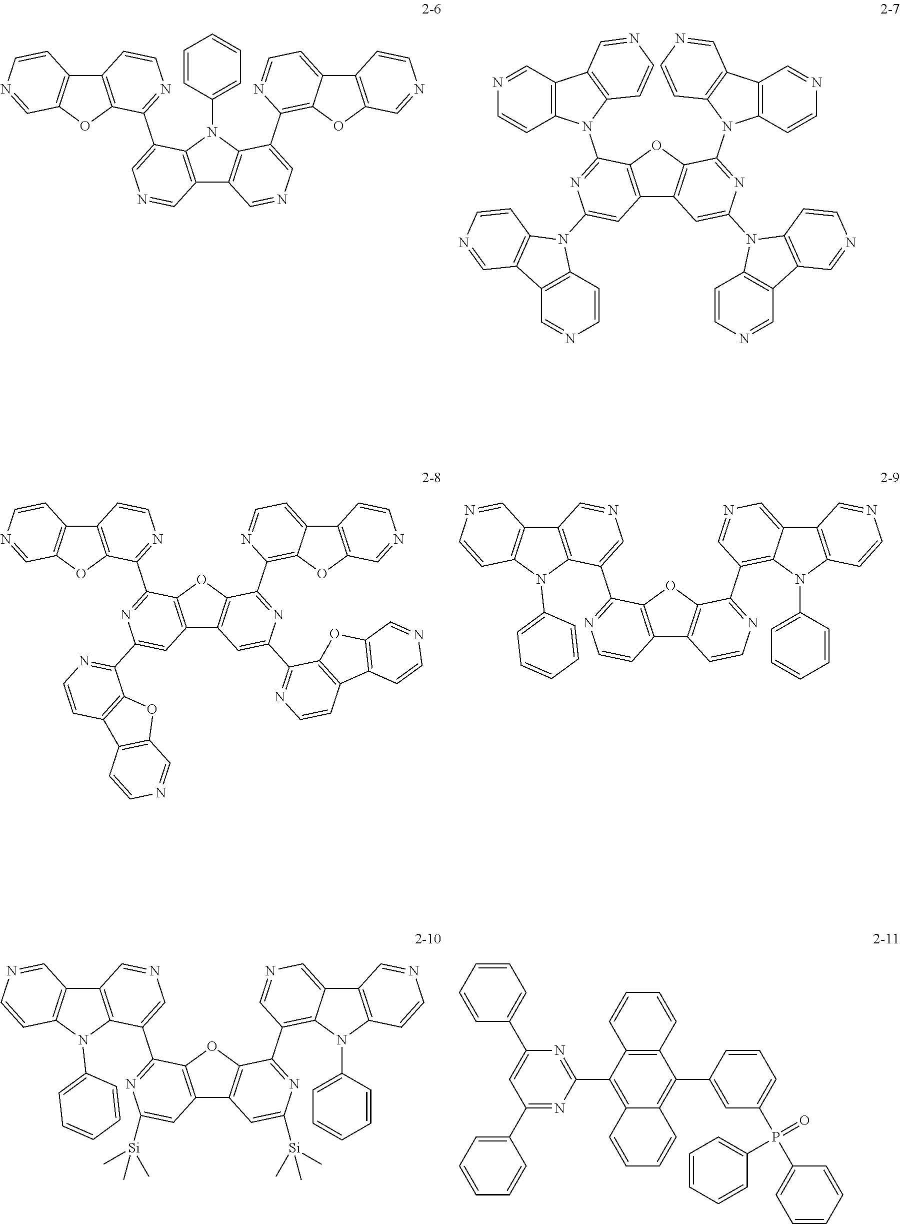

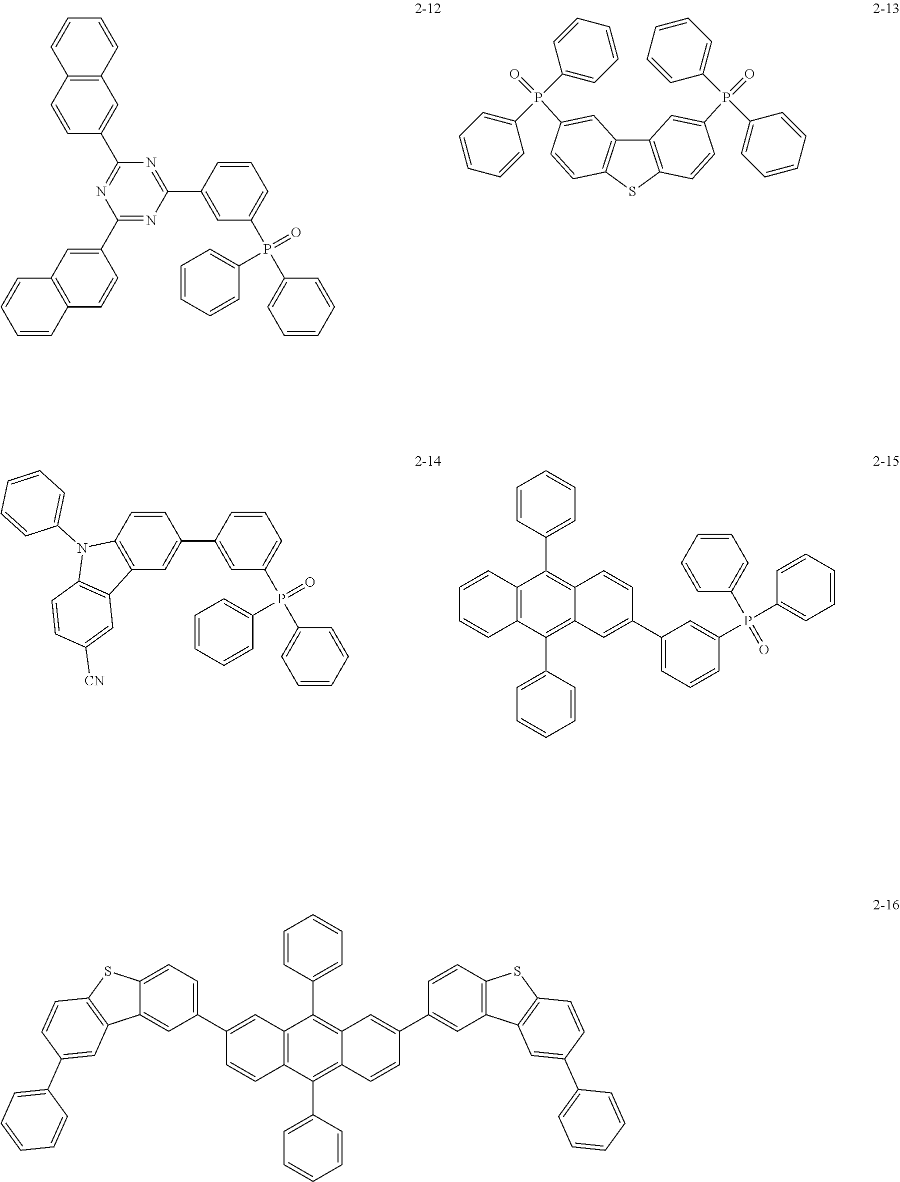 [SMILES: O=P(c1ccccc1)(c1ccccc1)c1cccc(-c2nc(-c3ccc4ccccc4c3)nc(-c3ccc4ccccc4c3)n2)c1 ; O=P(c1ccccc1)(c1ccccc1)c1ccc2sc3ccc(P(=O)(c4ccccc4)c4ccccc4)cc3c2c1 ; N#Cc1ccc2c(c1)c1cc(-c3cccc(P(=O)(c4ccccc4)c4ccccc4)c3)ccc1n2-c1ccccc1 ; O=P(c1ccccc1)(c1ccccc1)c1cccc(-c2ccc3c(-c4ccccc4)c4ccccc4c(-c4ccccc4)c3c2)c1 ; c1ccc(-c2ccc3sc4ccc(-c5ccc6c(-c7ccccc7)c7ccc(-c8ccc9sc%10ccc(-c%11ccccc%11)cc%10c9c8)cc7c(-c7ccccc7)c6c5)cc4c3c2)cc1]

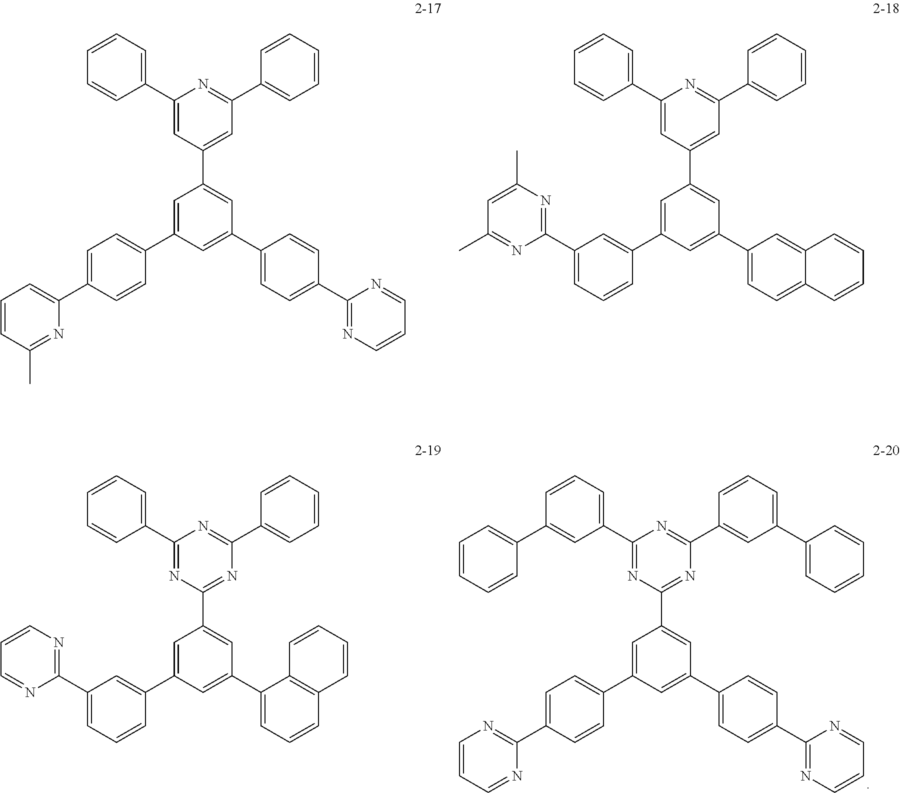 [SMILES: Cc1cccc(-c2ccc(-c3cc(-c4ccc(-c5ncccn5)cc4)cc(-c4cc(-c5ccccc5)nc(-c5ccccc5)c4)c3)cc2)n1 ; Cc1cc(C)nc(-c2cccc(-c3cc(-c4cc(-c5ccccc5)nc(-c5ccccc5)c4)cc(-c4ccc5ccccc5c4)c3)c2)n1 ; c1ccc(-c2nc(-c3ccccc3)nc(-c3cc(-c4cccc(-c5ncccn5)c4)cc(-c4cccc5ccccc45)c3)n2)cc1 ; c1ccc(-c2cccc(-c3nc(-c4cccc(-c5ccccc5)c4)nc(-c4cc(-c5ccc(-c6ncccn6)cc5)cc(-c5ccc(-c6ncccn6)cc5)c4)n3)c2)cc1]

In an embodiment, the G1 and the G2 may be identical to or different from each other.

In an embodiment, the G1 and the G2 may each independently have a maximum emission wavelength in a range of about 490 nm to about 560 nm.

In one or more embodiments, the G1 and the G2 may each independently have a maximum emission wavelength in a range of about 520 nm to about 540 nm.

In an embodiment, a difference between a maximum emission wavelength of the G1 and a maximum emission wavelength of the G2 may be about 10 nm or less. In one or more embodiments, the difference between the maximum emission wavelength of the G1 and the maximum emission wavelength of the G2 may be about 5 nm or less or about 3 nm or less.

In an embodiment, a coincidence ratio between an emission spectrum of the G1 and an emission spectrum of the G2 may be about 80% or more. In one or more embodiments, the coincidence ratio between the emission spectrum of the G1 and the emission spectrum of the G2 may be about 85% or more, about 90% or more, and about 100% or less.

For example, an area of a region where the emission spectrum of the G1 and the emission spectrum of the G2 overlap may be, based on the emission spectrum region of the G1, about 85% or more, about 90% or more, and about 100% or less.

In an embodiment, the G1 and the G2 may each independently be a compound represented by Formula 411 or 412:

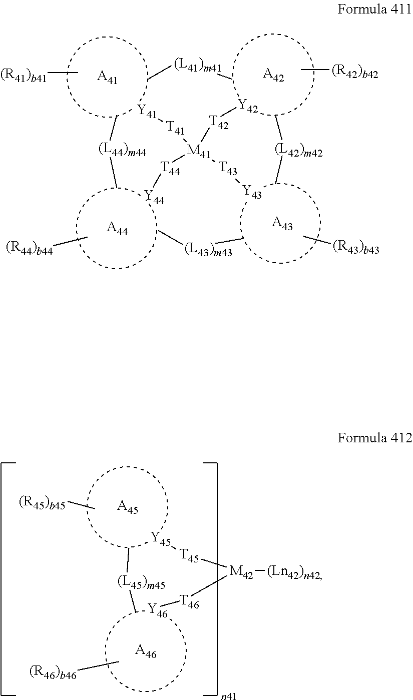

wherein, in Formulae 411 and 412,

M₄₁ may be platinum (Pt), palladium (Pd), copper (Cu), silver (Ag), gold (Au), rhodium (Rh), iridium (Ir), ruthenium (Ru), osmium (Os), titanium (Ti), zirconium (Zr), hafnium (Hf), europium (Eu), terbium (Tb), or thulium (Tm),

n41 may be 1, 2, or 3,

Ln₄₂ may be an organic ligand, and n42 may be 0, 1, or 2,

Y₄₁ to Y₄₆ may each independently be N or C,

A₄₁ to A₄₆ may each independently be a C₃-C₆₀ carbocyclic group or a C₁-C₆₀ heterocyclic group,

T₄₁ to T₄₆ may each independently be a single bond, *—O—*′, or *—S—*′,

L₄₁ to L₄₅ may each independently be a single bond, *—O—*′, *—S—*′, *—C(R₄₇)(R₄₈)—*′, *—C(R₄₇)=*′, *═C(R₄₇)—*′, *—C(R₄₇)═C(R₄₈)—*′, *—C(═O)—*′, *—C(═S)—*′, *—C≡C—*′, *—B(R₄₇)—*′, *—N(R₄₇)—*′, *—P(R₄₇)—*′, *—Si(R₄₇)(R₄₈)—*′, *—P(═O)(R₄₇)—*′, or *—Ge(R₄₇)(R₄₈)—*′,

m41 to m45 may each independently be 0, 1, 2, or 3,

R₄₁ to R₄₈ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₄₁)(Q₄₂)(Q₄₃), —N(Q₄₁)(Q₄₂), —B(Q₄₁)(Q₄₂), —C(═O)(Q₄₁), S(═O)₂(Q₄₁), or —P(═O)(Q₄₁)(Q₄₂),

R₄₇ and R₄₁; R₄₇ and R₄₂; R₄₇ and R₄₃; or R₄₇ and R₄₄ may optionally be linked together to form a C₃-C₆₀ carbocyclic group unsubstituted or substituted with at least one R₁₀ₐ or a C₁-C₆₀ heterocyclic group unsubstituted or substituted with at least one R₁₀ₐ,

b41 to b46 may each independently be 1, 2, 3, 4, 5, 6, 7, or 8,

* and *′ each indicate a binding site to a neighboring atom,

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and

Q₁₁ to Q₁₃, Q₂₁ to Q₂₃, Q₃₁ to Q₃₃, and Q₄₁ to Q₄₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, the G1 and the G2 may each independently be selected from Compounds G-1 to G-12:

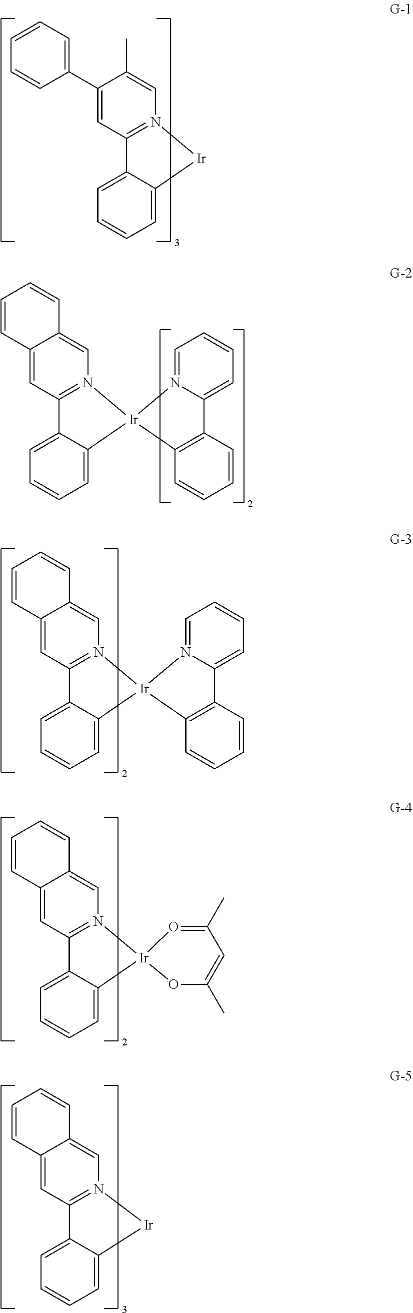

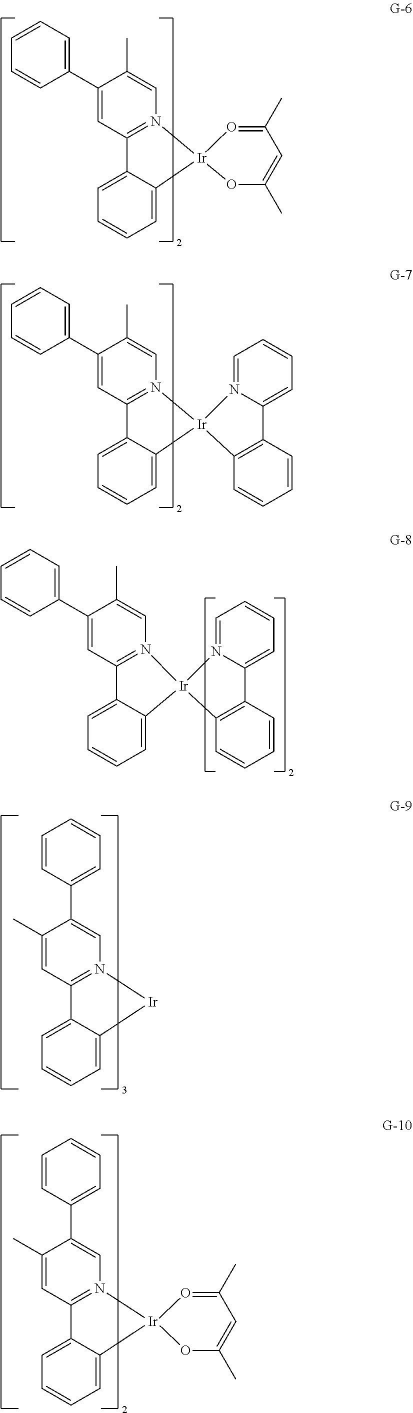

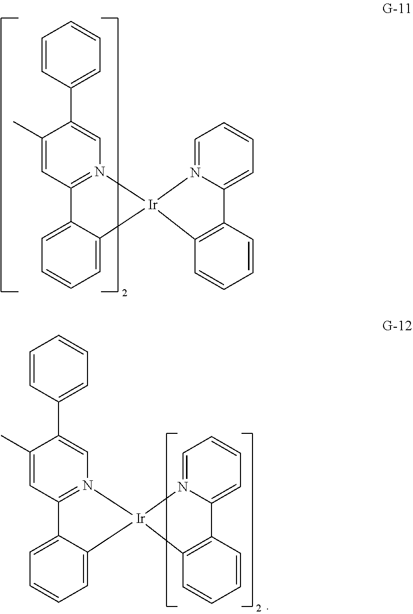

Another aspect of the present disclosure provides an electronic apparatus including the light-emitting device.

In an embodiment, the electron apparatus may further include a thin-film transistor, wherein:

the thin-film transistor may include a source electrode and a drain electrode, and

the first electrode of the light-emitting device may be electrically connected to at least one of the source electrode or the drain electrode of the thin-film transistor.

In an embodiment, the electronic apparatus may further include a color filter, a color conversion layer, a touch screen layer, a polarizing layer, or any combination thereof.

The term “interlayer” as utilized herein may refer to a single layer and/or all of a plurality of layers between the first electrode and the second electrode of the light-emitting device.

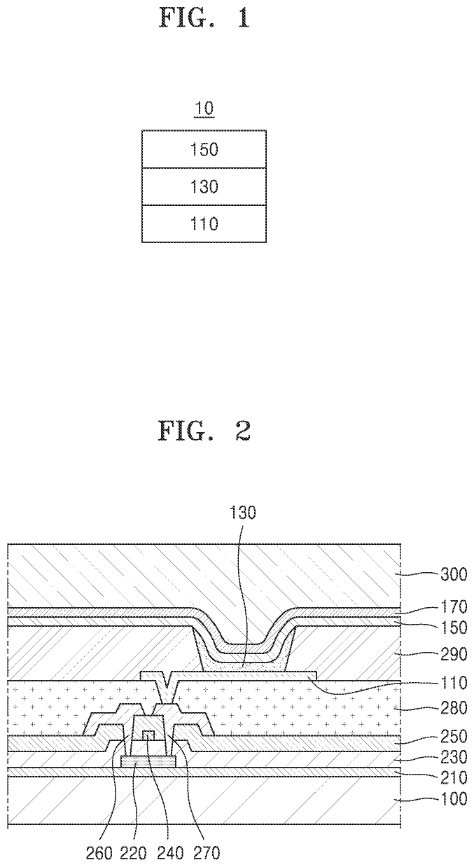

### Description of FIG. 1

FIG. 1 is a schematic cross-sectional view of a light-emitting device 10 according to an embodiment. The light-emitting device 10 includes a first electrode 110, an interlayer 130, and a second electrode 150.

Hereinafter, the structure of the light-emitting device 10 according to an embodiment and a method of manufacturing the light-emitting device 10 will be described with reference to FIG. 1.

### First Electrode 110

In FIG. 1, a substrate may be additionally arranged under the first electrode 110 or above the second electrode 150. In an embodiment, the substrate may be a glass substrate and/or a plastic substrate. In one or more embodiments, the substrate may be a flexible substrate, and for example, may include plastics with excellent or suitable heat resistance and/or durability (such as polyimide, polyethylene terephthalate (PET), polycarbonate, polyethylene naphthalate, polyarylate (PAR), polyetherimide, or any combination thereof).

The first electrode 110 may be formed by, for example, depositing or sputtering a material for forming the first electrode 110 on the substrate. When the first electrode 110 is an anode, a material for forming the first electrode 110 may be a high-work function material to facilitate injection of holes.

The first electrode 110 may be a reflective electrode, a semi-transmissive electrode, or a transmissive electrode. In an embodiment, when the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure including (e.g., consisting of) a single layer or a multi-layered structure including a plurality of layers. For example, the first electrode 110 may have a three-layered structure of ITO/Ag/ITO.

### Interlayer 130

The interlayer 130 is on the first electrode 110. The interlayer 130 may include an emission layer.

The interlayer 130 may further include a hole transport region between the first electrode 110 and the emission layer and an electron transport region between the emission layer and the second electrode 150.

In an embodiment, the interlayer 130 may further include, in addition to one or more suitable organic materials, a metal-containing compound (such as an organometallic compound), an inorganic material (such as a quantum dot), and/or the like.

In one or more embodiments, the interlayer 130 may include i) two or more emission layers sequentially stacked between the first electrode 110 and the second electrode 150 and ii) a charge generation layer located between the two or more emission layers. When the interlayer 130 includes the emission layer and the charge generation layer as described above, the light-emitting device 10 may be a tandem light-emitting device.

### Hole Transport Region in Interlayer 130

The hole transport region 120 may have i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The hole transport region 120 may include a hole injection layer, a hole transport layer, an emission auxiliary layer, an electron blocking layer, or any combination thereof.

For example, the hole transport region 120 may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron-blocking layer structure, wherein the layers of each structure are sequentially stacked from the first electrode 110.

For example, the hole transport region may have a multi-layered structure of hole transport layer/emission auxiliary layer, or hole transport layer/electron blocking layer, which are sequentially stacked in this stated order from the first electrode 110.

The hole transport region may include a compound represented by Formula 201, a compound represented by Formula 202, or any combination thereof:

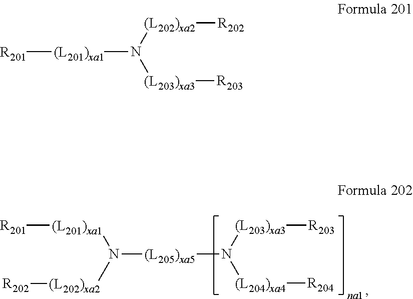

wherein, in Formulae 201 and 202,

L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently be an integer from 0 to 5,

xa5 may be an integer from 1 to 10,

R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (for example, a carbazole group and/or the like) unsubstituted or substituted with at least one R₁₀ₐ (for example, Compound HT16),

R₂₀₃ and R₂₀₄ may optionally be linked to each other, via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and

na1 may be an integer from 1 to 4.

For example, each of Formulae 201 and 202 may include at least one of the groups represented by Formulae CY201 to CY217:

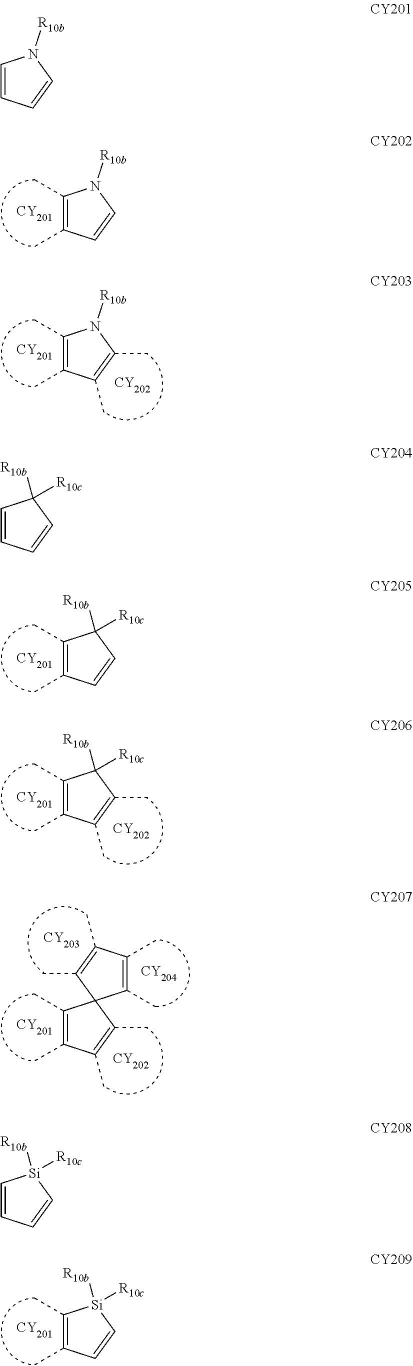

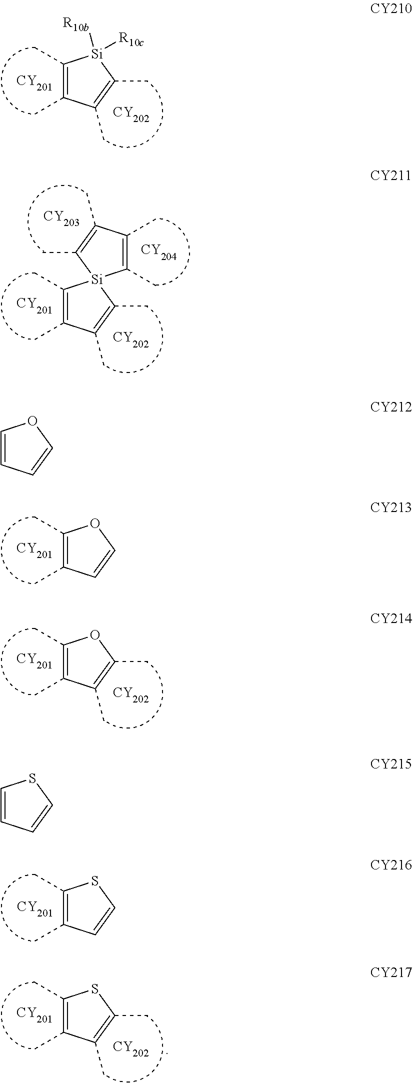

R_{10b} and R_{10c} in Formulae CY201 to CY217 may each independently be the same as described in connection with R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one of the groups represented by Formulae CY201 to CY203.

In one or more embodiments, Formula 201 may include at least one of the groups represented by Formulae CY201 to CY203 and at least one of the groups represented by Formulae CY204 to CY217.

In one or more embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In one or more embodiments, each of Formulae 201 and 202 may not include (e.g., may exclude) a group represented by one of Formulae CY201 to CY203.

In one or more embodiments, each of Formulae 201 and 202 may not include (e.g., may exclude) a group represented by one of Formulae CY201 to CY203, and may include at least one of groups represented by Formulae CY204 to CY217.

In one or more embodiments, each of Formulae 201 and 202 may not include (e.g., may exclude) a group represented by one of Formulae CY201 to CY217.

For example, the hole transport region may include one of Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzene sulfonic acid (PANI/DBSA), poly(3,4-ethylene dioxythiophene)/poly(4-styrene sulfonate) (PEDOT/PSS), polyaniline/camphor sulfonic acid (PANI/CSA), polyaniline/poly(4-styrene sulfonate) (PANI/PSS), or any combination thereof:

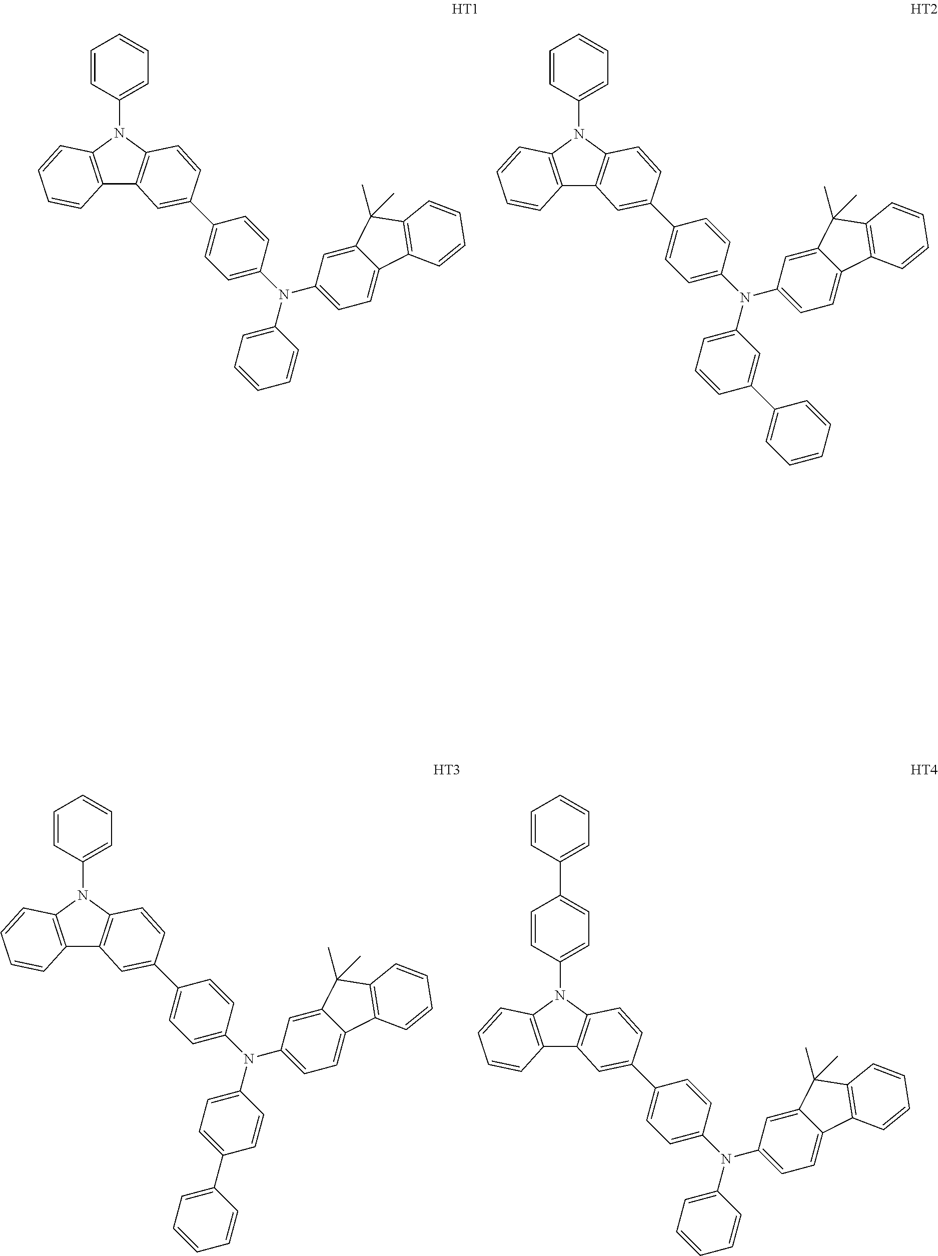 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

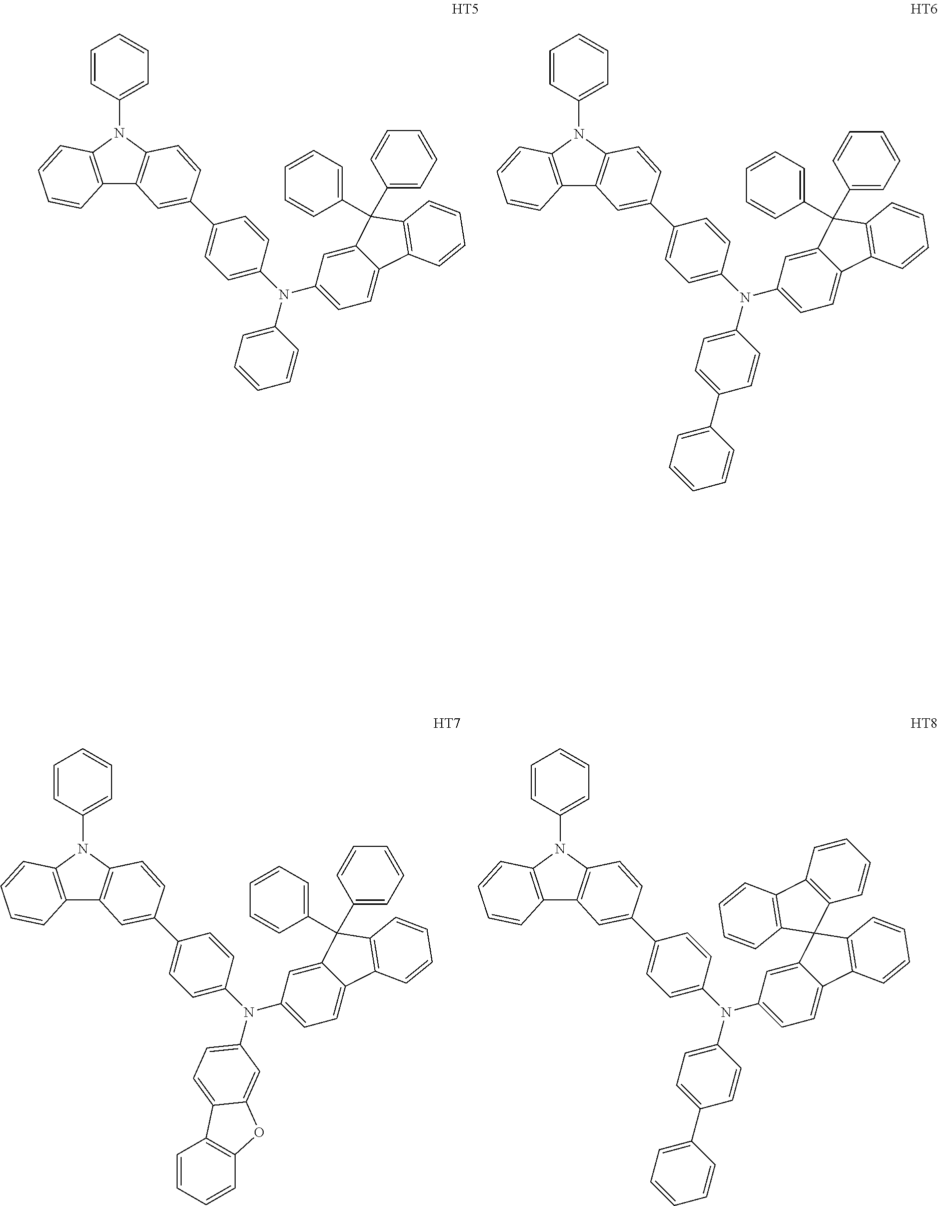 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

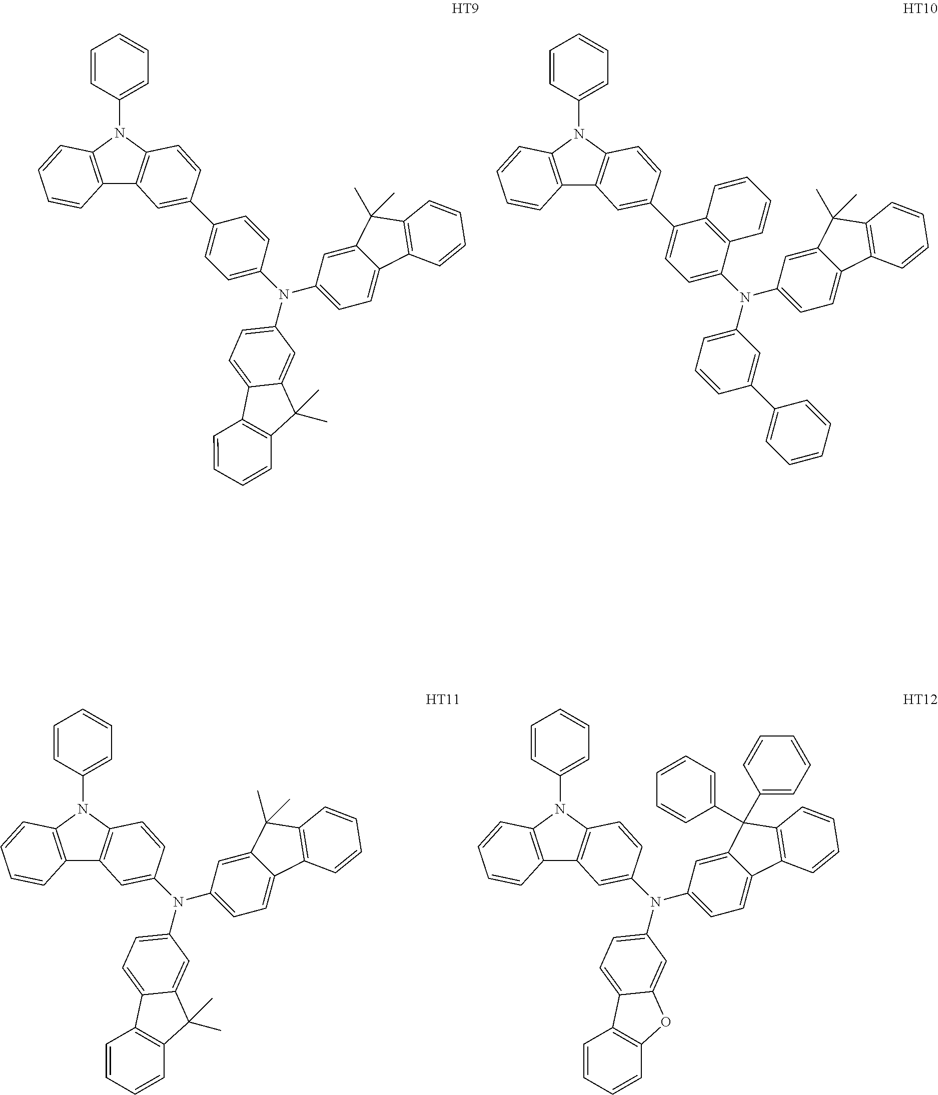 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

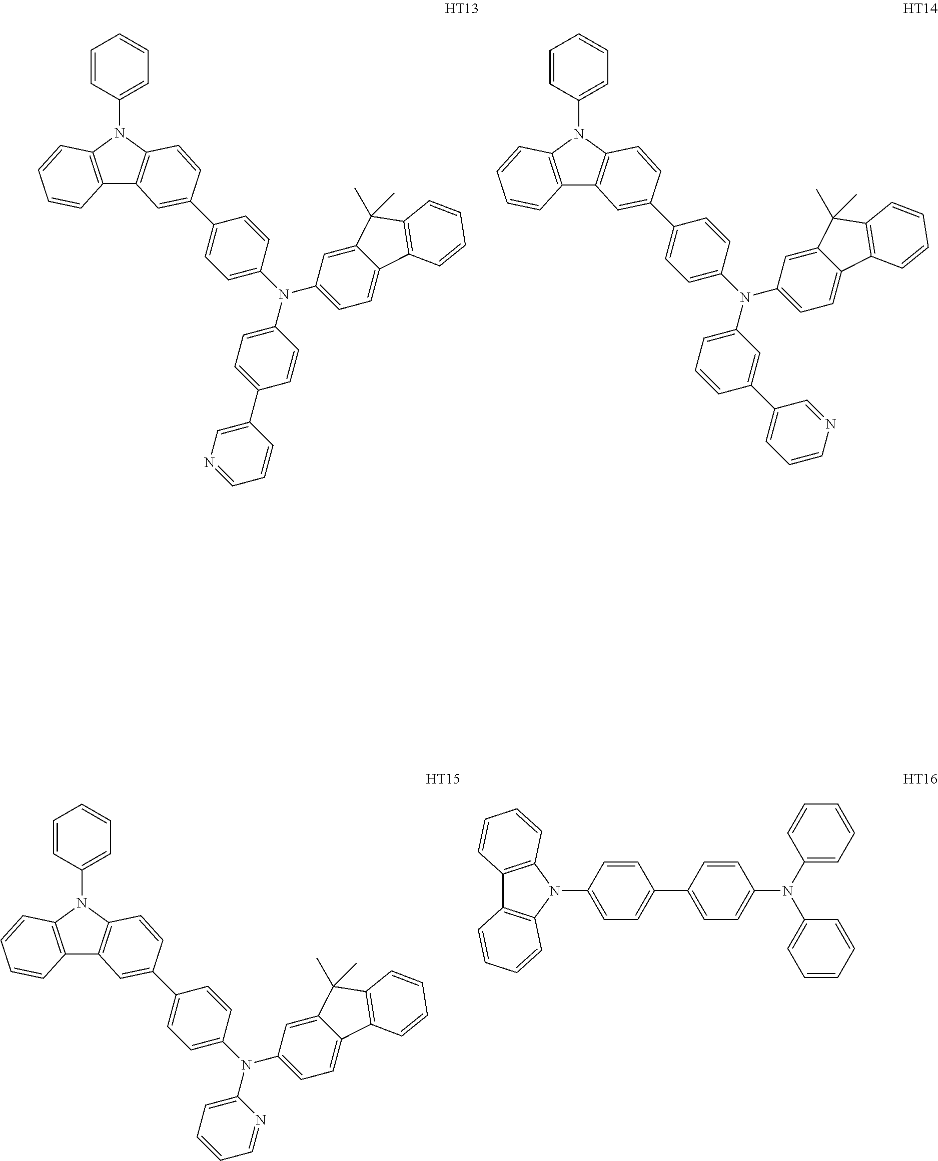 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

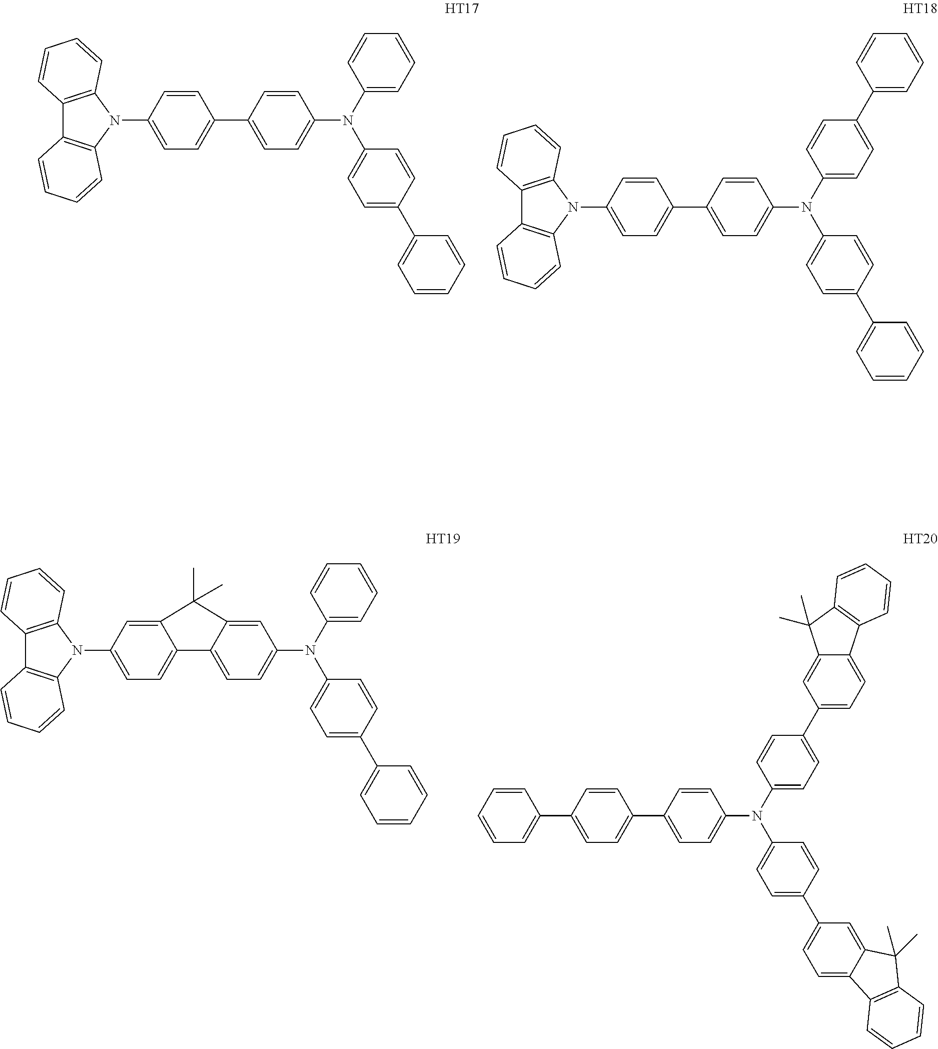

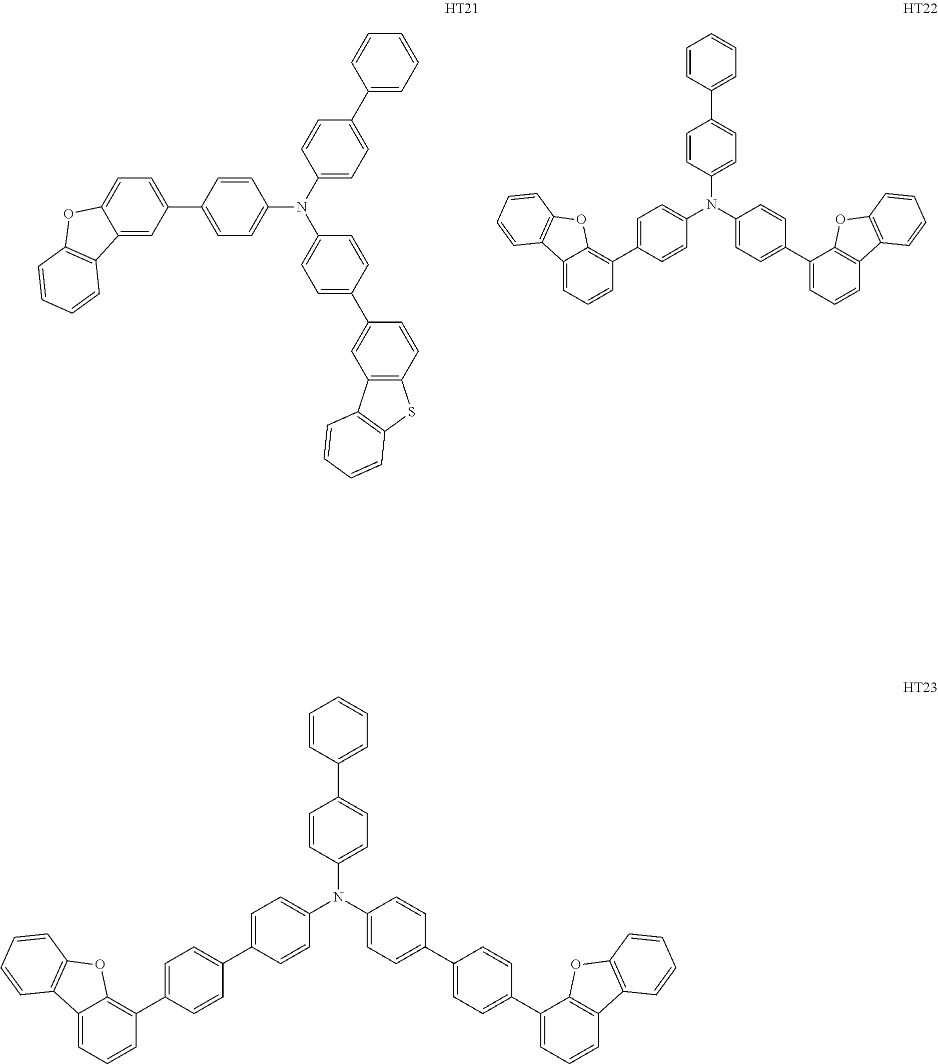 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

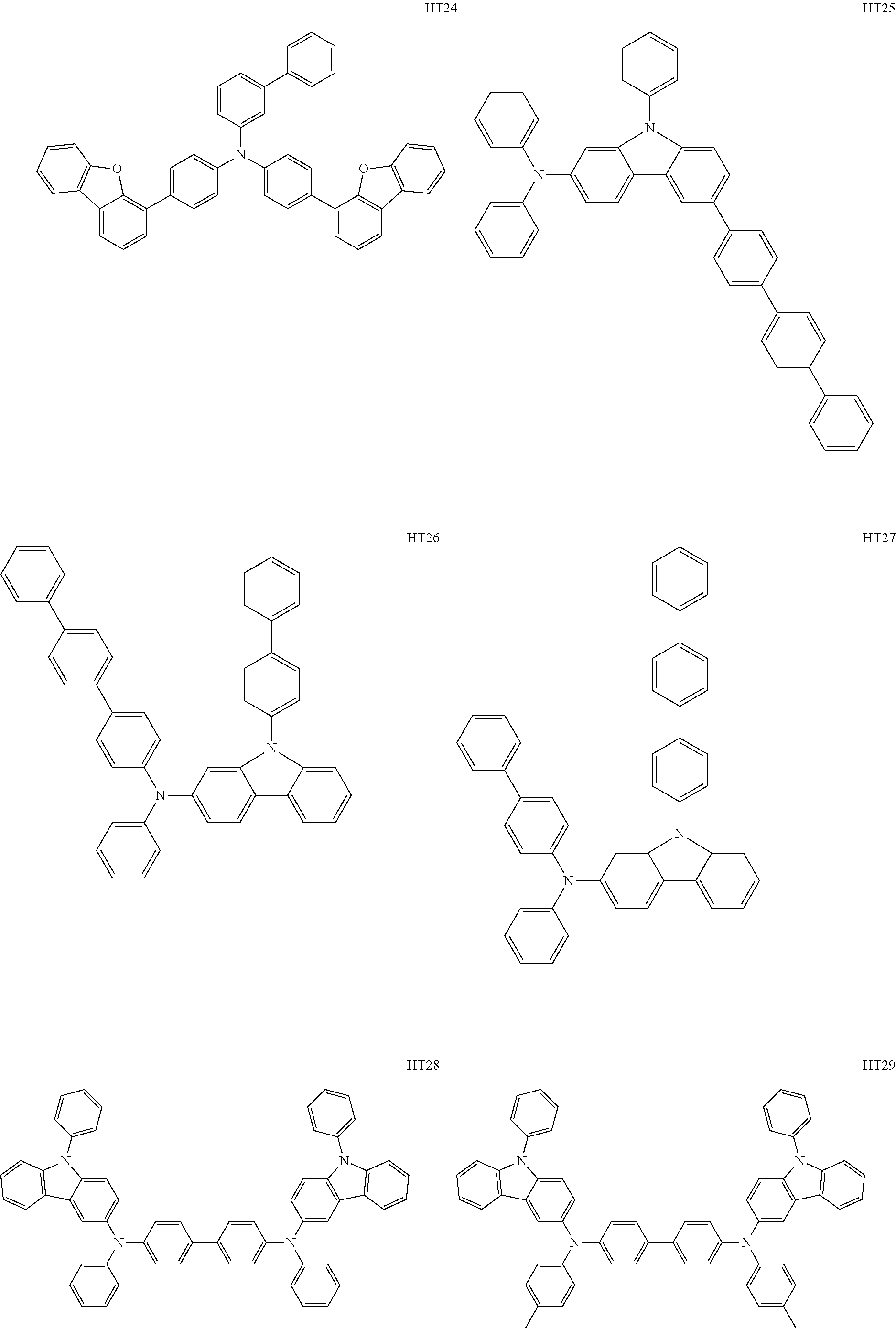

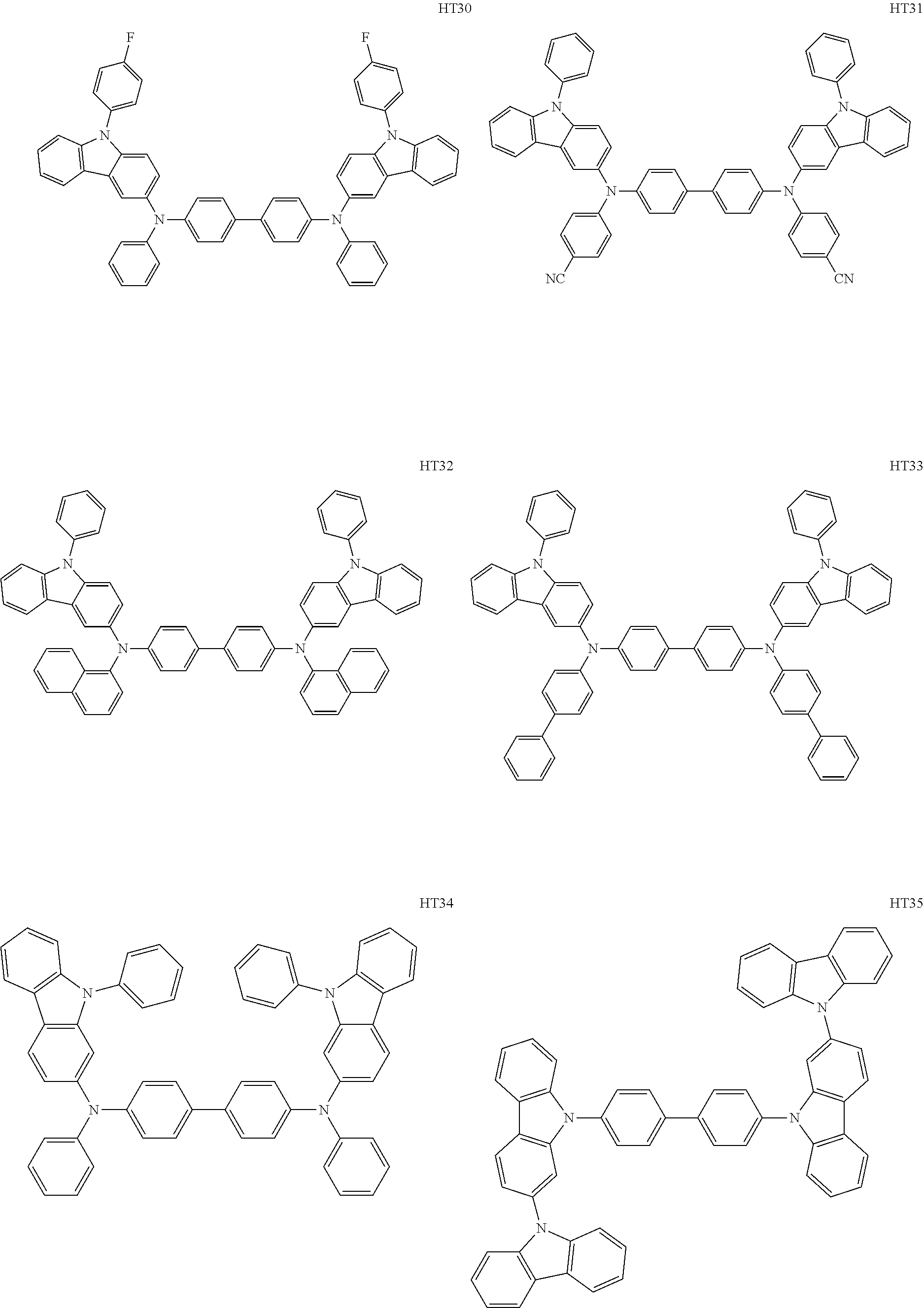

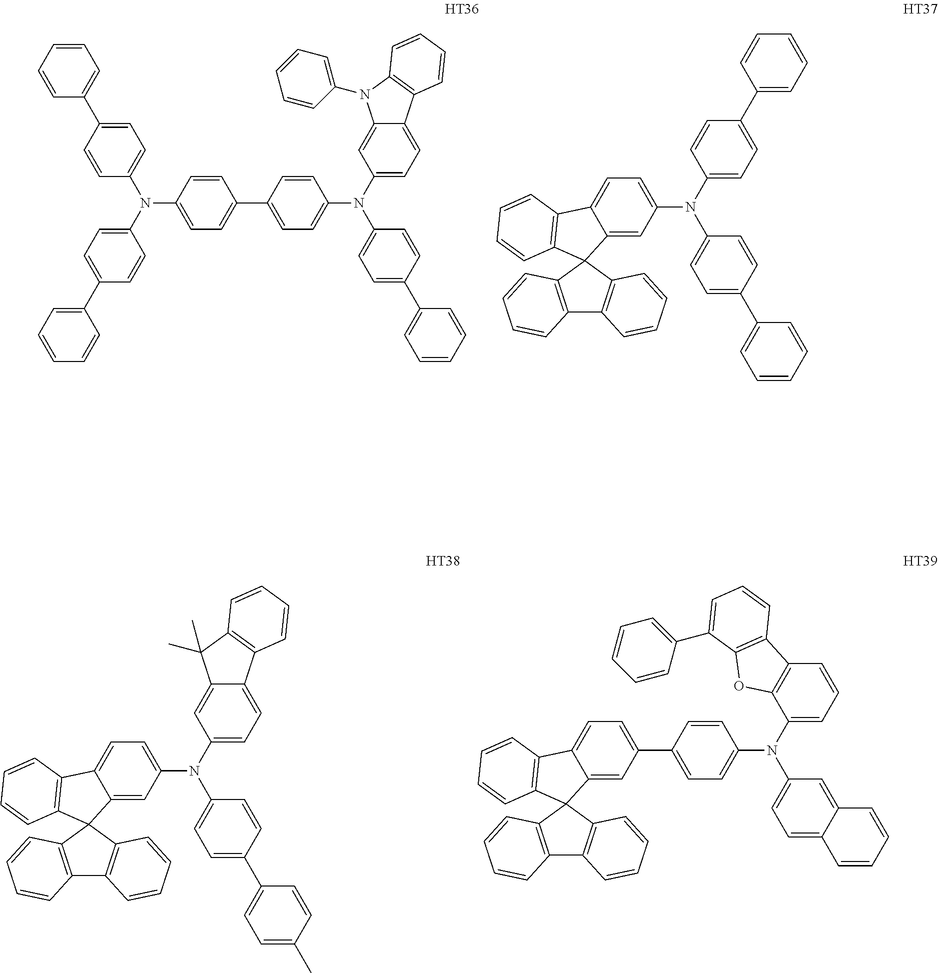 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

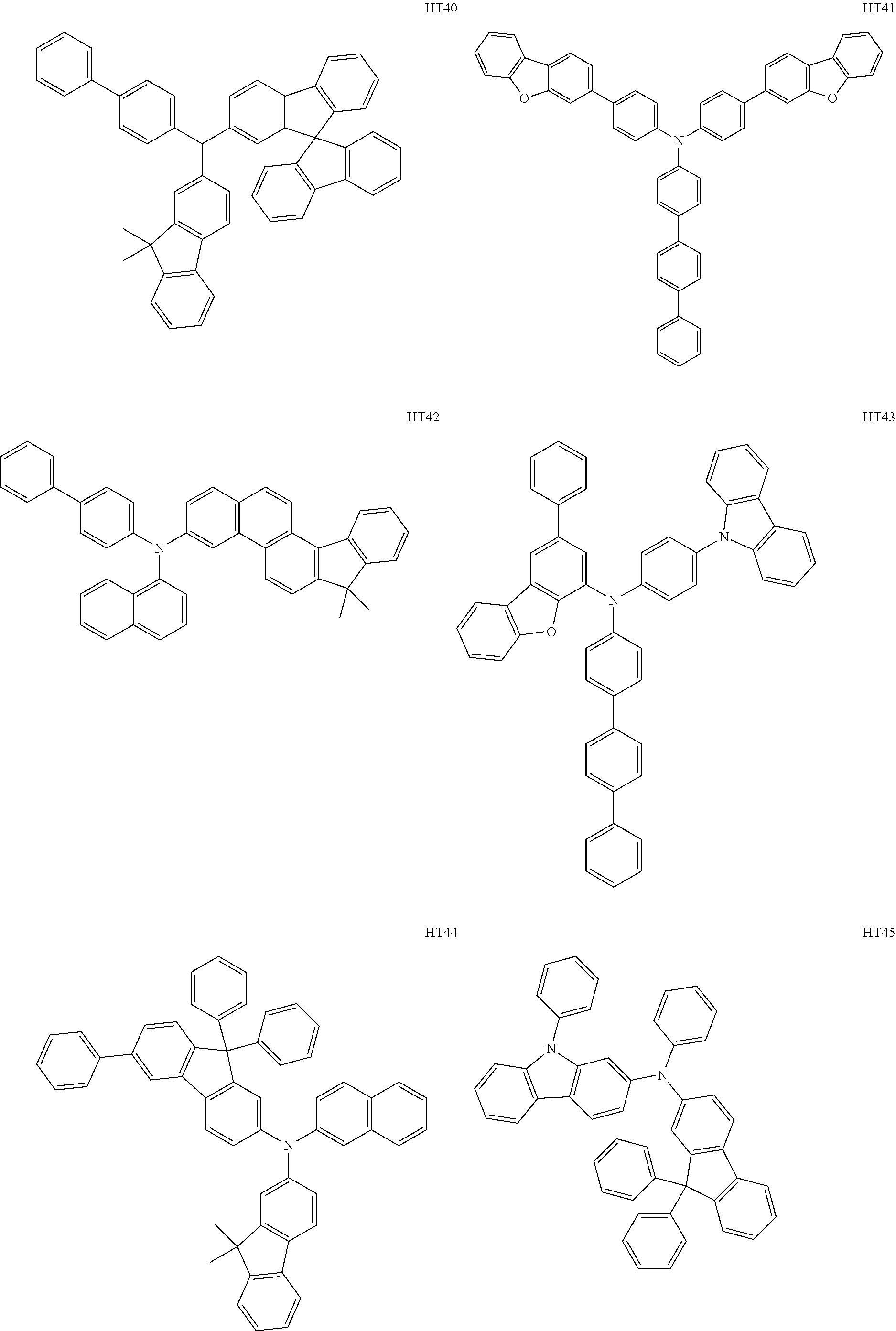

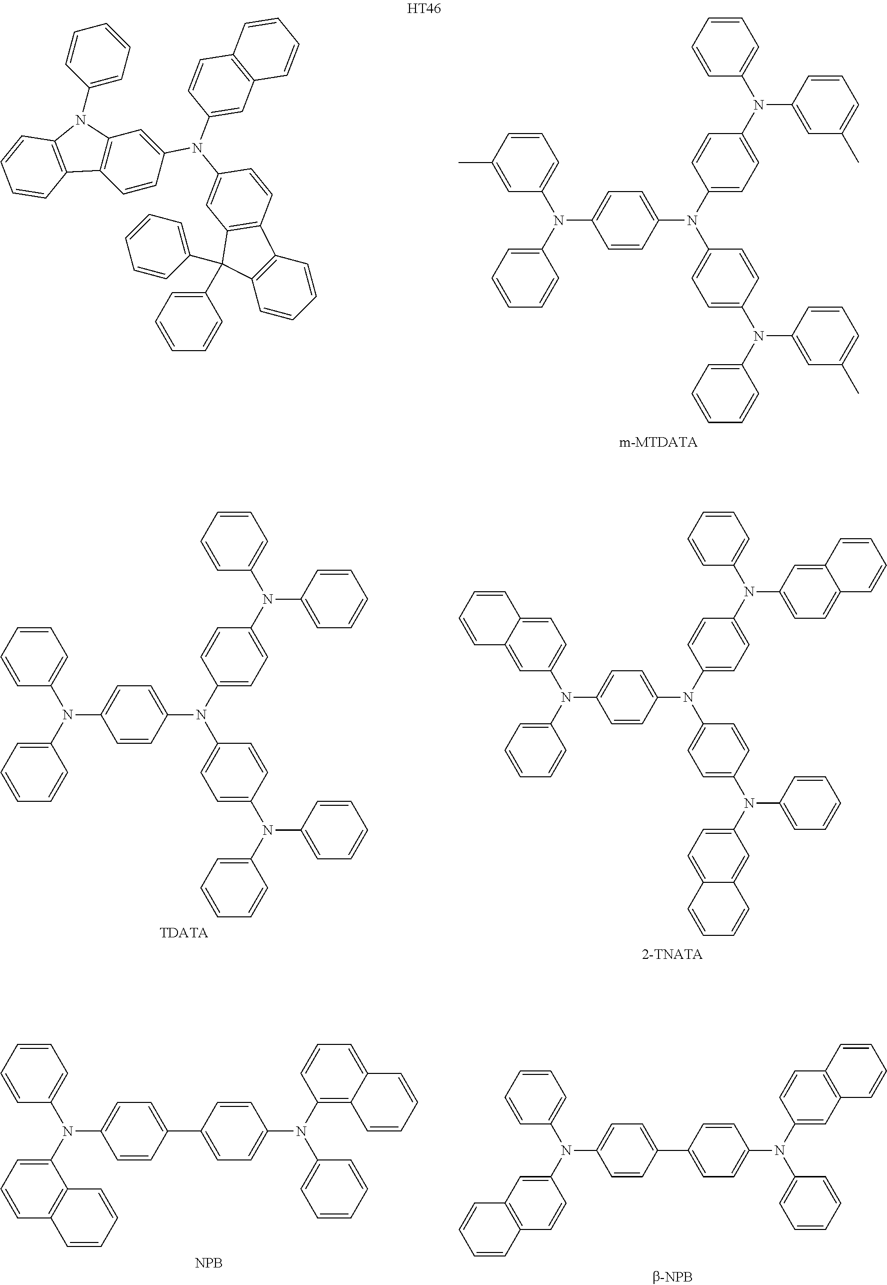

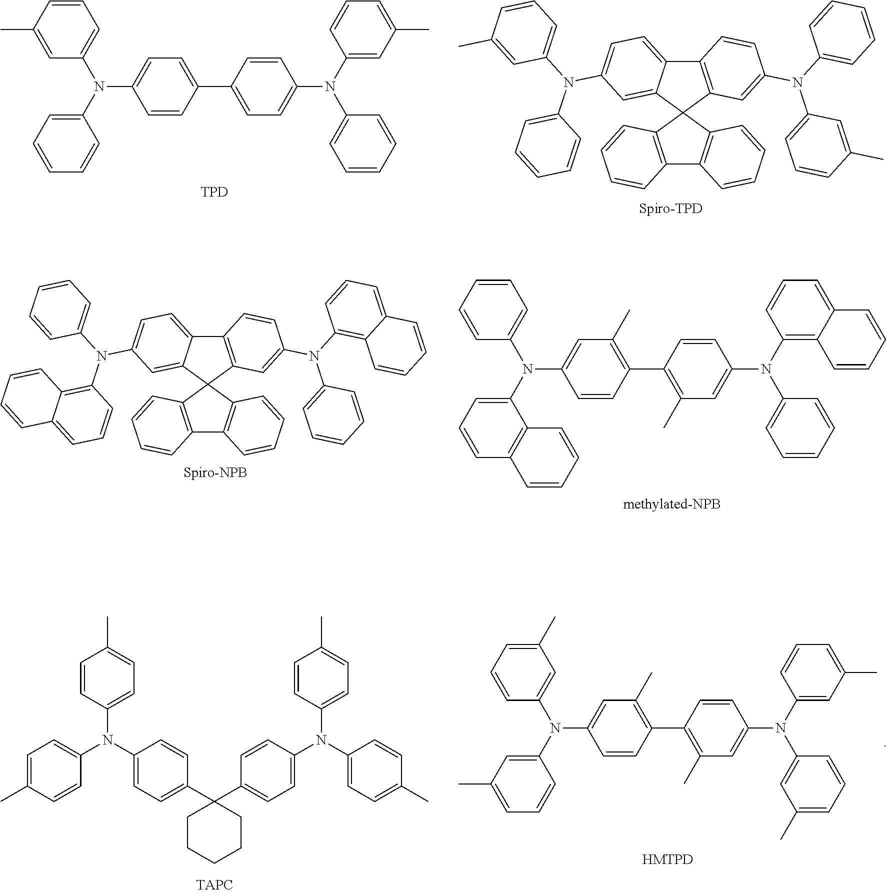

A thickness of the hole transport region may be in a range of about 50 Å to about 10,000 Å, for example, about 100 Å to about 4,000 Å. When the hole transport region includes a hole transport layer, an electron blocking layer or any combination thereof, a thickness of the hole transport layer may be about 50 Å to about 2,000 Å, for example, about 100 Å to about 1,500 Å. When the thicknesses of the hole transport region and the hole transport layer are within the ranges described above, satisfactory hole transportation characteristics may be obtained without a substantial increase in driving voltage.

The emission auxiliary layer may increase light-emission efficiency by compensating for an optical resonance distance of the wavelength of light emitted from the emission layer, and the electron-blocking layer may block or reduce leakage of electrons from the emission layer to the hole transport region. Materials that may be included in the hole transport region may be included in the emission auxiliary layer and the electron-blocking layer.

p-Dopant

The hole transport region may further include, in addition to these materials, a charge-generation material for the improvement of conductive properties. The charge-generation material may be substantially uniformly or non-uniformly dispersed in the hole transport region (for example, in the form of a single layer including (e.g., consisting of) a charge-generation material).

The charge-generation material may be, for example, a p-dopant.

For example, the p-dopant may have a lowest unoccupied molecular orbital (LUMO) energy level of equal to or less than −3.5 eV.

In an embodiment, the p-dopant may include a quinone derivative, a cyano group-containing compound, a compound containing an element EL1 and an element EL2, or any combination thereof.

Examples of the quinone derivative may be or include TCNQ, F4-TCNQ, and/or the like, and

examples of the cyano group-containing compound may be or include HAT-CN, a compound represented by Formula 221, and/or the like:

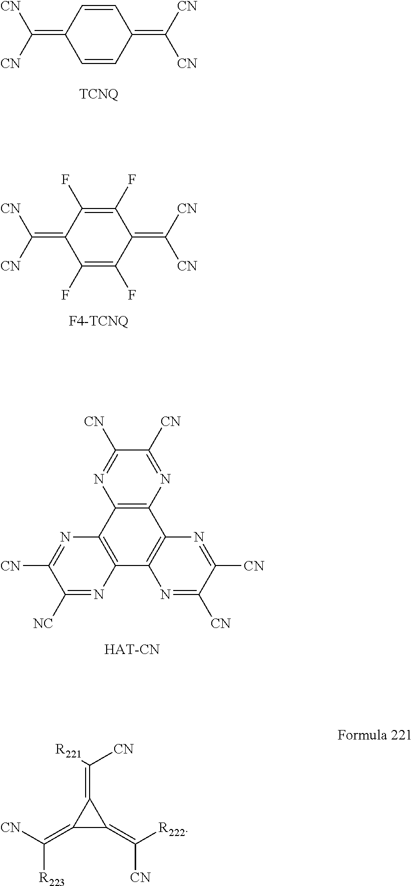

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and

at least one of R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound including the element EL1 and the element EL2, the element EL1 may be a metal, a metalloid, or any combination thereof, and the element EL2 may be a non-metal, a metalloid, or any combination thereof.

Examples of the metal may be or include an alkali metal (for example, lithium (Li), sodium (Na), potassium (K), rubidium (Rb), cesium (Cs), and/or the like); an alkaline earth metal (for example, beryllium (Be), magnesium (Mg), calcium (Ca), strontium (Sr), barium (Ba), and/or the like); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), and/or the like); a post-transition metal (for example, zinc (Zn), indium (In), tin (Sn), and/or the like); and/or a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and/or the like).

Examples of the metalloid may be or include silicon (Si), antimony (Sb), tellurium (Te), and/or the like.

Examples of the non-metal may be or include oxygen (O), halogen (for example, F, Cl, Br, I, and/or the like), and/or the like.

Examples of the compound containing element EL1 and element EL2 may be or include a metal oxide, a metal halide (for example, a metal fluoride, a metal chloride, a metal bromide, a metal iodide, and/or the like), a metalloid halide (for example, a metalloid fluoride, a metalloid chloride, a metalloid bromide, a metalloid iodide, and/or the like), a metal telluride, or any combination thereof.

Examples of the metal oxide may be or include a tungsten oxide (for example, WO, W₂O₃, WO₂, WO₃, W₂O₅, and/or the like), a vanadium oxide (for example, VO, V₂O₃, VO₂, V₂O₅, and/or the like), a molybdenum oxide (MoO, Mo₂O₃, MoO₂, MoO₃, Mo₂O₅, and/or the like), a rhenium oxide (for example, ReO₃, and/or the like), and/or the like.

Examples of the metal halide may be or include an alkali metal halide, an alkaline earth metal halide, a transition metal halide, a post-transition metal halide, a lanthanide metal halide, and/or the like.

Examples of the alkali metal halide may be or include LiF, NaF, KF, RbF, CsF, LiCl, NaCl, KCl, RbCl, CsCl, LiBr, NaBr, KBr, RbBr, CsBr, LiI, NaI, KI, RbI, CsI, and/or the like.

Examples of the alkaline earth metal halide may be or include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, Bale, and/or the like.

Examples of the transition metal halide may be or include a titanium halide (for example, TiF₄, TiCl₄, TiBr₄, TiI₄, and/or the like), a zirconium halide (for example, ZrF₄, ZrCl₄, ZrBr₄, ZrI₄, and/or the like), a hafnium halide (for example, Hf F₄, HfCl₄, HfBr₄, Hf₁₄, and/or the like), a vanadium halide (for example, VF₃, VCl₃, VBr₃, V₁₃, and/or the like), a niobium halide (for example, NbF₃, NbCl₃, NbBr₃, NbI₃, and/or the like), a tantalum halide (for example, TaF₃, TaCl₃, TaBr₃, TaI₃, and/or the like), a chromium halide (for example, CrF₃, CrCl₃, CrBr₃, Cr₁₃, and/or the like), a molybdenum halide (for example, MoF₃, MoCl₃, MoBr₃, Mo₁₃, and/or the like), a tungsten halide (for example, WF₃, WCl₃, WBr₃, WI₃, and/or the like), a manganese halide (for example, MnF₂, MnCl₂, MnBr₂, MnI₂, and/or the like), a technetium halide (for example, TcF₂, TcCl₂, TcBr₂, Tcl₂, and/or the like), a rhenium halide (for example, ReF₂, ReCl₂, ReBr₂, Rel₂, and/or the like), an iron halide (for example, FeF₂, FeCl₂, FeBr₂, Felt, and/or the like), a ruthenium halide (for example, RuF₂, RuCl₂, RuBr₂, Rule, and/or the like), an osmium halide (for example, OsF₂, OsCl₂, OsBr₂, OsI₂, and/or the like), a cobalt halide (for example, CoF₂, CoCl₂, CoBr₂, Cole, and/or the like), a rhodium halide (for example, RhF₂, RhCl₂, RhBr₂, Rhl₂, and/or the like), an iridium halide (for example, IrF₂, IrCl₂, IrBr₂, IrI₂, and/or the like), a nickel halide (for example, NiF₂, NiCl₂, NiBr₂, Nile, and/or the like), a palladium halide (for example, PdF₂, PdCl₂, PdBr₂, Pdl₂, and/or the like), a platinum halide (for example, PtF₂, PtCl₂, PtBr₂, Ptl₂, and/or the like), a copper halide (for example, CuF, CuCl, CuBr, CuI, and/or the like), a silver halide (for example, AgF, AgCl, AgBr, AgI, and/or the like), a gold halide (for example, AuF, AuCl, AuBr, AuI, and/or the like), and/or the like.

Examples of the post-transition metal halide may be or include a zinc halide (for example, ZnF₂, ZnCl₂, ZnBr₂, ZnI₂, and/or the like), an indium halide (for example, InI₃ and/or the like), a tin halide (for example, SnI₂, and/or the like), and/or the like.

Examples of the lanthanide metal halide may be or include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, Sm₁₃, and/or the like.

Examples of the metalloid halide may be or include an antimony halide (for example, SbCl₅ and/or the like) and/or the like.

Examples of the metal telluride may be or include an alkali metal telluride (for example, Li₂Te, Na₂Te, K₂Te, Rb₂Te, Cs₂Te, and/or the like), an alkaline earth metal telluride (for example, BeTe, MgTe, CaTe, SrTe, BaTe, and/or the like), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and/or the like), a post-transition metal telluride (for example, ZnTe, and/or the like), a lanthanide metal telluride (for example, LaTe, CeTe, PrTe, NdTe, PmTe, EuTe, GdTe, TbTe, DyTe, HoTe, ErTe, TmTe, YbTe, LuTe, and/or the like), and/or the like.

### Emission Layer in Interlayer 130

When the light-emitting device 10 is a full-color light-emitting device, the emission layer may be patterned into a red emission layer, a green emission layer, and/or a blue emission layer, according to a sub-pixel. In an embodiment, the emission layer may have a stacked structure of two or more layers of a red emission layer, a green emission layer, and a blue emission layer, in which the two or more layers may contact each other or may be separated from each other, and may together emit white light. In one or more embodiments, the emission layer may a structure in which two or more materials of a red light-emitting material, a green light-emitting material, and a blue light-emitting material are mixed with each other in a single layer to emit white light.

In an embodiment, the emission layer may include a host and a dopant. The host and the dopant may each independently be the same as described above.

In the emission layer, an amount of the dopant may be in a range of about 0.01 parts by weight to about 15 parts by weight based on 100 parts by weight of the total hosts.

In an embodiment, a weight ratio of the HTH1 to the ETH1 may be, for example, in a range of about 1:9 to about 9:1. In one or more embodiments, the weight ratio of the HTH1 to the ETH1 may be in a range of about 2:8 to about 8:2, about 3:7 to about 7:3, or about 4:6 to about 6:4, or may be about 5:5.

In an embodiment, a weight ratio of the HTH2 to the ETH2 may be, for example, in a range of about 1:9 to about 9:1. In one or more embodiments, the weight ratio of the HTH2 to the ETH2 may be in a range of about 2:8 to about 8:2, about 3:7 to about 7:3, or about 4:6 to about 6:4, or may be about 5:5.

For example, the weight ratio of the HTH1 to the ETH1 may not be identical to the weight ratio of the HTH2 to the ETH2.

When the weight ratios of the HT host and the ET host are within the ranges above, the hole transport may be in a desirable balance with the electron transport.

A thickness of the emission layer 4 may be in a range of about 100 Å to about 1,000 Å, for example, about 200 Å to about 600 Å. When the thickness of the emission layer 4 is within these ranges, excellent or suitable luminescence characteristics may be obtained without a substantial increase in driving voltage.

### Quantum Dots

The electronic apparatus may include quantum dots. For example, the electronic apparatus may include a color conversion layer, and the color conversion layer may include quantum dots.

The term “quantum dots” as used herein refers to crystals of a semiconductor compound, and may include any material capable of emitting light of one or more suitable emission wavelengths according to the size of the crystals.

A diameter of the quantum dots may be, for example, in a range of about 1 nm to about 10 nm.

The quantum dots may be synthesized by a wet chemical process, a metal organic chemical vapor deposition process, a molecular beam epitaxy process, or any process similar thereto.

The wet chemical process is a method that includes mixing a precursor material with an organic solvent and then growing quantum dot particle crystals. As the crystals grow, the organic solvent naturally acts as a dispersant coordinated on the surface of the quantum dot crystals to control or select the growth of the quantum dot particles through a lower cost process that is easier than vapor deposition methods (such as metal organic chemical vapor deposition (MOCVD) and/or molecular beam epitaxy (MBE)).

The quantum dots may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Examples of the Group II-VI semiconductor compound may be or include: a binary compound (such as CdSe, CdTe, ZnS, ZnSe, ZnTe, ZnO, HgS, HgSe, HgTe, MgSe, MgS, and/or the like); a ternary compound (such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and/or the like); a quaternary compound (such as CdZnSeS, CdZnSeTe, CdZnSTe, CdHgSeS, CdHgSeTe, CdHgSTe, HgZnSeS, HgZnSeTe, HgZnSTe, and/or the like); or any combination thereof.

Examples of the Group III-V semiconductor compound may be or include: a binary compound (such as GaN, GaP, GaAs, GaSb, AlN, AlP, AlAs, AlSb, InN, InP,

InAs, InSb, and/or the like); a ternary compound (such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, and/or the like); a quaternary compound (such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and/or the like); or any combination thereof. The Group III-V semiconductor compound may further include a Group II element. Examples of the Group III-V semiconductor compound further including the Group II element may be or include InZnP, InGaZnP, InAlZnP, and/or the like.

Examples of the Group III-VI semiconductor compound may include: a binary compound (such as GaS, GaSe, Ga₂Se₃, GaTe, InS, InSe, In₂Se₃, and/or InTe); a ternary compound (such as InGaS₃ and/or InGaSe₃); or any combination thereof.

Examples of the Group I-III-VI semiconductor compound may be or include: a ternary compound (such as AgInS, AgInS₂, CuInS, CuInS₂, CuGaO₂, AgGaO₂, AgAlO₂, and/or the like); or any combination thereof.

Examples of the Group IV-VI semiconductor compound may be or include: a binary compound (such as SnS, SnSe, SnTe, PbS, PbSe, PbTe, and/or the like); a ternary compound (such as SnSeS, SnSeTe, SnSTe, PbSeS, PbSeTe, PbSTe, SnPbS, SnPbSe, SnPbTe, and/or the like); a quaternary compound (such as SnPbSSe, SnPbSeTe, SnPbSTe, and/or the like); or any combination thereof.

The Group IV element or compound may include: a single element compound (such as Si, Ge, and/or the like); a binary compound (such as SiC, SiGe, and/or the like); or any combination thereof.

Each element included in a multi-element compound (such as the binary compound, the ternary compound, and/or the quaternary compound) may exist in a particle thereof at a substantially uniform concentration (e.g., spatial distribution) or non-substantially uniform concentration.

The quantum dot may have a single structure or a dual core-shell structure. In the case of the quantum dot having a single structure, a concentration of each element included in the corresponding quantum dot may be substantially uniform. For example, a material included in the core and a material included in the shell may be different from each other.

The shell of the quantum dot may act as a protective layer that prevents or reduces chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases toward the center of the core.

Examples of the shell of the quantum dot may be or include a metal oxide, a metalloid oxide, a non-metal oxide, a semiconductor compound, or any combination thereof. Examples of the metal oxide, metalloid oxide, or non-metal oxide may be or include: a binary compound (such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, and/or the like); a ternary compound (such as MgAl₂O₄, CoFe₂O₄, NiFe₂O₄, CoMn₂O₄, and/or the like); or any combination thereof. Examples of the semiconductor compound may be or include: as described herein, a Group II-VI semiconductor compound; a Group III-V semiconductor compound; a Group III-VI semiconductor compound; a Group I-III-VI semiconductor compound; a Group IV-VI semiconductor compound; or any combination thereof. Examples of the semiconductor compound may be or include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of an emission wavelength spectrum of the quantum dots may be equal to or less than about 45 nm, for example, equal to or less than about 40 nm, and for example, equal to or less than about 30 nm, and within these ranges, color purity or color reproducibility may be improved. Because the light emitted through the quantum dots is emitted in all directions, the wide viewing angle may be improved.

In some embodiments, the quantum dot may be or include spherical, pyramidal, multi-arm, and/or cubic nanoparticles, nanotubes, nanowires, nanofibers, and/or nanoplate particles.

Because the energy band gap may be adjusted by controlling the size of the quantum dots, light having one or more suitable wavelength bands may be obtained from the emission layer including the quantum dots. Accordingly, by utilizing quantum dots of different sizes, a light-emitting device that emits light of various suitable wavelengths may be implemented. For example, the size of the quantum dots may be selected to emit red light, green light, and/or blue light. In some embodiments, the size of the quantum dots may be selected to emit white light by combining light of one or more suitable colors.

### Electron Transport Region in Interlayer 130

The electron transport region may have i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The electron transport region may include a hole-blocking layer, an electron transport layer, an electron injection layer, or any combination thereof.

For example, the electron transport region may have an electron transport layer/electron injection layer structure, a hole blocking layer/electron transport layer/electron injection layer structure, and/or the like, the constituting layers of each structure being sequentially stacked from the emission layer.

In an embodiment, the electron transport region (for example, the hole-blocking layer, or the electron transport layer in the electron transport region) may include a metal-free compound including at least one π electron-deficient nitrogen-containing C₁-C₆₀ cyclic group.

In one or more embodiments, the electron transport region may include a compound represented by Formula 601:

[Ar₆₀₁]ₓₑ₁₁-[(L₆₀₁)ₓₑ₁-R₆₀₁]ₓₑ₂₁, Formula 601

wherein, in Formula 601,

Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂ (Q₆₀₁), or —P(═O)(Q₆₀₁)(Q₆₀₂),

Q₆₀₁ to Q₆₀₃ may each independently be the same as described in connection with Q₁,

xe21 may be 1, 2, 3, 4, or 5, and

at least one of Ar₆₀₁, L₆₀₁, and R₆₀₁ may each independently be air electron-deficient nitrogen-containing C₁-C₆₀ cyclic group unsubstituted or substituted with at least one R₁₀ₐ.

In an embodiment, when xe11 in Formula 601 is 2 or more, two or more Ar₆₀₁ (s) may be linked to each other via a single bond.

In an embodiment, Ar₆₀₁ in Formula 601 may be a substituted or unsubstituted anthracene group.

In one or more embodiments, the electron transport region may include a compound represented by Formula 601-1:

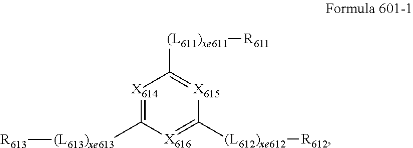

wherein, in Formula 601-1,

X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,

L₆₁₁ to L₆₁₃ may each independently be the same as described in connection with L₆₀₁,

xe611 to xe613 may each independently be the same as described in connection with xe1,

R₆₁₁ to R₆₁₃ may each independently be the same as described in connection with R₆₀₁, and

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

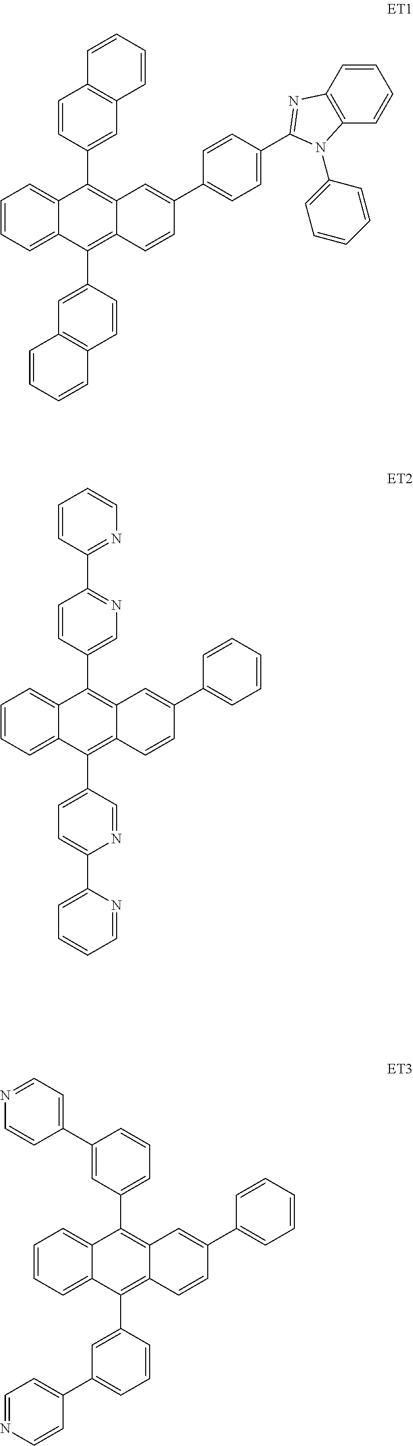 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

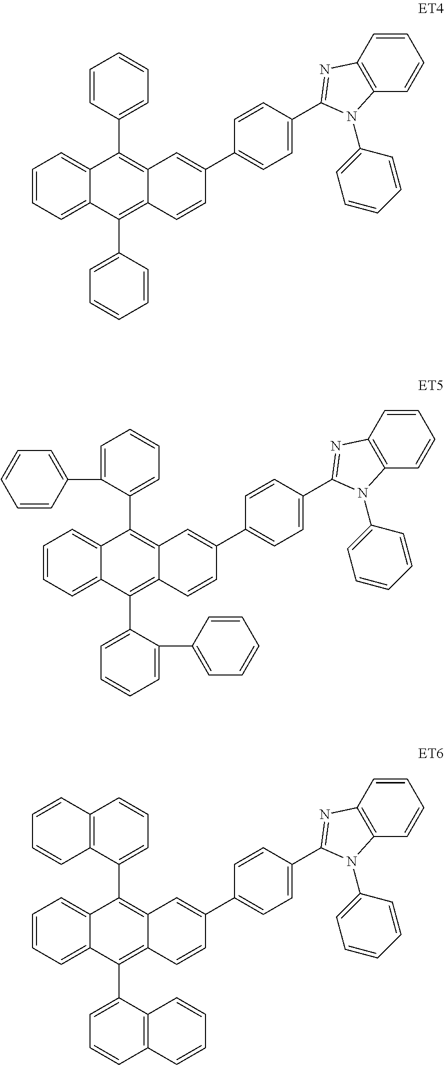 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

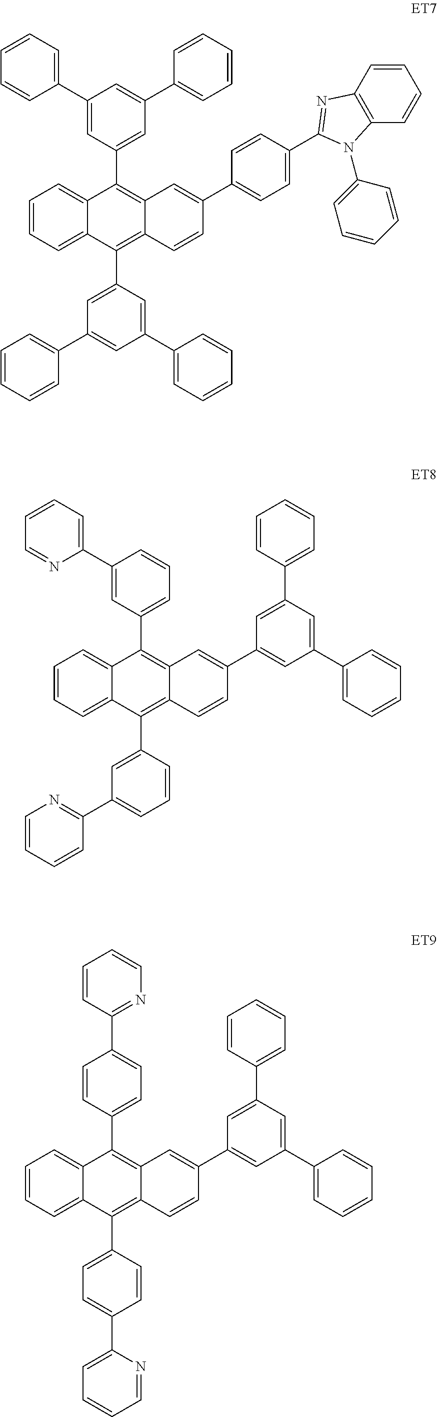 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

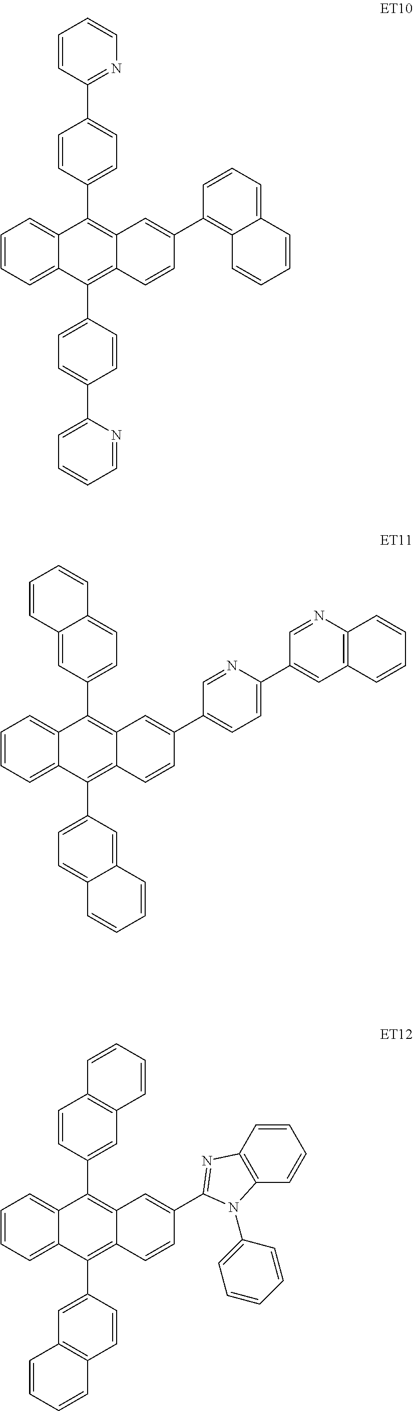 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

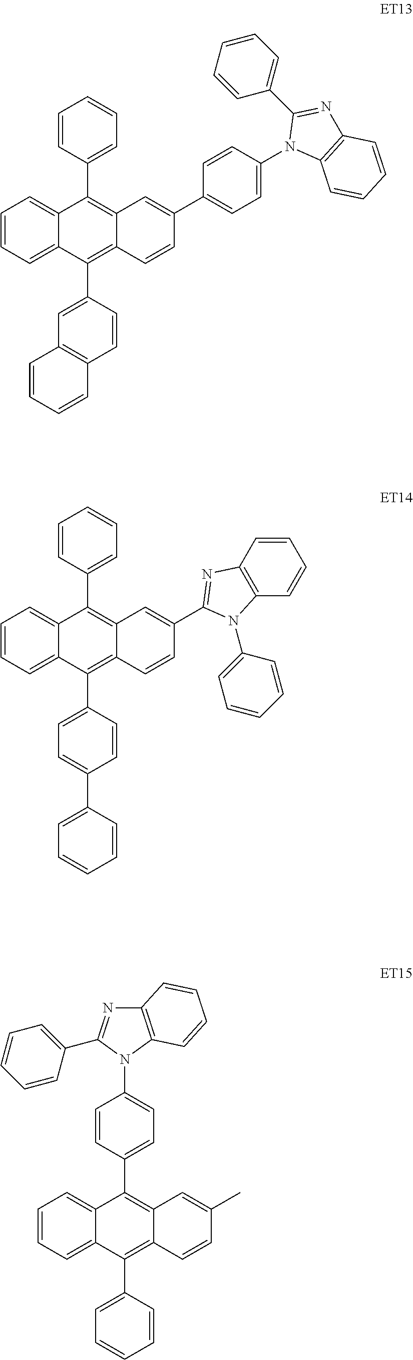 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

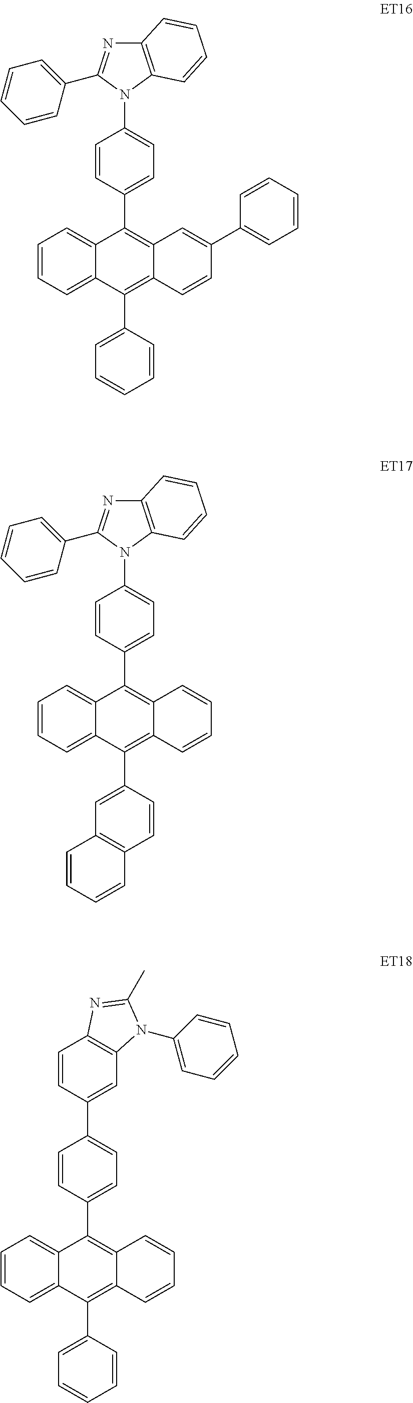 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

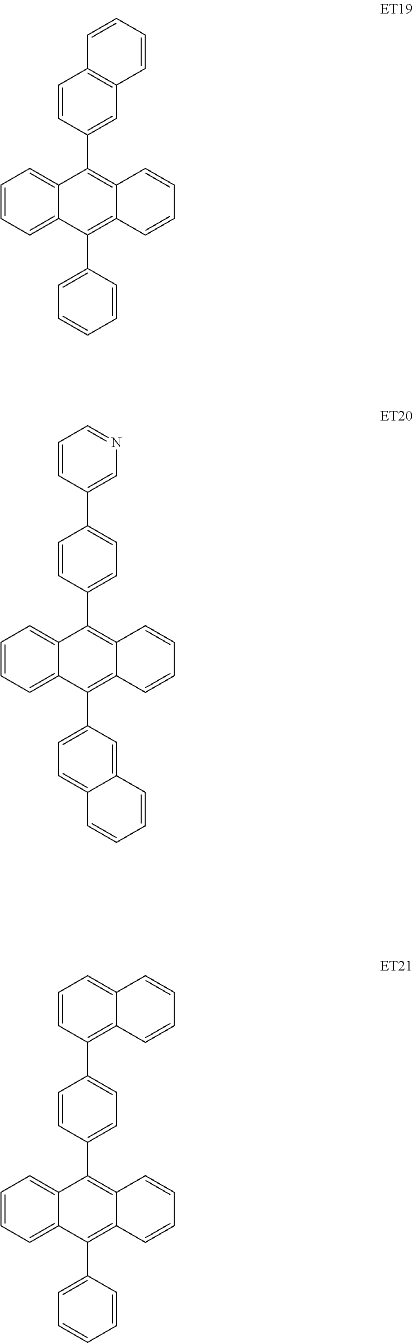

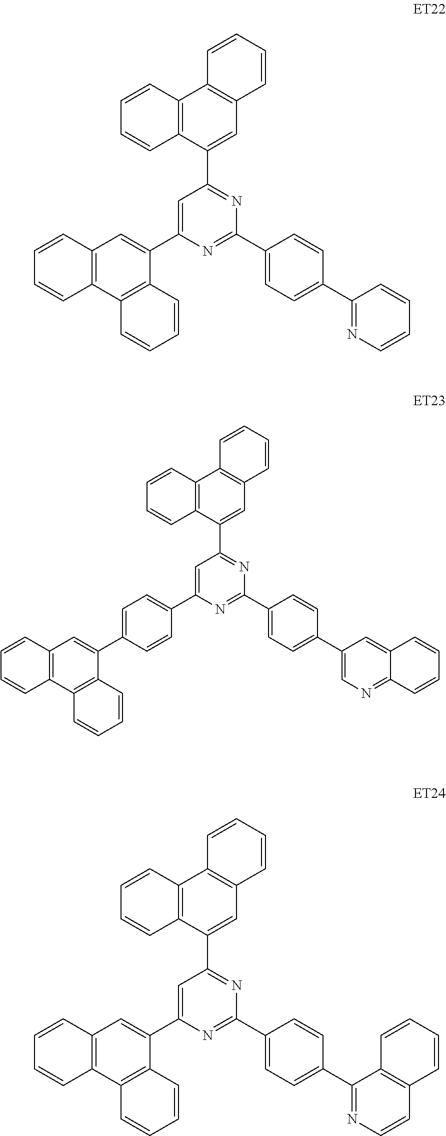 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

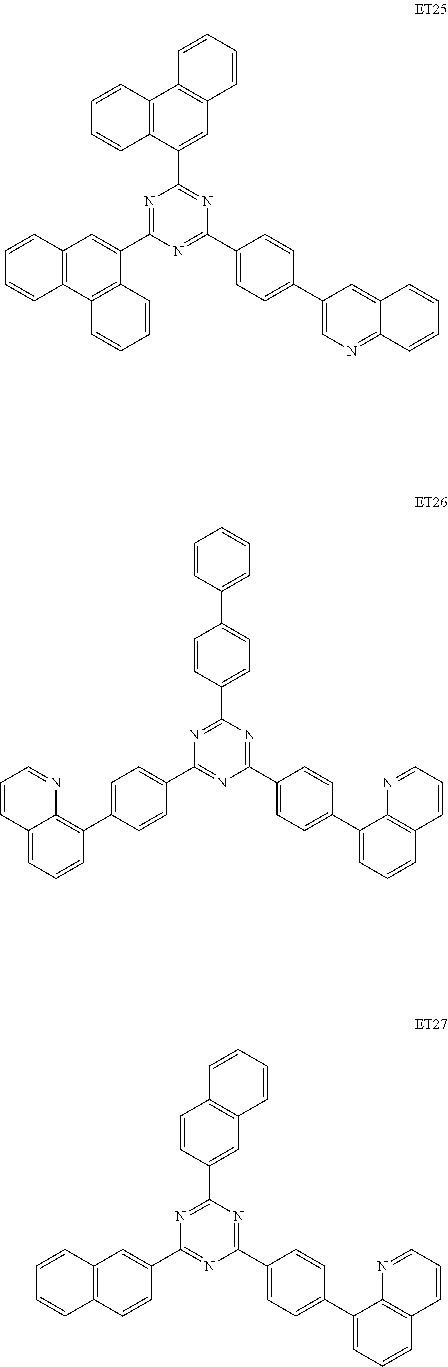 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

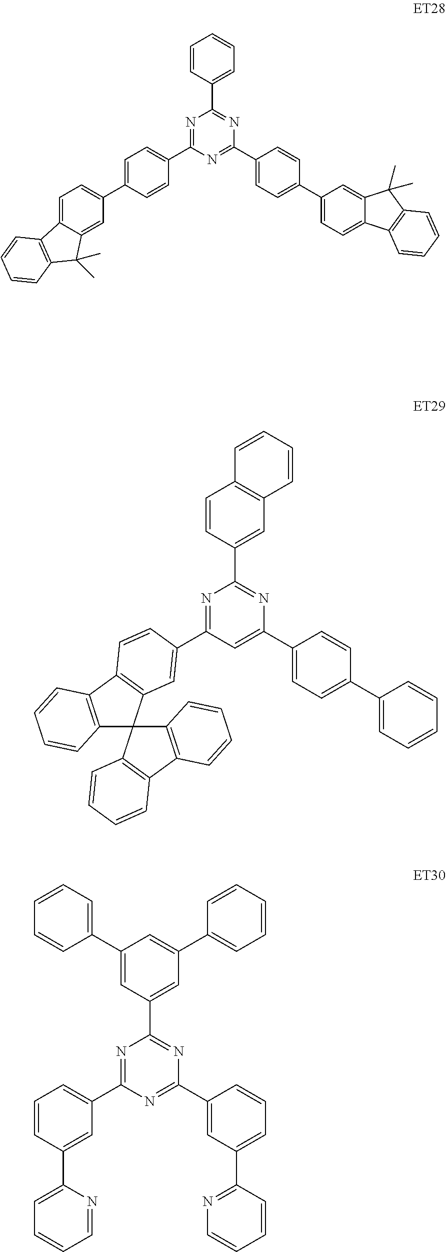 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

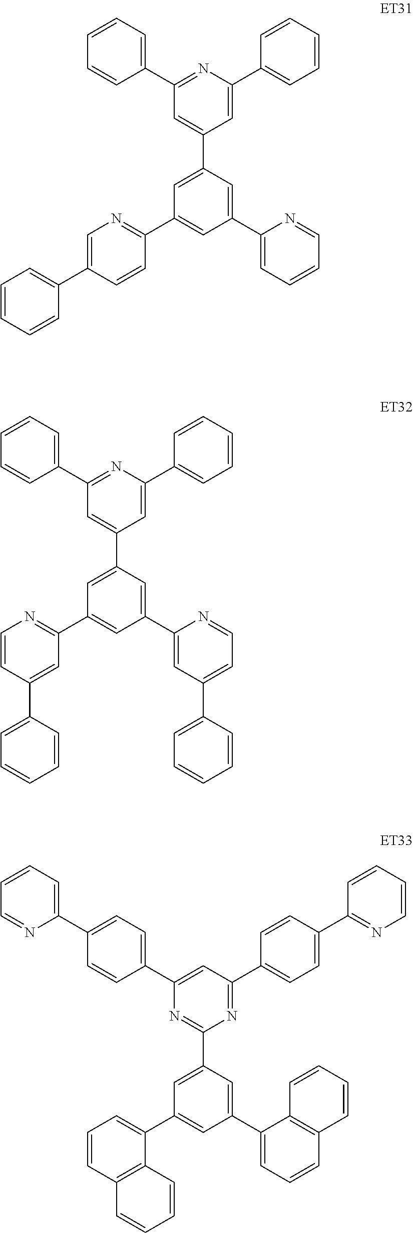 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

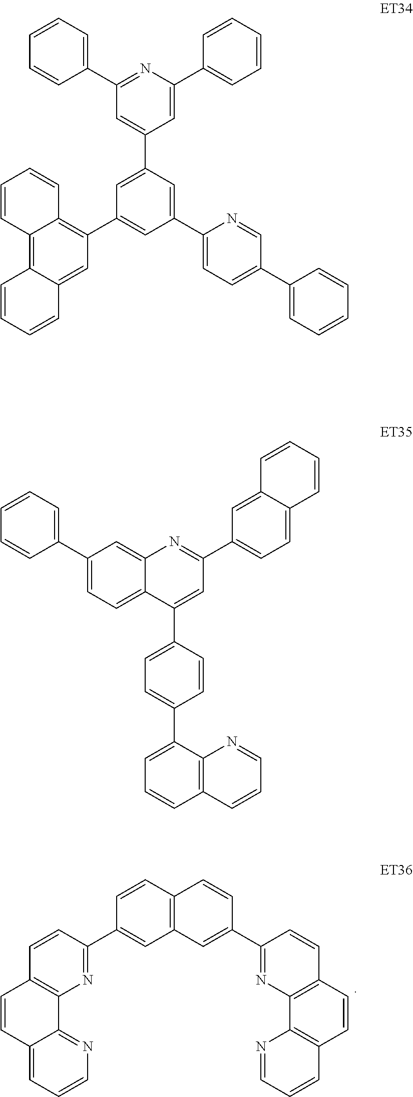 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

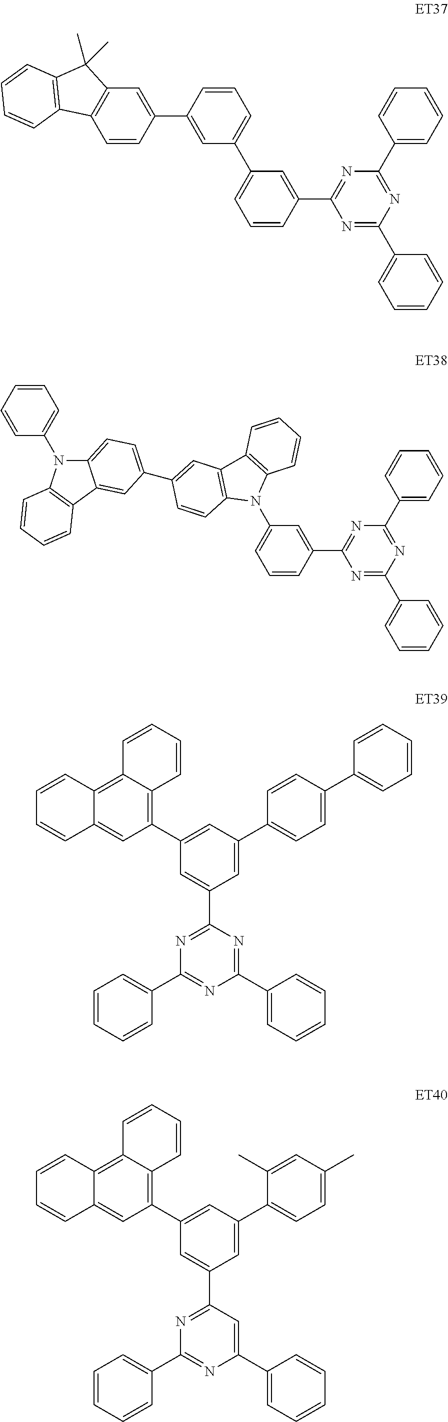 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

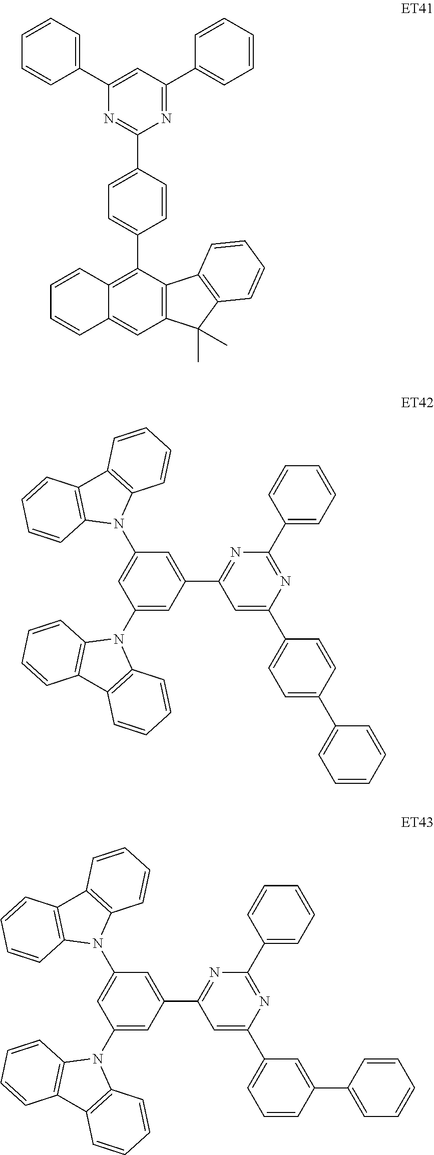 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

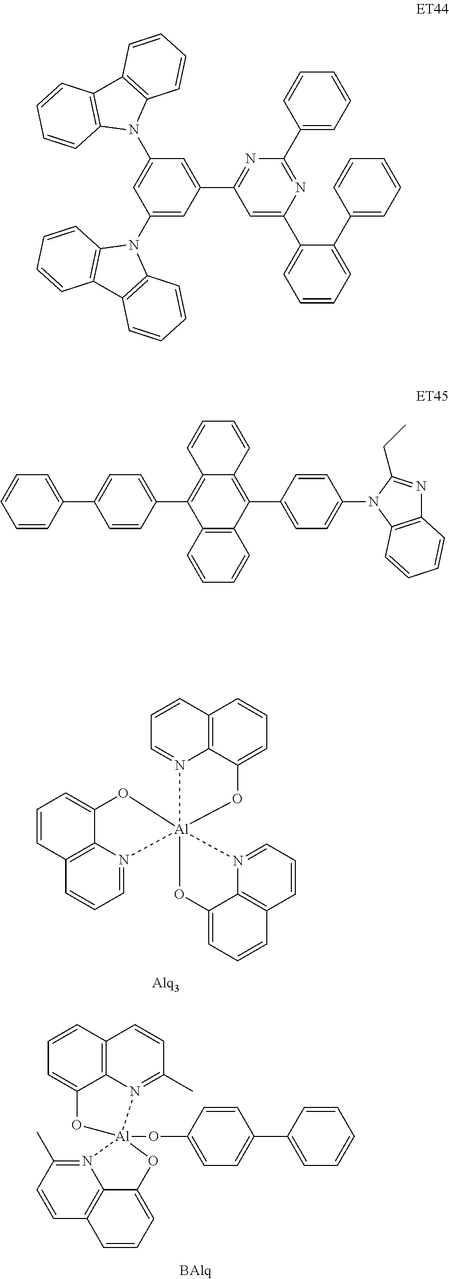

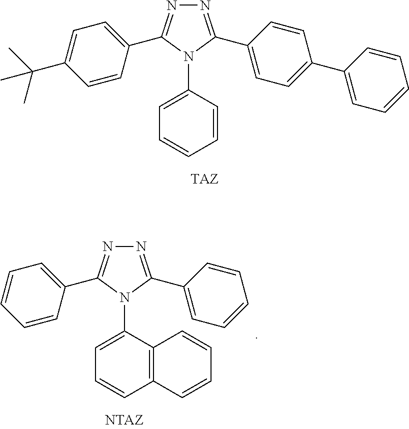 [SMILES: CC(C)(C)c1ccc(-c2nnc(-c3ccc(-c4ccccc4)cc3)n2-c2ccccc2)cc1 ; c1ccc(-c2nnc(-c3ccccc3)n2-c2cccc3ccccc23)cc1]

A thickness of the electron transport region may be in a range of about 100 Å to about 5,000 Å, for example, about 160 Å to about 4,000 Å. When the electron transport region includes the hole blocking layer, the electron transport layer, or any combination thereof, a thickness of the hole blocking layer or electron transport layer may each independently be about 20 Å to about 1,000 Å, for example, about 30 Å to about 300 Å, and the thickness of the electron transport layer may be about 100 Å to about 1,000 Å, for example, about 150 Å to about 500 Å. When the thicknesses of the hole blocking layer and/or the electron transport layer are within these ranges, satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. The metal ion of an alkali metal complex may be a Li ion, a Na ion, a K ion, a Rb ion, or a Cs ion, and the metal ion of an alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a Li complex. The Li complex may include, for example, Compound ET-D1 (LiQ) or ET-D2:

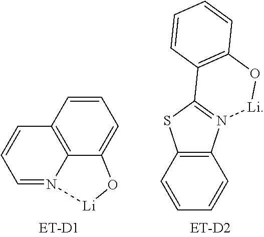

The electron transport region may include an electron injection layer to facilitate injection of electrons from the second electrode 150. The electron injection layer may directly contact the second electrode 150.

The electron injection layer may have: i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include lithium (Li), sodium (Na), potassium (K), rubidium (Rb), cesium (Cs), or any combination thereof. The alkaline earth metal may include magnesium (Mg), calcium (Ca), strontium (Sr), barium (Ba), or any combination thereof. The rare earth metal may include scandium (Sc), yttrium (Y), cerium (Ce), terbium (Tb), ytterbium (Yb), gadolinium (Gd), or any combination thereof.

The alkali metal-containing compound, the alkaline earth metal-containing compound, and the rare earth metal-containing compound may be or include oxides, halides (for example, fluorides, chlorides, bromides, iodides, and/or the like), and/or tellurides of the alkali metal, the alkaline earth metal, and the rare earth metal, respectively.

The alkali metal-containing compound may include alkali metal oxides (such as Li₂O, Cs₂O, K₂O, and/or the like), alkali metal halides (such as LiF, NaF, CsF, KF, LiI, NaI, CsI, KI, and/or the like), or any combination thereof. The alkaline earth metal-containing compound may include an alkaline earth metal compound (such as BaO, SrO, CaO, BaₓSr₁₋ₓO (wherein x is a real number satisfying the condition of 0<x<₁), BaₓCa₁₋ₓO (wherein x is a real number satisfying the condition of 0<x<₁), and/or the like). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. For example, the rare earth metal-containing compound may include lanthanide metal telluride. Examples of the lanthanide metal telluride may be or include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and/or the like.

The alkali metal complex, the alkaline earth-metal complex, and the rare earth metal complex may include i) an ion of the alkali metal, the alkaline earth metal, or the rare earth metal, and ii) a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

In an embodiment, the electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, the compound represented by Formula 601).

In one or more embodiments, the electron injection layer may include (e.g., consist of): i) an alkali metal-containing compound (for example, an alkali metal halide); or ii) a) an alkali metal-containing compound (for example, an alkali metal halide), and b) an alkali metal, an alkaline earth metal, a rare earth metal, or any combination thereof. In one or more embodiments, the electron injection layer may be a KI:Yb co-deposited layer, an RbI:Yb co-deposited layer, and/or the like.

When the electron injection layer further includes an organic material, the alkali metal, alkaline earth metal, rare earth metal, alkali metal-containing compound, alkaline earth metal-containing compound, rare earth metal-containing compound, alkali metal complex, alkaline earth-metal complex, rare earth metal complex, or any combination thereof may be substantially homogeneously or non-homogeneously dispersed in a matrix including the organic material.

A thickness of the electron injection layer may be in a range of about 1 Å to about 100 Å, for example, about 3 Å to about 90 Å. When the thickness of the electron injection layer is within these ranges, satisfactory electron injection characteristics may be obtained without a substantial increase in driving voltage.

### Second Electrode 150

The second electrode 150 may be on the interlayer 130 having a structure as described above. The second electrode 150 may be a cathode, which is an electron injection electrode, and a material for forming the second electrode 150 may include a metal, an alloy, an electrically conductive compound, or any combination thereof, each having a low-work function.

The material for forming the second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

A first capping layer may be located outside the first electrode 110, and/or a second capping layer may be located outside the second electrode 150. In particular, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in the emission layer of the interlayer 130 of the light-emitting device 10 may be extracted toward the outside through the first electrode 110 (which is a semi-transmissive electrode or a transmissive electrode) and the first capping layer, and light generated in the emission layer of the interlayer 130 of the light-emitting device 10 may be extracted toward the outside through the second electrode 150 (which is a semi-transmissive electrode or a transmissive electrode) and the second capping layer.

The first capping layer and the second capping layer may increase external luminescence efficiency according to the principle of constructive interference. Accordingly, the light extraction efficiency of the light-emitting device 10 may be increased, so that the luminescence efficiency of the light-emitting device 10 may be improved.

Each of the first capping layer and the second capping layer may include a material having a refractive index of equal to or greater than 1.6 (at 589 nm).

The first capping layer and the second capping layer may each independently be an organic capping layer including an organic material, an inorganic capping layer including an inorganic material, or an organic-inorganic composite capping layer including an organic material and an inorganic material.

In an embodiment, at least one of the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may each optionally be substituted with a substituent including O, N, S, Se, Si, F, Cl, Br, I, or any combination thereof. In one or more embodiments, at least one of the first capping layer and the second capping layer may each independently include an amine group-containing compound.

For example, at least one of the first capping layer and the second capping layer may each independently include a compound represented by Formula 201, a compound represented by Formula 202, or any combination thereof.

In one or more embodiments, at least one of the first capping layer and the second capping layer may each independently include one of Compounds HT28 to HT33, one of Compounds CP1 to CP6, β-NPB, or any combination thereof:

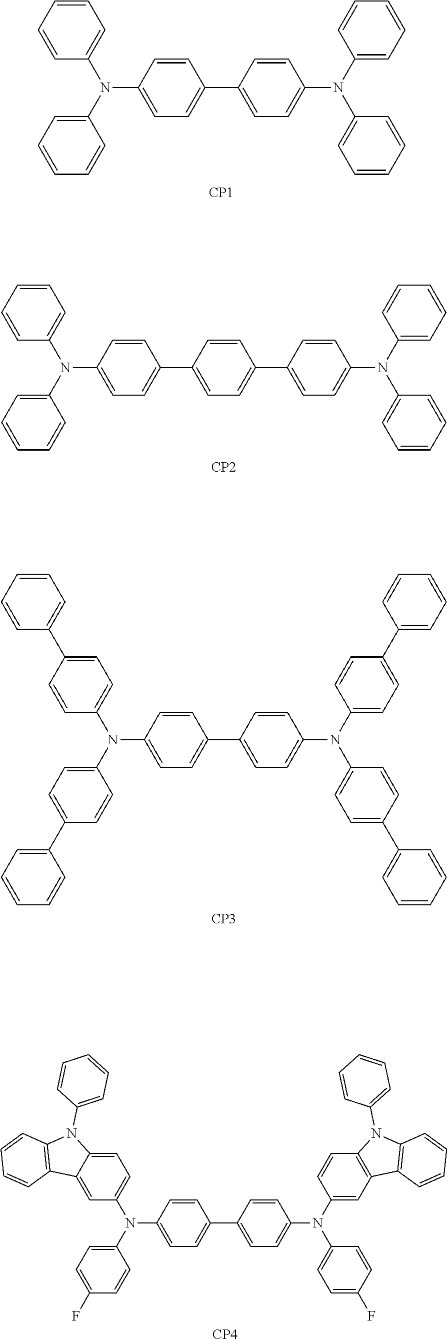

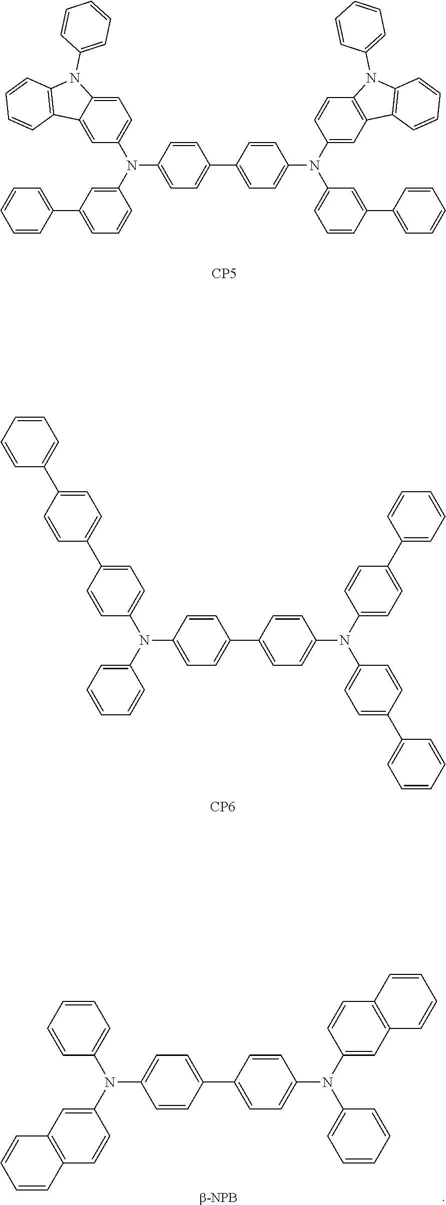 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; "CP5" ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; "CP6" ; c1ccc(N(c2ccc(-c3ccc(N(c4ccccc4)c4ccc5ccccc5c4)cc3)cc2)c2ccc3ccccc3c2)cc1]

### Electronic Apparatus

The light-emitting device may be included in one or more suitable electronic apparatuses. For example, the electronic apparatus including the light-emitting device may be a light-emitting apparatus, an authentication apparatus, and/or the like.

The electronic apparatus (for example, a light-emitting apparatus) may further include, in addition to the light-emitting device, i) a color filter, ii) a color conversion layer, or iii) both a color filter and a color conversion layer (e.g., simultaneously). The color filter and/or the color conversion layer may be arranged in at least one traveling direction of light emitted from the light-emitting device. For example, the light emitted from the light-emitting device may be blue light. For details on the light-emitting device, related description provided above may be referred to. In an embodiment, the color conversion layer may include quantum dots. The quantum dots may be, for example, the same as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be arranged among the subpixel areas to define each of the subpixel areas.

The color filter including the plurality of the color filter areas may further include light-shielding patterns interposed between the color filter areas, and the color conversion layer including the plurality of the color conversion areas may further include light-shielding patterns interposed between the color conversion areas.

The plurality of color filter areas (or the plurality of color conversion areas) may include a first area to emit first color light, a second area to emit second color light, and/or a third area to emit third color light, wherein the first color light, the second color light, and/or the third color light may have different maximum emission wavelengths from one another. For example, the first color light may be red light, the second color light may be green light, and the third color light may be blue light. For example, the color filter areas (and/or the color conversion areas) may include quantum dots. For example, the first area may include red quantum dots, the second area may include green quantum dots, and the third area may not include (e.g., may exclude) quantum dots. The quantum dots may be the same as described herein. Each of the first region, the second region, and/or the third region may further include a scatter.

In an embodiment, the light-emitting device may be to emit first light, the first area may be to absorb the first light to emit first first-color light, the second area may be to absorb the first light to emit second first-color light, and the third area may be to absorb the first light to emit third first-color light. In this regard, the first first-color light, the second first-color light, and the third first-color light may have different maximum emission wavelengths from one another. For example, the first light may be blue light, the first-first color light may be red light, the second-first color light may be green light, and the third-first color light may be blue light.

The electronic apparatus may further include a thin-film transistor, in addition to the light-emitting device described above. The thin-film transistor may include a source electrode, a drain electrode, and an activation layer, wherein any one of the source electrode and the drain electrode may be electrically connected to any one of the first electrode and the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

The electronic apparatus may further include a sealing portion for sealing the light-emitting device. The sealing portion and/or the color conversion layer may be arranged between the color filter and the light-emitting device. The sealing portion allows light from the light-emitting device to be extracted to the outside, while concurrently (e.g., simultaneously) preventing or reducing ambient air and moisture from penetrating into the light-emitting device. The sealing portion may be a sealing substrate including a transparent glass substrate and/or a plastic substrate. The sealing portion may be a thin-film encapsulation layer including at least one layer of an organic layer and/or an inorganic layer. When the sealing portion is a thin-film encapsulating layer, the electronic apparatus may be flexible.

Various functional layers may be additionally arranged on the sealing portion, in addition to the color filter and/or the color conversion layer, according to the intended use of the electronic apparatus. Examples of the functional layers may be or include a touch screen layer, a polarizing layer, and/or the like. The touch screen layer may be a pressure-sensitive touch screen layer, a capacitive touch screen layer, or an infrared touch screen layer. The authentication apparatus may be, for example, a biometric authentication apparatus that authenticates an individual by utilizing biometric information of a living body (for example, fingertips, pupils, and/or the like).

The authentication apparatus may further include, in addition to the light-emitting device as described above, a biometric information collector.

The electronic apparatus may be applied to one or more suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, various suitable measuring instruments, meters (for example, meters for a vehicle, an aircraft, and a vessel), projectors, and/or the like.

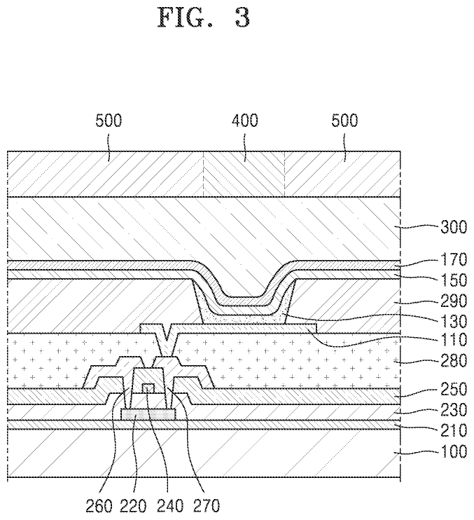

### Description of FIGS. 2 and 3

FIG. 2 is a cross-sectional view of an electronic apparatus according to an embodiment.

The electronic apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, and/or a metal substrate. A buffer layer 210 may be arranged on the substrate 100. The buffer layer 210 may prevent or reduce penetration of impurities through the substrate 100 and may provide a flat surface on the substrate 100.

The TFT may be arranged on the buffer layer 210. The TFT may include an activation layer 220, a gate electrode 240, a source electrode 260, and a drain electrode 270.

The activation layer 220 may include an inorganic semiconductor, (such as silicon or polysilicon), an organic semiconductor, or an oxide semiconductor, and may include a source region, a drain region, and a channel region.

A gate insulating film 230 for insulating the activation layer 220 from the gate electrode 240 may be arranged on the activation layer 220, and the gate electrode 240 may be arranged on the gate insulating film 230.

An interlayer insulating film 250 may be arranged on the gate electrode 240. The interlayer insulating film 250 may be arranged between the gate electrode 240 and the source electrode 260 and between the gate electrode 240 and the drain electrode 270, so as to provide insulation therebetween.

The source electrode 260 and the drain electrode 270 may be arranged on the interlayer insulating film 250. The interlayer insulating film 250 and the gate insulating film 230 may be formed to expose the source region and the drain region of the activation layer 220, and the source electrode 260 and the drain electrode 270 may be arranged to be in contact with the exposed portions of the source region and the drain region of the activation layer 220.

The TFT may be electrically connected to the light-emitting device to drive the light-emitting device, and may be covered by a passivation layer 280. The passivation layer 280 may include an inorganic insulating film, an organic insulating film, or any combination thereof. The light-emitting device may be provided on the passivation layer 280. The light-emitting device may include the first electrode 110, the interlayer 130, and the second electrode 150.

The first electrode 110 may be arranged on the passivation layer 280. The passivation layer 280 may expose a portion of the drain electrode 270 without completely covering the drain electrode 270, and the first electrode 110 may be arranged to be connected to the exposed portion of the drain electrode 270.

A pixel defining layer 290 including an insulating material may be arranged on the first electrode 110. The pixel defining layer 290 may expose a region of the first electrode 110, and the interlayer 130 may be formed in the exposed region of the first electrode 110. The pixel defining layer 290 may be a polyimide organic film or a polyacrylic organic film. In some embodiments, at least some layers of the interlayer 130 may extend beyond the upper portion of the pixel defining layer 290 to be arranged in the form of a common layer.

The second electrode 150 may be arranged on the interlayer 130, and a capping layer 170 may be additionally formed on the second electrode 150. The capping layer 170 may be formed to cover the second electrode 150.

The encapsulation portion 300 may be arranged on the capping layer 170. The encapsulation portion 300 may be arranged on the light-emitting device to protect the light-emitting device from moisture and/or oxygen. The encapsulation portion 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE) and/or the like), or any combination thereof; or any combination of the inorganic film and the organic film.

FIG. 3 is a cross-sectional view of an electronic apparatus according to another embodiment of the present disclosure.

The electronic apparatus of FIG. 3 is the same as the light-emitting apparatus of FIG. 2, except that a light-shielding pattern 500 and a functional region 400 are additionally located on the encapsulation portion 300. The functional region 400 may include i) a color filter area, ii) a color conversion area, or iii) a combination of a color filter area and a color conversion area. In one or more embodiments, the light-emitting device included in the light-emitting apparatus of FIG. 3 may be a tandem light-emitting device.

### Manufacturing Method

The respective layers included in the hole transport region, the emission layer, and the respective layers included in the electron transport region may be formed in a certain region by utilizing one or more suitable methods selected from vacuum deposition, spin coating, casting, Langmuir-Blodgett (LB) deposition, ink-jet printing, laser-printing, laser-induced thermal imaging, and/or the like.

When the respective layers included in the hole transport region, the emission layer, and the respective layers included in the electron transport region are formed by vacuum deposition, the deposition conditions may include a deposition temperature in a range of about 100° C. to about 500° C., a vacuum degree of about 10⁻⁸ torr to about 10⁻³ torr, and a deposition speed in a range of about 0.01 Å/sec to about 100 Å/sec, depending on a material to be included in a layer to be formed and the structure of a layer to be formed.

When the layers constituting the hole transport region, the emission layer, and the layers constituting the electron transport region are formed by spin coating, the spin coating may be performed at a coating speed of about 2,000 rpm to about 5,000 rpm and at a heat treatment temperature of about 80° C. to about 200° C. by taking into account a material to be included in a layer to be formed and the structure of a layer to be formed.

### DEFINITION OF TERMS

The term “C₃-C₆₀ carbocyclic group” as used herein refers to a cyclic group consisting of carbon only as a ring-forming atom and having three to sixty carbon atoms, and the term “C₁-C₆₀ heterocyclic group” as used herein refers to a cyclic group that has one to sixty carbon atoms and further has, in addition to carbon, a heteroatom as a ring-forming atom. Each of the C₃-C₆₀ carbocyclic group and the C₁-C₆₀ heterocyclic group may be a monocyclic group consisting of one ring or a polycyclic group consisting of two or more rings that are condensed together. For example, the C₁-C₆₀ heterocyclic group may have 3 to 61 ring-forming atoms.

The term “cyclic group” as used herein may include both (e.g., simultaneously) the C₃-C₆₀ carbocyclic group and the C₁-C₆₀ heterocyclic group.

The term “π electron-rich C₃-C₆₀ cyclic group” as used herein refers to a cyclic group that has 3 to 60 carbon atoms and does not include *—N═*′ as a ring-forming moiety, and the term “π electron-deficient nitrogen-containing C₁-C₆₀ cyclic group” as used herein refers to a heterocyclic group that has 1 to 60 carbon atoms and includes *—N=*′ as a ring-forming moiety.

For example,

the C₃-C₆₀ carbocyclic group may be i) a T1 group (defined below) or ii) a condensed cyclic group in which at least two T1 groups are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) a T2 group (defined below), ii) a condensed cyclic group in which at least two T2 groups are condensed with each other, or iii) a condensed cyclic group in which at least one T2 group and at least one T1 group are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),

the π electron-rich C₃-C₆₀ cyclic group may be i) a T1 group, ii) a condensed cyclic group in which at least two T1 groups are condensed with each other, iii) a T3 group (defined below), iv) a condensed cyclic group in which at least two T3 groups are condensed with each other, or v) a condensed cyclic group in which at least one T3 group and at least one T1 group are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like),

the π electron-deficient nitrogen-containing C₁-C₆₀ cyclic group may be i) a T4 group (defined below), ii) a condensed cyclic group in which at least two T4 groups are condensed with each other, iii) a condensed cyclic group in which at least one T4 group and at least one T1 group are condensed with each other, iv) a condensed cyclic group in which at least one T4 group and at least one T3 group are condensed with each other, or v) a condensed cyclic group in which at least one T4 group, at least one T1 group, and at least one T3 group are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),

the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,

the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,

the T3 group may be a furan group, a thiophene group, a 1H-pyrrole group, a silole group, or a borole group, and

the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group, the C₃-C₆₀ carbocyclic group, the C₁-C₆₀ heterocyclic group, the π electron-rich C₃-C₆₀ cyclic group, or the π electron-deficient nitrogen-containing C₁-C₆₀ cyclic group” as used herein refer to a group condensed to any cyclic group, a monovalent group, or a polyvalent group (for example, a divalent group, a trivalent group, a tetravalent group, and/or the like) according to the structure of a formula for which the corresponding term is used. For example, the “benzene group” may be a benzo group, a phenyl group, a phenylene group, and/or the like, which may be easily understood by one of ordinary skill in the art according to the structure of a formula including the “benzene group”.

Examples of the monovalent C₃-C₆₀ carbocyclic group and the monovalent C₁-C₆₀ heterocyclic group may include a C₃-C₁₀ cycloalkyl group, a C₁-C₆₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and/or a monovalent non-aromatic condensed heteropolycyclic group. Examples of the divalent C₃-C₆₀ carbocyclic group and the divalent C₁-C₆₀ heterocyclic group may include a C₃-C₁₀ cycloalkylene group, a C₁-C₆₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and/or a substituted or unsubstituted divalent non-aromatic condensed heteropolycyclic group.

The term “C₁-C₆₀ alkyl group” as used herein refers to a linear or branched aliphatic hydrocarbon monovalent group that has one to sixty carbon atoms, and examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an iso-nonyl group, a sec-nonyl group, a tert-nonyl group, an n-decyl group, an isodecyl group, a sec-decyl group, a tert-decyl group, and/or the like. The term “C₁-C₆₀ alkylene group” as used herein refers to a divalent group having substantially the same structure as the C₁-C₆₀ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and examples thereof may include an ethenyl group, a propenyl group, a butenyl group, and/or the like. The term “C₂-C₆₀ alkenylene group” as used herein refers to a divalent group having substantially the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and examples thereof may include an ethynyl group, a propynyl group, and/or the like. The term “C₂-C₆₀ alkynylene group” as used herein refers to a divalent group having substantially the same structure as the C₂-C₆₀ alkynyl group.

The term “C₁-C₆₀ alkoxy group” as used herein refers to a monovalent group represented by —OA₁₀₁ (wherein A₁₀₁ is a C₁-C₆₀ alkyl group), and examples thereof may include a methoxy group, an ethoxy group, an isopropyloxy group, and/or the like.

The term “C₃-C₁₀ cycloalkyl group” as used herein refers to a monovalent saturated hydrocarbon cyclic group having three to ten carbon atoms, and examples thereof may include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, a bicyclo[2.2.2]octyl group, and/or the like. The term “C₃-C₁₀ cycloalkylene group” as used herein refers to a divalent group having substantially the same structure as the C₃-C₁₀ cycloalkyl group.

The term “C₁-C₁₀ heterocycloalkyl group” as used herein refers to a monovalent cyclic group that further includes, in addition to a carbon atom, at least one heteroatom as a ring-forming atom and has 1 to 10 carbon atoms, and examples thereof may include a 1,2,3,4-oxatriazolidinyl group, a tetrahydrofuranyl group, a tetrahydrothiophenyl group, and/or the like. The term “C₁-C₁₀ heterocycloalkylene group” as used herein refers to a divalent group having substantially the same structure as the C₁-C₁₀ heterocycloalkyl group.

The term “C₃-C₁₀ cycloalkenyl group” as used herein refers to a monovalent cyclic group that has 3 to 10 carbon atoms, at least one carbon-carbon double bond in the ring thereof, and no aromaticity, and examples thereof may include a cyclopentenyl group, a cyclohexenyl group, a cycloheptenyl group, and/or the like. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent cyclic group that has, in addition to a carbon atom, at least one heteroatom as a ring-forming atom, 1 to 10 carbon atoms, and at least one carbon-carbon double bond in the cyclic structure thereof. Examples of the C₁-C₁₀ heterocycloalkenyl group may include a 4,5-dihydro-1,2,3,4-oxatriazolyl group, a 2,3-dihydrofuranyl group, a 2,3-dihydrothiophenyl group, and/or the like. The term “C₁-C₁₀ heterocycloalkenylene group” as used herein refers to a divalent group having substantially the same structure as the C₁-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and/or the like. When the C₆-C₆₀ aryl group and the C₆-C₆₀ arylene group each include two or more rings, the rings may be condensed with each other.

The term “C₁-C₆₀ heteroaryl group” as used herein refers to a monovalent group having a heterocyclic aromatic system that has, in addition to a carbon atom, at least one heteroatom as a ring-forming atom, and 1 to 60 carbon atoms. The term “C₁-C₆₀ heteroarylene group” as used herein refers to a divalent group having a heterocyclic aromatic system that has, in addition to a carbon atom, at least one heteroatom as a ring-forming atom, and 1 to 60 carbon atoms. Examples of the C₁-C₆₀ heteroaryl group may include a pyridinyl group, a pyrimidinyl group, a pyrazinyl group, a pyridazinyl group, a triazinyl group, a quinolinyl group, a benzoquinolinol group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and/or the like. When the C₁-C₆₀ heteroaryl group and the C₁-C₆₀ heteroarylene group each include two or more rings, the rings may be condensed with each other.

The term “monovalent non-aromatic condensed polycyclic group” as used herein refers to a monovalent group (for example, having 8 to 60 carbon atoms) having two or more rings condensed to each other, only carbon atoms as ring-forming atoms, where the entire molecular structure is not aromatic (e.g., an aromatic conjugation system does not extend throughout the entire structure). Examples of the monovalent non-aromatic condensed polycyclic group may include an indenyl group, a fluorenyl group, a spiro-bifluorenyl group, a benzofluorenyl group, an indenophenanthrenyl group, and/or an indeno anthracenyl group. The term “divalent non-aromatic condensed polycyclic group” as used herein refers to a divalent group having substantially the same structure as the monovalent non-aromatic condensed polycyclic group described above.

The term “monovalent non-aromatic condensed heteropolycyclic group” as used herein refers to a monovalent group (for example, having 1 to 60 carbon atoms) having two or more rings condensed with each other, at least one heteroatom other than carbon atoms, as a ring-forming atom, where the entire molecular structure is not aromatic (e.g., an aromatic conjugation system does not extend throughout the entire structure). Examples of the monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, and/or the like. The term “divalent non-aromatic condensed heteropolycyclic group” as used herein refers to a divalent group having the same structure as the monovalent non-aromatic condensed heteropolycyclic group described above.

The term “C₆-C₆₀ aryloxy group” as used herein indicates —OA₁₀₂ (wherein A₁₀₂ is the C₆-C₆₀ aryl group), and the term “C₆-C₆₀ arylthio group” as used herein indicates —SA₁₀₃ (wherein A₁₀₃ is a C₆-C₆₀ aryl group).

The term “C₇-C₆₀ arylalkyl group” as used herein refers to -A₁₀₄A₁₀₅ (wherein A₁₀₄ is a C₁-C₅₄ alkylene group and A₁₀₅ is a C₆-C₅₉ aryl group), and the term “C₂-C₆₀ heteroarylalkyl group” as used herein refers to -A₁₀₆A107 (wherein A₁₀₆ is a C₁-C₅₉ alkylene group and A₁₀₇ is a C₁-C₆₉ heteroaryl group).

The term “R₁₀ₐ” as used herein may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and

Q₁ to Q₃, Q₁₁ to Q₁₃, Q₂₁ to Q₂₃ and Q₃₁ to Q₃₃ used herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ aryl alkyl group; or a C₂-C₆₀ heteroaryl alkyl group.

The term “heteroatom” as used herein refers to any atom other than a carbon atom. Examples of the heteroatom may include O, S, N, P, Si, B, Ge, Se, or any combination thereof.

The term “the third-row transition metal” used herein includes hafnium (Hf), tantalum (Ta), tungsten (W), rhenium (Re), osmium (Os), iridium (Ir), platinum (Pt), gold (Au), and/or the like.

“Ph” as used herein refers to a phenyl group, “Me” as used herein refers to a methyl group, “Et” as used herein refers to an ethyl group, “ter-Bu” or “But” as used herein refers to a tert-butyl group, and “OMe” as used herein refers to a methoxy group.

The term “biphenyl group” as used herein refers to “a phenyl group substituted with a phenyl group”. In other words, the “biphenyl group” belongs to “a substituted phenyl group having a C₆-C₆₀ aryl group as a substituent”.

The term “terphenyl group” as used herein refers to “a phenyl group substituted with a biphenyl group”. In other words, the “terphenyl group” belongs to “a substituted phenyl group having, as a substituent, a C₆-C₆₀ aryl group substituted with a C₆-C₆₀ aryl group”.

* and *′ as used herein, unless defined otherwise, each refer to a binding site to a neighboring atom in a corresponding formula.

Hereinafter, a compound and light-emitting device according to embodiments will be described in more detail with reference to Examples.

### EXAMPLES

### Evaluation Example

### Evaluation of Hole Mobility, Electron Mobility, HOMO Energy, and LUMO Energy

By driving a hole-only device and an electron-only device and characterizing the devices by utilizing a source meter (2400 series, Keithley Instruments Inc.) and a high impedance electrometer (Keithley 6514), the hole mobility and the electron mobility of the compounds of Table 1 were measured at room temperature (25° C.). The measurement results and HOMO and LUMO energy values of the compounds are shown in Table 1.

The structures of the hole-only device and the electron-only device utilized for measurements of the hole mobility and the electron mobility are as follows:

### Hole-Only Device

ITO (1,500 Å)/HT3:NDP9 (1 wt %) (100 Å)/HT3 (500 Å)/compounds of Table 1 (1,000 Å)/NDP-9 (100 Å)/Yb (13 Å)/Ag:Mg (10 wt %) (600 Å); and

### Electron-Only Device

ITO (1,500 Å)/Ag:Mg (300 Å)/Yb(13 Å)/TPM-TAZ:LiQ (50 wt %) (300 Å) /compounds of Table 1 (1,000 Å)/TPM-TAZ:LiQ (50 wt %) (300 Å)/Yb (13 Å)/Ag:Mg (10 wt %) (600 Å).

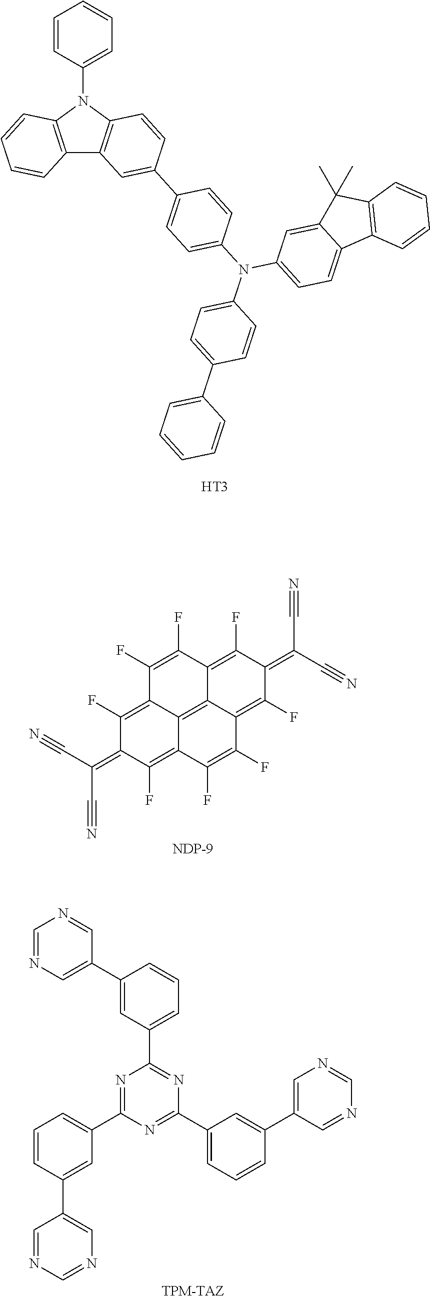

TABLE 1

| | Hole | Electron | HOMO | LUMO |
|---|---|---|---|---|
| | mobility | mobility | energy | energy |
| Compound | (V · s/cm²) | (V · s/cm²) | (eV) | (eV) |
| 1-6 | 3.5 × 10⁻³ | 1.9 × 10⁻⁶ | −5.40 | −1.90 |
| 1-9 | 5.3 × 10⁻⁴ | 3.7 × 10⁻⁶ | −5.38 | −1.88 |
| 2-19 | 3.3 × 10⁻⁶ | 2.3 × 10⁻⁴ | −5.68 | −2.86 |
| 2-20 | 8.1 × 10⁻⁶ | 1.0 × 10⁻⁵ | −5.97 | −2.63 |

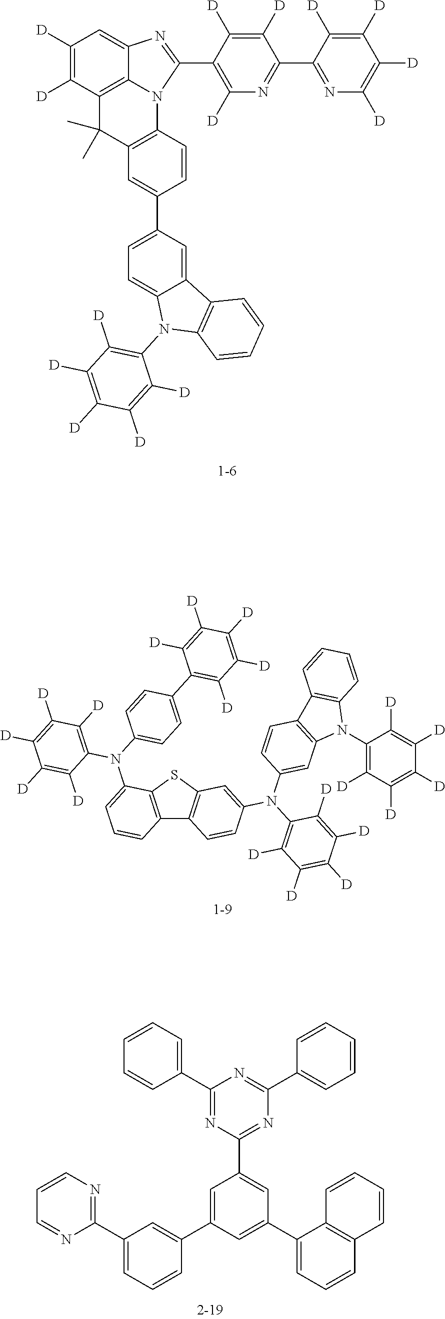 [SMILES: [2H]c1cc2nc(-c3c([2H])nc(-c4nc([2H])c([2H])c([2H])c4[2H])c([2H])c3[2H])n3c2c(c1[2H])C(C)(C)c1cc(-c2ccc4c(c2)c2ccccc2n4-c2c([2H])c([2H])c([2H])c([2H])c2[2H])ccc1-3 ; [2H]c1c([2H])c([2H])c(-c2ccc(N(c3c([2H])c([2H])c([2H])c([2H])c3[2H])c3cccc4c3sc3cc(N(c5ccc6c7ccccc7n(-c7c([2H])c([2H])c([2H])c([2H])c7[2H])c6c5)c5c([2H])c([2H])c([2H])c([2H])c5[2H])ccc34)cc2)c([2H])c1[2H] ; c1ccc(-c2nc(-c3ccccc3)nc(-c3cc(-c4cccc(-c5ncccn5)c4)cc(-c4cccc5ccccc45)c3)n2)cc1]

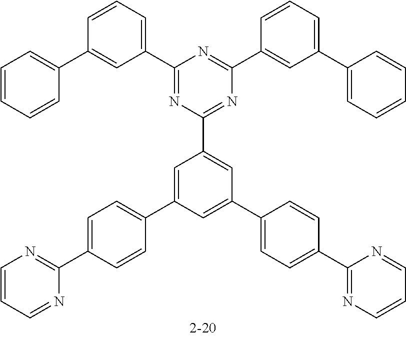 [SMILES: c1ccc(-c2cccc(-c3nc(-c4cccc(-c5ccccc5)c4)nc(-c4cc(-c5ccc(-c6ncccn6)cc5)cc(-c5ccc(-c6ncccn6)cc5)c4)n3)c2)cc1]

Manufacture of Light-Emitting Device

### Comparative Example 1

An ITO glass substrate was cut to a size of 50 mm×50 mm×0.7 mm, ultrasonically cleaned with isopropyl alcohol and pure water each for 10 minutes, and then cleaned by irradiation of ultraviolet rays and exposure to ozone for 10 minutes. Then, the ITO glass substrate was loaded onto a vacuum deposition apparatus. HAT-CN was vacuum-deposited on the ITO glass substrate to form a hole injection layer having a thickness of 50 Å, and then NPB was vacuum-deposited on the hole injection layer to form a hole transport layer having a thickness of 600 Å and TCTA was vacuum-deposited on the hole transport layer to form a hole injection layer having a thickness of 50 Å.

Compounds 1-6′ and 2-19 as hosts (at a weight ratio of 5:5) and Compound G-1 as a dopant were co-deposited on the hole injection layer to form a single-layered emission layer having a thickness of 200 Å (at a doping concentration of 5 wt %).

Subsequently, T2T was vacuum-deposited on the single-layered emission layer to form a hole blocking layer having a thickness of 50 Å. TPM-TAZ was vacuum-deposited on the hole blocking layer to form an electron transport layer having a thickness of 300 Å.

Ag:Mg (10 wt %) were vacuum-deposited on the electron transport layer to form an electrode having a thickness of 100 Å, and CPL was vacuum-deposited on the Ag:Mg (10 wt %) electrode to form a capping layer having a thickness of 700 Å, thereby completing manufacture of a light-emitting device.

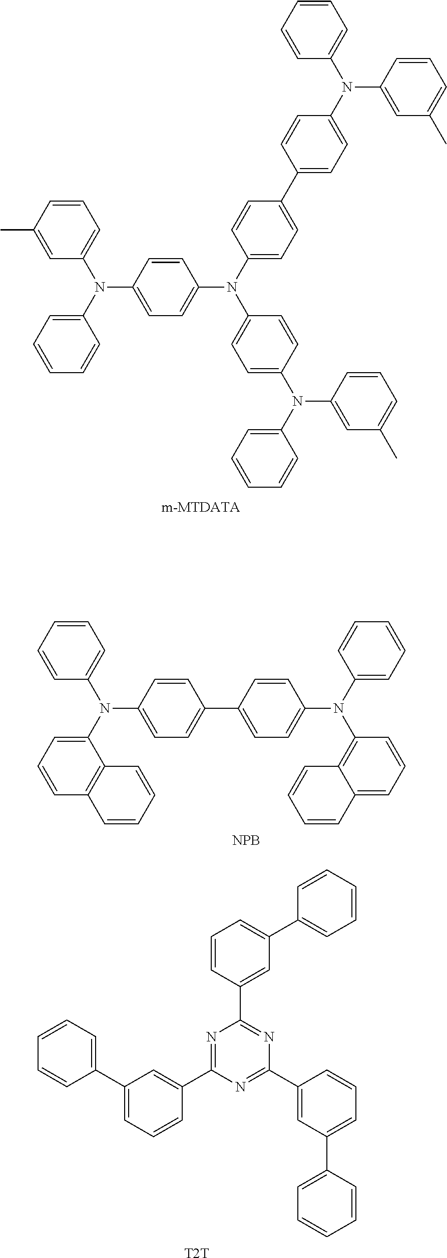

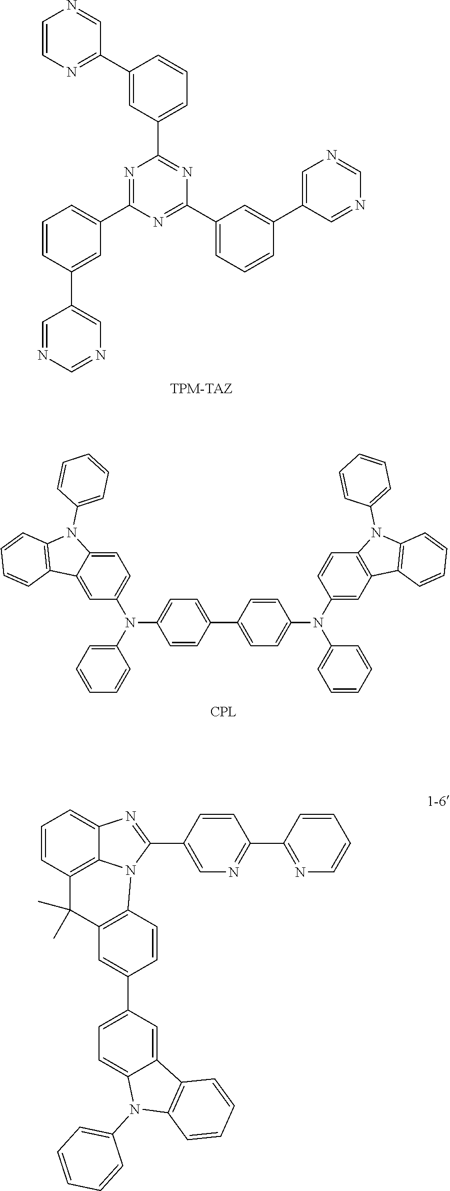 [SMILES: c1cc(-c2cncnc2)cc(-c2nc(-c3cccc(-c4cncnc4)c3)nc(-c3cccc(-c4cnccn4)c3)n2)c1 ; "TPM-TAZ" ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2cc(-c3ccc4c(c3)c3ccccc3n4-c3ccccc3)ccc2-n2c(-c3ccc(-c4ccccn4)nc3)nc3cccc1c32]

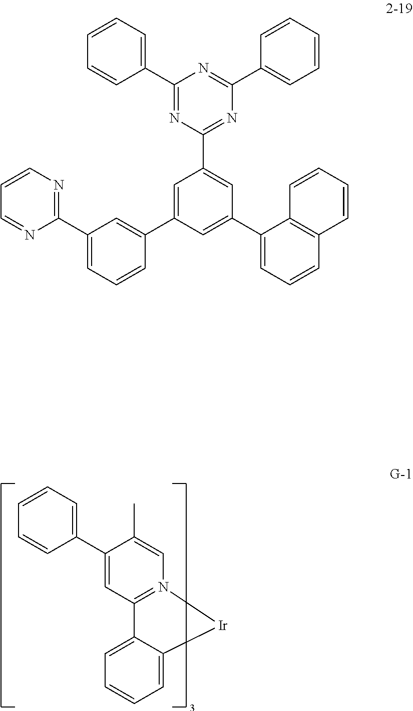

### Comparative Example 2

A light-emitting device was manufactured in substantially the same manner as in Comparative Example 1, except that, in forming an emission layer, Compounds 1-9′ and 2-20 were utilized as hosts (at a weight ratio of 5:5) instead of Compounds 1-6′ and 2-19, and Compound G-7 was utilized as a dopant instead of Compound G-1, and that the hosts and the dopant that were newly utilized herein were co-deposited to form a single-layered emission layer having a thickness of 400 Å (at a doping concentration of 5 wt %).

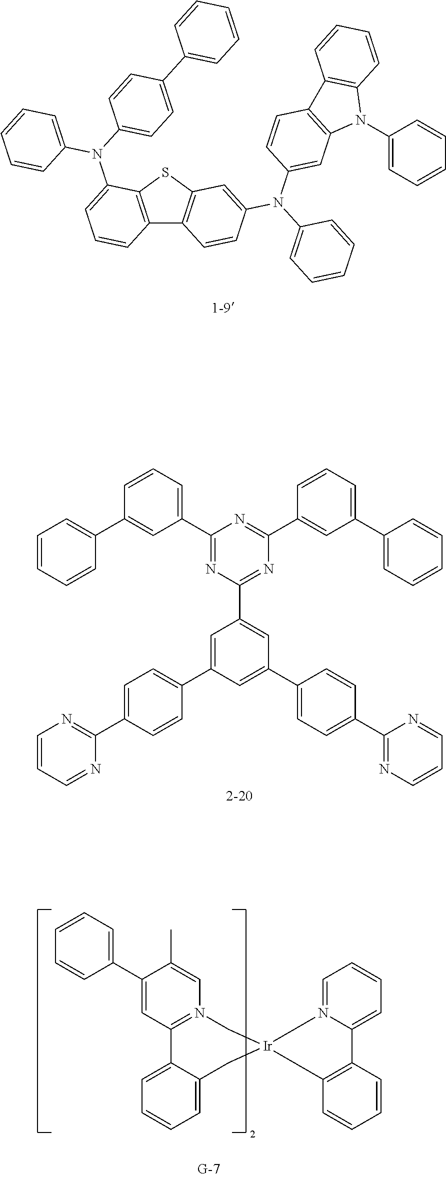

### Comparative Example 3

A light-emitting device was manufactured in substantially the same manner as in Comparative Example 1, except that, in forming an emission layer, Compound G-7 was utilized as a dopant instead of Compound G-1, and that the hosts of Comparative Example 1 and the dopant newly utilized herein were co-deposited to form a single-layered emission layer having a thickness of 400 Å (at a doping concentration of 5 wt %).

### Comparative Example 4

A light-emitting device was manufactured in substantially the same manner as in Comparative Example 2, except that, in forming an emission layer, Compound G-7 was utilized as a dopant instead of Compound G-1, and that the hosts of Comparative Example 2 and the dopant newly utilized herein were co-deposited to form a single-layered emission layer having a thickness of 400 Å (at a doping concentration of 5 wt %).

### Comparative Example 5

A light-emitting device was manufactured in substantially the same manner as in Comparative Example 1, except that, in forming an emission layer, Compounds 1-6′ and 2-19 were utilized as hosts (at a weight ratio of 5:5) and Compound G-1 was utilized as a dopant, and the hosts and the dopant that were newly utilized herein were co-deposited to form a first emission layer having a thickness of 100 Å (at a doping concentration of 5 wt %), and that Compounds 1-9′ and 2-20 were utilized as hosts (at a weight ratio of 5:5) and Compound G-7 was utilized as a dopant, and the hosts and the dopant that were newly utilized herein were co-deposited on the first emission layer to form a second emission layer having a thickness of 100 Å (at a doping concentration of 5 wt %).

### Comparative Example 6

A light-emitting device was manufactured in substantially the same manner as in Comparative Example 5, except that, in forming an emission layer, Compounds 1-9′ and 2-20 were utilized as hosts (at a weight ratio of 5:5) and Compound G-7 was utilized as a dopant, and the hosts and the dopant that were newly utilized herein were co-deposited to form a first emission layer having a thickness of 100 Å (at a doping concentration of 5 wt %), and that Compounds 1-6′ and 2-19 were utilized as hosts (at a weight ratio of 5:5) and Compound G-1 was utilized as a dopant, and the hosts and the dopant that were newly utilized herein were co-deposited on the first emission layer to form a second emission layer having a thickness of 100 Å (at a doping concentration of 5 wt %).

### Comparative Example 7

A light-emitting device was manufactured in substantially the same manner as in Comparative Example 5, except that Compound G-7 was utilized as a dopant instead of Compound G-1 in forming a first emission layer, and that Compound G-1 was utilized as a dopant instead of Compound G-7 in forming a second emission layer.

### Comparative Example 8

A light-emitting device was manufactured in substantially the same manner as in Comparative Example 6, except that Compound G-1 was utilized as a dopant instead of Compound G-7 in forming a first emission layer, and that Compound G-7 was utilized as a dopant instead of Compound G-1 in forming a second emission layer.

### Comparative Example 9

A light-emitting device was manufactured in substantially the same manner as in Comparative Example 5, except that Compounds 1-6′ and 2-19 were utilized as hosts at a weight ratio of 3:7 in forming a first emission layer.

### Comparative Example 10

A light-emitting device was manufactured in substantially the same manner as in Comparative Example 5, except that Compounds 1-6′ and 2-19 were utilized as hosts at a weight ratio of 7:3 in forming a first emission layer.

### Comparative Example 11

A light-emitting device was manufactured in substantially the same manner as in Comparative Example 5, except that Compounds 1-9′ and 2-20 were utilized as hosts at a weight ratio of 7:3 in forming a second emission layer.

### Comparative Example 12

A light-emitting device was manufactured in substantially the same manner as in Comparative Example 5, except that Compounds 1-9′ and 2-20 were utilized as hosts at a weight ratio of 3:7 in forming a second emission layer.

To evaluate the characteristics of the light-emitting devices of Comparative Examples 1 to 12, the driving voltage at a current density of 10 mA/cm², efficiency, and lifespan were measured, and the results are shown in Table 2.

The driving voltage and the current density of the light-emitting devices were measured utilizing a source meter (2400 series, Keithley Instruments Inc.), and the efficiency of the light-emitting devices was measured utilizing a measurement system (C9920-2-12 of Hamamatsu Photonics Inc.).

TABLE 2

| | | | Driving | | | | |
|---|---|---|---|---|---|---|---|
| | | | voltage | | Efficiency | Efficiency | Lifespan |
| | | | @ 100 | Efficiency | @ 1000 | @ 10000 | @ 10000 |
| | | | nit | @ 100 nit | nit | nit | nit |
| | [Host] & {Dopant} | | (V) | (Cd/A) | (Cd/A) | (Cd/A) | (T95) |
| | Single-layered EML | | | | | | |
| Comparative | [1-6′, 2-19] & {G-1} | | 100% | 100% | 89% | 67% | 300 h |
| Example 1 | | | | | | | |
| Comparative | [1-9′, 2-20] & {G-7} | | 100% | 100% | 90% | 71% | 300 h |
| Example 2 | | | | | | | |
| Comparative | [1-6′, 2-19] & {G-7} | | 104% | 94% | 85% | 65% | 260 h |
| Example 3 | | | | | | | |
| Comparative | [1-9′, 2-20] & {G-1} | | 105% | 92% | 83% | 62% | 250 h |
| Example 4 | | | | | | | |
| | EML1 | EML2 | | | | | |
| Comparative | [1-6′, 2-19] | [1-9′, 2- | 100% | 100% | 98% | 94% | 380 h |
| Example 5 | & {G-1} | 20] & {G- | | | | | |
| | | 7) | | | | | |
| Comparative | [1-9′, 2-20] | [1-6′, 2- | 105% | 100% | 89% | 68% | 250 h |
| Example 6 | & {G-7} | 19] & {G- | | | | | |
| | | 1} | | | | | |
| Comparative | [1-6′, 2-19] | [1-9′, 2- | 104% | 93% | 91% | 86% | 350 h |
| Example 7 | & {G-7} | 20] & {G- | | | | | |
| | | 1} | | | | | |
| Comparative | [1-9′, 2-20] | [1-6′, 2- | 105% | 93% | 82% | 61% | 150 h |
| Example 8 | & {G-1} | 19] & {G- | | | | | |
| | | 7) | | | | | |
| Comparative | [1-6′, 2-19] | [1-9′, 2- | 100% | 102% | 98% | 96% | 370 h |
| Example 9 | & {G-1} | 20] & {G- | | | | | |
| | (1-6′:2- | 7} | | | | | |
| | 19 = 3:7)¹⁾ | (1-9′:2- | | | | | |
| | | 20 = 5:5) | | | | | |
| Comparative | [1-6′, 2-19] | [1-9′, 2- | 100% | 99% | 96% | 93% | 380 h |
| Example 10 | & {G-1} | 20] & {G- | | | | | |
| | (1-6′:2- | 7} | | | | | |
| | 19 = 7:3) | (1-9′:2- | | | | | |
| | | 20 = 5:5) | | | | | |
| Comparative | [1-6′, 2-19] | [1-9′, 2- | 100% | 100% | 97% | 95% | 400 h |
| Example 11 | & {G-1] | 20] & {G- | | | | | |
| | (1-6′:2- | 7} | | | | | |
| | 19 = 5:5) | (1-9′:2- | | | | | |
| | | 20 = 7:3) | | | | | |
| Comparative | [1-6′, 2-19] | [1-9′, 2- | 101% | 98% | 96% | 93% | 410 h |
| Example 12 | & {G-1] | 20] & {G- | | | | | |
| | (1-6′:2- | 7} | | | | | |
| | 19 = 5:5) | (1-9′:2- | | | | | |
| | | 20 = 3:7) | | | | | |

In Table 2, ratios in parentheses indicate weight ratios between hosts.

### Comparative Examples 13 to 18 and Examples 1 to 6

Additional light-emitting devices were respectively manufactured in substantially the same manner as described in Table 2, except that, in forming an emission layer, Compound 1-6 was utilized instead of Compound 1-6′ and Compound 1-9 was utilized instead of Compound 1-9′.

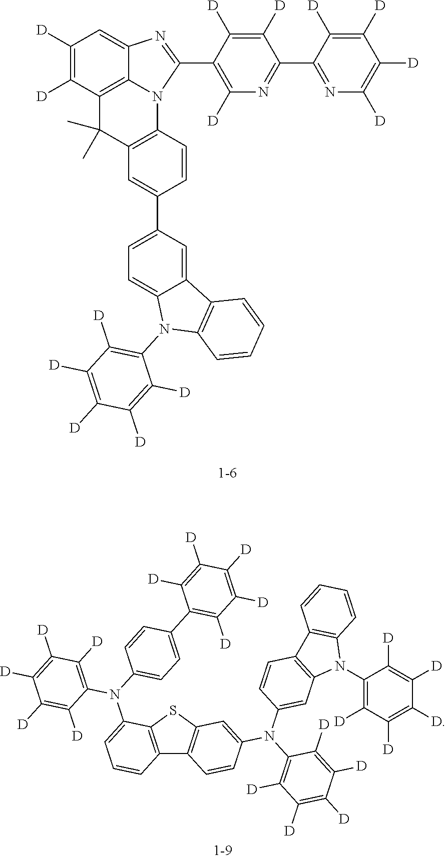

The results are shown in Table 3.

TABLE 3

| | | | Driving | | | | |
|---|---|---|---|---|---|---|---|
| | | | voltage | | Efficiency | Efficiency | Lifespan |
| | | | @ 100 | Efficiency | @ 1000 | @ 10000 | @ 10000 |
| | | | nit | @ 100 nit | nit | nit | nit |
| | [Host] & {Dopant} | | (V) | (Cd/A) | (Cd/A) | (Cd/A) | (T95) |
| | Single-layered EML | | | | | | |
| Comparative | [1-6, 2-19] & {G-1} | | 100% | 100% | 89% | 68% | 320 h |
| Example 13 | | | | | | | |
| Comparative | [1-9, 2-20] & {G-7] | | 100% | 100% | 90% | 70% | 330 h |
| Example 14 | | | | | | | |
| Comparative | [1-6, 2-19] & {G-7] | | 104% | 94% | 85% | 66% | 290 h |
| Example 15 | | | | | | | |
| Comparative | [1-9, 2-20] & {G-1} | | 105% | 92% | 83% | 64% | 270 h |
| Example 16 | | | | | | | |
| | EML1 | EML2 | | | | | |
| Example 1 | [1-6, 2-19] | [1-9, 2- | 100% | 100% | 98% | 96% | 730 h |
| | & {G-1} | 20] & {G- | | | | | |
| | | 7) | | | | | |
| Comparative | [1-9, 2-20] | [1-6, 2- | 105% | 100% | 89% | 69% | 250 h |
| Example 17 | & {G-7} | 19] & {G- | | | | | |
| | | 1} | | | | | |
| Example 2 | [1-6, 2-19] | [1-9, 2- | 104% | 93% | 91% | 88% | 640 h |
| | & {G-7} | 20] & {G- | | | | | |
| | | 1} | | | | | |
| Comparative | [1-9, 2-20] | [1-6, 2- | 105% | 93% | 82% | 62% | 150 h |
| Example 18 | & {G-1] | 19] & {G- | | | | | |
| | | 7) | | | | | |
| Example 3 | [1-6, 2-19] | [1-9, 2- | 100% | 102% | 98% | 95% | 720 h |
| | & {G-1] | 20] & {G- | | | | | |
| | (1-6:2- | 7} | | | | | |
| | 19 = 3:7)¹⁾ | (1-9:2- | | | | | |
| | | 20 = 5:5) | | | | | |
| Example 4 | [1-6, 2-19] | [1-9, 2- | 100% | 99% | 95% | 93% | 730 h |
| | & {G-1] | 20] & {G- | | | | | |
| | (1-6:2- | 7} | | | | | |
| | 19 = 7:3) | (1-9:2- | | | | | |
| | | 20 = 5:5) | | | | | |
| Example 5 | [1-6, 2-19] | [1-9, 2- | 100% | 100% | 97% | 95% | 720 h |
| | & {G-1] | 20] & {G- | | | | | |
| | (1-6:2- | 7} | | | | | |
| | 19 = 5:5) | (1-9:2- | | | | | |
| | | 20 = 7:3) | | | | | |
| Example 6 | [1-6, 2-19] | [1-9, 2- | 101% | 98% | 95% | 93% | 710 h |
| | & {G-1] | 20] & {G- | | | | | |
| | | 7) | | | | | |
| | (1-6:2- | (1-9:2- | | | | | |
| | 19 = 5:5) | 20 = 3:7) | | | | | |

In Table 3, ratios in a parentheses indicate weight ratios between hosts.

The light-emitting devices of Comparative Examples 13 to 16 each included a deuterium-substituted hole-transporting host, but had a single-layered emission layer. The light-emitting devices of Comparative Examples 17 and 18 had HOMO and LUMO energy relationships opposite that of the light-emitting devices of Examples 1 and 2, respectively (e.g., the compositions of EML1 and EML2 were switched or exchanged when Comparative Example 17 is compared with Example 1, and Comparative Example 18 is compared with Example 2).

It was confirmed that the light-emitting devices of the Examples exhibited excellent or suitable efficiency and/or long lifespan characteristics, as compared to the light-emitting devices of the Comparative Examples.

All of the light-emitting devices of the Comparative Examples and the Examples showed a tendency to decrease in efficiency (e.g., due to the roll-off phenomenon) as the luminance increased.

However, all the light-emitting devices of the Examples showed less reduction in efficiency upon the roll-off phenomenon, as compared to the light-emitting devices of Comparative Examples.

Comparing the results of Table 3 with the results of Table 2, it was confirmed that the light-emitting devices of Examples 1 to 6, each utilizing a deuterium-substituted hole-transporting host, had longer lifespans than the light-emitting devices of Comparative Examples 5, 6, and 9 to 12, each utilizing a deuterium-free hole-transporting host.

### Comparative Examples 19 to 30

Light-emitting devices were manufactured in substantially the same manner as described in Table 2, except that, in forming an emission layer, Compound 2-19′ including deuterium was utilized instead of Compound 2-19 and Compound 2-20′ including deuterium was utilized instead of Compound 2-20.

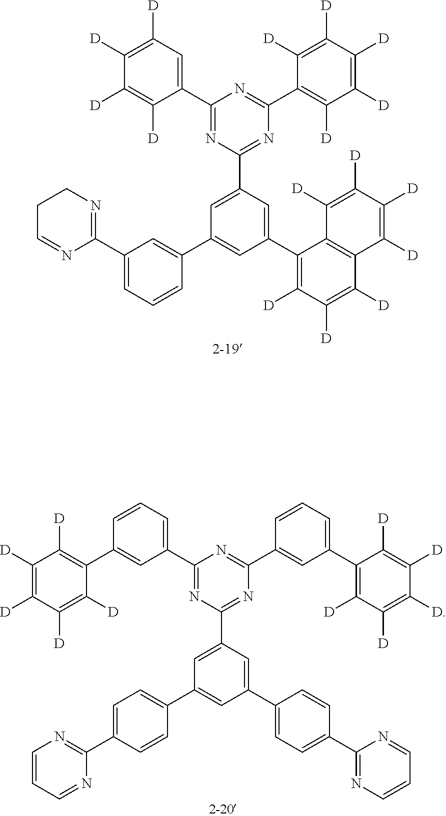

The results are shown in Table 4.

TABLE 4

| | | | Driving | | | | |
|---|---|---|---|---|---|---|---|
| | | | voltage | | | | |
| | | | @ 100 | Efficiency | Efficiency | Efficiency | Lifespan |
| | | | nit | @ 100 nit | @ 1000 nit | @ 10000 nit | @ 10000 nit |
| | [Host] & {Dopant} | | (V) | (Cd/A) | (Cd/A) | (Cd/A) | (T95) |
| | Single-layered EML | | | | | | |
| Comparative | [1-6, 2-19′] & {G-1} | | 100% | 100% | 88% | 66% | 290 h |
| Example 19 | | | | | | | |
| Comparative | [1-9, 2-20′] & {G-7] | | 100% | 100% | 88% | 70% | 305 h |
| Example 20 | | | | | | | |
| Comparative | [1-6, 2-19′] & {G-7] | | 104% | 95% | 83% | 64% | 264 h |
| Example 21 | | | | | | | |
| Comparative | [1-9, 2-20′] & {G-1} | | 105% | 93% | 82% | 63% | 252 h |
| Example 22 | | | | | | | |
| | EML1 | EML2 | | | | | |
| Comparative | [1-6, 2- | [1-9, 2- | 100% | 100% | 98% | 93% | 385 h |
| Example 23 | 19′] & | 20′] & | | | | | |
| | (G-1) | {G-7} | | | | | |
| Comparative | [1-9, 2- | [1-6, 2- | 105% | 100% | 88% | 67% | 255 h |
| Example 24 | 20′] & | 19′] & | | | | | |
| | (G-7) | {G-1} | | | | | |
| Comparative | [1-6, 2- | [1-9, 2- | 104% | 94% | 92% | 87% | 351 h |
| Example 25 | 19′] & | 20′] & | | | | | |
| | {G-7} | {G-1} | | | | | |
| Comparative | [1-9, 2- | [1-6, 2- | 105% | 93% | 82% | 61% | 154 h |
| Example 26 | 20′] & | 19′] & | | | | | |
| | {G-1} | {G-7} | | | | | |
| Comparative | [1-6, 2- | [1-9, 2- | 100% | 101% | 97% | 95% | 374 h |
| Example 27 | 19′] & | 20′] & | | | | | |
| | {G-1} | {G-7} | | | | | |
| | (1-6:2- | (1-9:2- | | | | | |
| | 19′ = 3:7)¹⁾ | 20′ = 5:5) | | | | | |
| Comparative | [1-6, 2- | [1-9, 2- | 100% | 99% | 95% | 92% | 387 h |
| Example 28 | 19′] & | 20′] & | | | | | |
| | {G-1} | {G-7} | | | | | |
| | (1-6:2- | (1-9:2- | | | | | |
| | 19′ = 7:3) | 20′ = 5:5) | | | | | |
| Comparative | [1-6, 2- | [1-9, 2- | 100% | 100% | 96% | 94% | 412 h |
| Example 29 | 19′] & | 20′] & | | | | | |
| | {G-1} | {G-7} | | | | | |
| | (1-6:2- | (1-9:2- | | | | | |
| | 19′ = 5:5) | 20′ = 7:3) | | | | | |
| Comparative | [1-6, 2- | [1-9, 2- | 101% | 98% | 95% | 93% | 418 h |
| Example 30 | 19′] & | 20′] & | | | | | |
| | {G-1} | {G-7} | | | | | |
| | (1-6:2- | (1-9:2- | | | | | |
| | 19′ = 5:5) | 20′ = 3:7) | | | | | |

In Table 4, ratios in parentheses indicate weight ratios between hosts.

### Examples 7 to 11

Additional light-emitting devices were manufactured in substantially the same manner as in Comparative Example 9, except that Compounds 1-6 and 2-19 were utilized as hosts in forming a first emission layer and Compounds 1-9′ and 2-20 were utilized as hosts in forming a second emission layer.

### Examples 12 to 16

Additional light-emitting devices were manufactured in substantially the same manner as in Comparative Example 9, except that Compounds 1-6′ and 2-19 were utilized as hosts in forming a first emission layer and Compounds 1-9 and 2-20 were utilized as hosts in forming a second emission layer.

The results of Examples 7 to 16 are shown in Table 5.

TABLE 5

| | | | Driving | | | | |
|---|---|---|---|---|---|---|---|
| | | | voltage | | Efficiency | Efficiency | Lifespan |
| | | | @ 100 | Efficiency | @ 1000 | @ 10000 | @ 10000 |
| | | | nit | @ 100 nit | nit | nit | nit |
| | [Host] & {Dopant} | | (V) | (Cd/A) | (Cd/A) | (Cd/A) | (T95) |
| | EML1 | EML2 | | | | | |
| Example 7 | [1-6, 2-19] | [1-9′, 2- | 100% | 102% | 98% | 95% | 610 h |
| | & {G-1} | 20] & {G- | | | | | |
| | (1-6:2- | 7} | | | | | |
| | 19 = 3:7) ¹⁾ | (1-9:2- | | | | | |
| | | 20 = 5:5) | | | | | |
| Example 8 | [1-6, 2-19] | [1-9′, 2- | 100% | 99% | 95% | 93% | 520 h |
| | & {G-1] | 20] & {G- | | | | | |
| | (1-6:2- | 7} | | | | | |
| | 19 = 7:3) | (1-9:2- | | | | | |
| | | 20 = 5:5) | | | | | |
| Example 9 | [1-6, 2-19] | [1-9′, 2- | 100% | 100% | 98% | 96% | 510 h |
| | & {G-1] | 20] & {G- | | | | | |
| | (1-6:2- | 7} | | | | | |
| | 19 = 5:5) | (1-9:2- | | | | | |
| | | 20 = 5:5) | | | | | |
| Example 10 | [1-6, 2-19] | [1-9′, 2- | 100% | 100% | 97% | 95% | 520 h |
| | & {G-1] | 20] & {G- | | | | | |
| | (1-6:2- | 7} | | | | | |
| | 19 = 5:5) | (1-9:2- | | | | | |
| | | 20 = 7:3) | | | | | |
| Example 11 | [1-6, 2-19] | [1-9′, 2- | 101% | 98% | 95% | 93% | 510 h |
| | & {G-1] | 20] & {G- | | | | | |
| | (1-6:2- | 7} | | | | | |
| | 19 = 5:5) | (1-9:2- | | | | | |
| | | 20 = 3:7) | | | | | |
| Example 12 | [1-6′, 2-19] | [1-9, 2- | 100% | 102% | 98% | 95% | 620 h |
| | & {G-1} | 20] & {G- | | | | | |
| | (1-6′:2- | 7} | | | | | |
| | 19 = 3:7) | (1-9:2- | | | | | |
| | | 20 = 5:5) | | | | | |
| Example 13 | [1-6′, 2-19] | [1-9, 2- | 100% | 99% | 95% | 93% | 630 h |
| | & {G-1] | 20] & {G- | | | | | |
| | (1-6′:2- | 7} | | | | | |
| | 19 = 7:3) | (1-9:2- | | | | | |
| | | 20 = 5:5) | | | | | |
| Example 14 | [1-6′, 2-19] | [1-9, 2- | 100% | 100% | 98% | 96% | 612 h |
| | & {G-1] | 20] & {G- | | | | | |
| | (1-6′:2- | 7} | | | | | |
| | 19 = 5:5) | (1-9:2- | | | | | |
| | | 20 = 5:5) | | | | | |
| Example 15 | [1-6′, 2-19] | [1-9, 2- | 100% | 100% | 97% | 95% | 613 h |
| | & {G-1] | 20] & {G- | | | | | |
| | (1-6′:2- | 7} | | | | | |
| | 19 = 5:5) | (1-9:2- | | | | | |
| | | 20 = 7:3) | | | | | |
| Example 16 | [1-6′, 2-19] | [1-9, 2- | 101% | 98% | 95% | 93% | 615 h |
| | & {G-1] | 20] & {G- | | | | | |
| | (1-6′:2- | 7} | | | | | |
| | 19 = 5:5) | (1-9:2- | | | | | |
| | | 20 = 3:7) | | | | | |

In Table 5, ratios in parentheses indicate weight ratios between hosts.

Referring to Table 5, it was confirmed that light-emitting devices in which any one of the hole-transporting hosts in the first emission layer or the second emission layer included deuterium each exhibited excellent or suitable lifespan, compared to the light-emitting devices of Comparative Examples 9 to 12. It was also confirmed that the light-emitting devices of Examples 12 to 16, in which only the second emission layer included the hole-transporting host including deuterium, exhibited relatively improved lifespan results, compared to the light-emitting devices of Examples 7 to 11, in which only the first emission layer included the hole-transporting host including deuterium.

### Comparative Examples 31 to 42

A glass substrate with a 15 Ω/cm² (800 Å) ITO/Ag/ITO anode formed thereon (a product of Corning Inc.) was cut to a size of 50 mm×50 mm×0.7 mm, sonicated with isopropyl alcohol and pure water each for 5 minutes, and then cleaned by exposure to ultraviolet rays and ozone for 15 minutes. The resultant glass substrate was loaded onto a vacuum deposition apparatus.

HAT-CN was deposited on the ITO/Ag/ITO anode of the glass substrate to form a hole injection layer having a thickness of 50 Å, NPB was deposited on the hole injection layer to form a hole transport layer having a thickness of 600 Å, TCTA was deposited on the hole transport layer to form an electron blocking layer having a thickness of 50 Å, HT56 and FD23 were co-deposited at a weight ratio of 97:3 on the electron blocking layer to form a first blue emission layer having a thickness of 200 Å, T2T was deposited on the first blue emission layer to form a hole blocking layer having a thickness of 50 Å, and TPM-TAZ and LiQ were co-deposited at a weight ratio of 1:1 on the hole blocking layer to form an electron transport layer having a thickness of 200 Å.

Subsequently, BCP and Li were co-deposited at a weight ratio of 99:1 on the electron transport layer to form an n-type charge generation layer having a thickness of 150 Å, and HAT-CN was deposited on the n-type charge generation layer to form a p-type charge generation layer having a thickness of 50 Å.

Subsequently, NPB was deposited on the p-type charge generation layer to form a hole transport layer having a thickness of 500 Å, TCTA was deposited on the hole transport layer to form an electron blocking layer having a thickness of 50 Å, HT56 and FD23 were co-deposited at a weight ratio of 97:3 on the electron blocking layer to form a second blue emission layer having a thickness of 200 Å, T2T was deposited on the second blue emission layer to form a hole blocking layer having a thickness of 50 Å, and TPM-TAZ and LiQ were co-deposited at a weight ratio of 1:1 on the hole blocking layer to form an electron transport layer having a thickness of 200 Å.

Subsequently, BCP and Li were co-deposited at a weight ratio of 99:1 on the electron transport layer to form an n-type charge generation layer having a thickness of 150 Å, and HAT-CN was deposited on the n-type charge generation layer to form a p-type charge generation layer having a thickness of 50 Å.

Subsequently, NPB was deposited on the p-type charge generation layer to form a hole transport layer having a thickness of 400 Å, TCTA was deposited on the hole transport layer to form an electron blocking layer having a thickness of 50 Å, HT56 and FD23 were co-deposited at a weight ratio of 97:3 on the electron blocking layer to form a third blue emission layer having a thickness of 200 Å, T2T was deposited on the third blue emission layer to form a hole blocking layer having a thickness of 50 Å, and TPM-TAZ and LiQ were co-deposited at a weight ratio of 1:1 on the hole blocking layer to form an electron transport layer having a thickness of 200 Å.

Subsequently, BCP and Li were co-deposited at a weight ratio of 99:1 on the electron transport layer to form an n-type charge generation layer having a thickness of 150 Å, and HAT-CN was deposited on the n-type charge generation layer to form a p-type charge generation layer having a thickness of 50 Å.

Subsequently, NPB was deposited on the p-type charge generation layer to form a hole transport layer having a thickness of 600 Å, TCTA was deposited on the hole transport layer to form an electron blocking layer having a thickness of 50 Å, and emission layer materials of Table 1 were co-deposited on the electron blocking layer to form a green emission layer having a thickness of 200 Å (wherein, in case of a single-layered emission layer, a thickness was 200 Å (Comparative Examples 31 to 34), and in case of a double-layered emission layer, a thickness of each layer was 100 Å, and the stacking order was Green EML A and then Green EML B) (at a dopant doping concentration of 5 wt %) (Comparative Examples 35 to 42).

TPM-TAZ and LiQ were co-deposited at a weight ratio of 1:1 on the emission layer to form an electron transport layer having a thickness of 300 Å.

Subsequently, Yb was deposited on the electron transport layer to a thickness of 10 Å, and Ag and Mg were co-deposited at a weight ratio of 9:1 to form a cathode having a thickness of 100 Å. Then, CPL was vacuum-deposited on the cathode to form a capping layer having a thickness of 700 Å, thereby completing the manufacture of a tandem-type or kind light-emitting device.

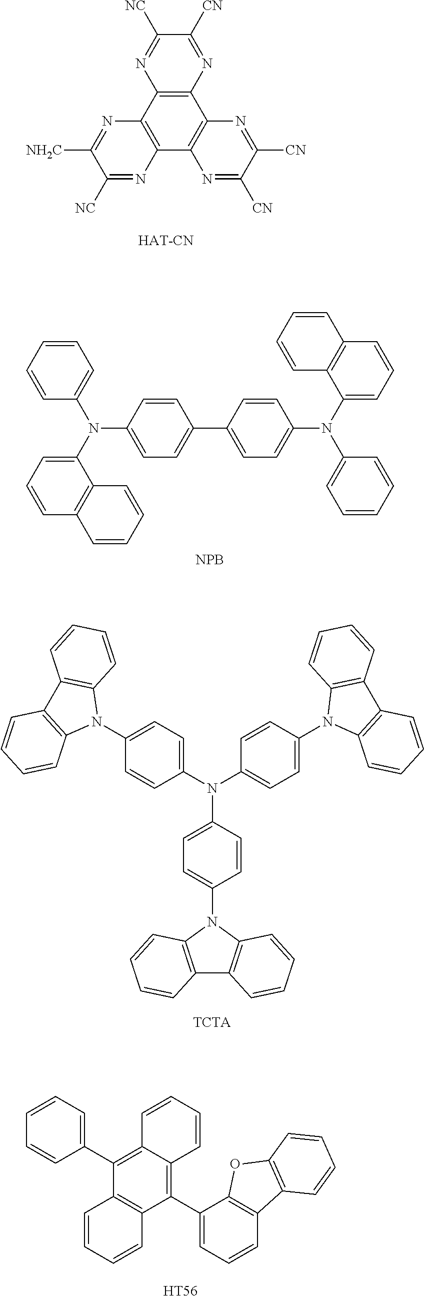

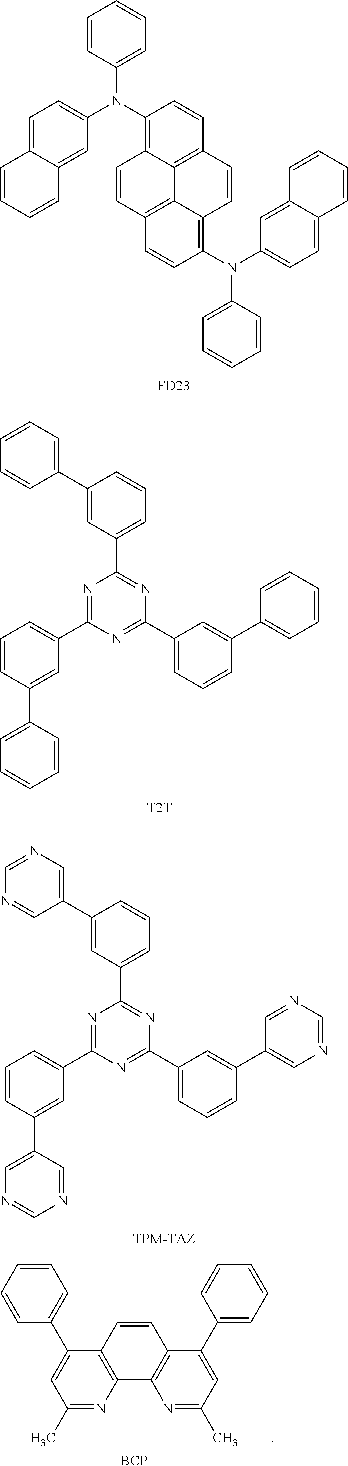

The results of Comparative Examples 31 to 42 are shown in Table 6.

TABLE 6

| | | | Driving | Efficiency | Efficiency | Efficiency | Lifespan |
|---|---|---|---|---|---|---|---|
| | | | voltage | @ 100 | @ 1000 | @ 10000 | @ 10000 |
| | | | @ 100 nit | nit | nit | nit | nit |
| | [Host] & {Dopant} | | (V) | (Cd/A) | (Cd/A) | (Cd/A) | (T95) |
| | Single-layered | | | | | | |
| | Green EML | | | | | | |
| Comparative | [1-6′, 2-19] & {G-1} | | 100% | 100% | 91% | 75% | 400 h |
| Example 31 | | | | | | | |
| Comparative | [1-9′, 2-20] & {G-7} | | 100% | 100% | 90% | 77% | 400 h |
| Example 32 | | | | | | | |
| Comparative | [1-6′, 2-19] & {G-7} | | 105% | 94% | 86% | 69% | 310 h |
| Example 33 | | | | | | | |
| Comparative | [1-9′, 2-20] & {G-1} | | 106% | 93% | 85% | 71% | 300 h |
| Example 34 | | | | | | | |
| | Green | Green | | | | | |
| | EML A | EML B | | | | | |
| Comparative | [1-6′, 2- | [1-9′, 2- | 100% | 100% | 99% | 97% | 470 h |
| Example 35 | 19] & {G- | 20] & | | | | | |
| | 1} | {G-7} | | | | | |
| Comparative | [1-9′, 2- | [1-6′, 2- | 105% | 100% | 89% | 75% | 280 h |
| Example 36 | 20] & {G- | 19] & | | | | | |
| | 7) | {G-1} | | | | | |
| Comparative | [1-6′, 2- | [1-9′, 2- | 105% | 94% | 92% | 90% | 440 h |
| Example 37 | 19] & {G- | 20] & | | | | | |
| | 7) | {G-1} | | | | | |
| Comparative | [1-9′, 2- | [1-6′, 2- | 106% | 93% | 84% | 73% | 250 h |
| Example 38 | 20] & {G- | 19] & | | | | | |
| | 1} | {G-7} | | | | | |
| Comparative | [1-6′, 2- | [1-9′, 2- | 100% | 100% | 98% | 96% | 470 h |
| Example 39 | 19] & {G- | 20] & | | | | | |
| | 1} | {G-7} | | | | | |
| | (1-6:2- | (1-9:2- | | | | | |
| | 19 = 3:7)¹⁾ | 20 = 5:5) | | | | | |
| Comparative | [1-6′, 2- | [1-9′, 2- | 101% | 100% | 94% | 90% | 480 h |
| Example 40 | 19] & {G- | 20] & | | | | | |
| | 1} | {G-7} | | | | | |
| | (1-6:2- | (1-9:2- | | | | | |
| | 19 = 7:3) | 20 = 5:5) | | | | | |
| Comparative | [1-6′, 2- | [1-9′, 2- | 101% | 100% | 97% | 95% | 500 h |
| Example 41 | 19] & {G- | 20] & | | | | | |
| | 1} | {G-7} | | | | | |
| | (1-6:2- | (1-9:2- | | | | | |
| | 19 = 5:5) | 20 = 7:3) | | | | | |
| Comparative | [1-6′, 2- | [1-9′, 2- | 102% | 100% | 96% | 93% | 490 h |
| Example 42 | 19] & {G- | 20] & | | | | | |
| | 1} | {G-7} | | | | | |
| | (1-6:2- | (1-9:2- | | | | | |
| | 19 = 5:5) | 20 = 3:7) | | | | | |

in Table 6, ratios in parentheses indicate weight ratios between hosts.

### Comparative Examples 43 to 48 and Examples 17 to 22

Additional light-emitting devices were manufactured in substantially the same manner as described in Table 5, except that, in forming an emission layer, Compound 1-6 was utilized instead of Compound 1-6′ and Compound 1-9 was utilized instead of Compound 1-9′.

The results of Comparative Examples 43 to 48 and Examples 17 to 22 are shown in Table 7.

TABLE 7

| | | | Driving | | | | |
|---|---|---|---|---|---|---|---|
| | | | voltage | Efficiency | Efficiency | Efficiency | Lifespan |
| | | | @ 100 | @ 100 | @ 1000 | @ 10000 | @ 10000 |
| | | | nit | nit | nit | nit | nit |
| | [Host] & {Dopant} | | (V) | (Cd/A) | (Cd/A) | (Cd/A) | (T95) |
| | Single-layered | | | | | | |
| | Green EML | | | | | | |
| Comparative | [1-6, 2-19] & {G-1} | | 100% | 100% | 90% | 73% | 410 h |
| Example 43 | | | | | | | |
| Comparative | [1-9, 2-20] & {G-7} | | 100% | 100% | 88% | 74% | 410 h |
| Example 44 | | | | | | | |
| Comparative | [1-6, 2-19] & {G-7} | | 105% | 94% | 85% | 67% | 330 h |
| Example 45 | | | | | | | |
| Comparative | [1-9, 2-20] & {G-1} | | 106% | 93% | 83% | 65% | 350 h |
| Example 46 | | | | | | | |
| | Green | Green | | | | | |
| | EML A | EML B | | | | | |
| Example 17 | [1-6, 2-19] | [1-9, 2- | 100% | 100% | 98% | 96% | 690 h |
| | & {G-1} | 20] & | | | | | |
| | | {G-7} | | | | | |
| Comparative | [1-9, 2-20] | [1-6, 2- | 105% | 100% | 88% | 73% | 295 h |
| Example 47 | & {G-7} | 19] & | | | | | |
| | | {G-1} | | | | | |
| Example18 | [1-6, 2-19] | [1-9, 2- | 105% | 95% | 93% | 91% | 670 h |
| | & {G-7} | 20] & | | | | | |
| | | {G-1} | | | | | |
| Comparative | [1-9, 2-20] | [1-6, 2- | 106% | 94% | 83% | 71% | 275 h |
| Example 48 | & {G-1} | 19] & | | | | | |
| | | {G-7} | | | | | |
| Example 19 | [1-6, 2-19] | [1-9, 2- | 100% | 100% | 98% | 96% | 670 h |
| | & {G-1} | 20] & | | | | | |
| | (1-6:2- | {G-7} | | | | | |
| | 19 = 3:7)¹⁾ | (1-9:2- | | | | | |
| | | 20 = 5:5) | | | | | |
| Example 20 | [1-6, 2-19] | [1-9, 2- | 101% | 100% | 95% | 91% | 680 h |
| | & {G-1} | 20] & | | | | | |
| | (1-6:2- | {G-7} | | | | | |
| | 19 = 7:3) | (1-9:2- | | | | | |
| | | 20 = 5:5) | | | | | |
| Example 21 | [1-6, 2-19] | [1-9, 2- | 101% | 100% | 96% | 94% | 710 h |
| | & {G-1} | 20] & | | | | | |
| | (1-6:2- | {G-7} | | | | | |
| | 19 = 5:5) | (1-9:2- | | | | | |
| | | 20 = 7:3) | | | | | |
| Example 22 | [1-6, 2-19] | [1-9, 2- | 102% | 100% | 96% | 92% | 690 h |
| | & {G-1} | 20] & | | | | | |
| | (1-6:2- | {G-7} | | | | | |
| | 19 = 5:5) | (1-9:2- | | | | | |
| | | 20 = 3:7) | | | | | |

In Table 7, ratios in parentheses indicate weight ratios between hosts.

The light-emitting devices of Comparative Examples 43 to 46 each included a hole-transporting host including deuterium, but only a single-layered emission layer. The light-emitting devices of Comparative Examples 47 and 48 had HOMO and LUMO energy relationships opposite that of the light-emitting devices of Examples 17 and 18, respectively (e.g., the compositions of EML A and EML B were switched or exchanged when Comparative Example 47 is compared with Example 17, and Comparative Example 48 is compared with Example 18).

When the light-emitting devices of Examples in Table 7 were compared with the corresponding light-emitting devices of Comparative Examples in Table 6, it was confirmed that each of the light-emitting devices of Examples in Table 7 had longer lifespans than each of the light-emitting devices of Comparative Examples in Table 6.

Compound G-1 had a maximum emission wavelength of 530 nm, and Compound G-7 had a maximum emission wavelength of 534 nm. Here, the coincidence rate between the emission spectrum of Compound G-1 and the emission spectrum of Compound G-7 was 89%.

As described above, according to the one more embodiments, a light-emitting device may exhibit improved efficiency and/or long lifespan, as compared with devices in the related art.

Terms such as “substantially,” “about,” and “˜” are used as terms of approximation and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

It should be understood that the embodiments described herein should be considered in a descriptive sense only and not for purposes of limitation. Descriptions of features or aspects within each embodiment should typically be considered as being available for other similar features or aspects in other embodiments. While one or more embodiments have been described with reference to the drawings, it will be understood by those of ordinary skill in the art that various suitable changes in form and details may be made therein without departing from the spirit and scope as defined by the following claims and equivalents thereof.

What is claimed is:

1. A light-emitting device comprising:
a first electrode;
a second electrode facing the first electrode; and
an interlayer between the first electrode and the second electrode, wherein
the interlayer comprises a first emission layer and a second emission layer,
the first emission layer comprises a first hole-transporting host (HTH1), a first electron-transporting host (ETH1), and a first phosphorescent dopant (G1),
the second emission layer comprises a second hole-transporting host (HTH2), a second electron-transporting host (ETH2), and a second phosphorescent dopant (G2),
an electron mobility of the ETH1 is faster than that of the ETH2,
a hole mobility of the HTH2 is faster than that of the HTH1, and
the HTH1 and/or the HTH2 comprises deuterium.

2. The light-emitting device of claim 1, wherein the HTH1 and/or the HTH2 further comprises hydrogen, and
a ratio of hydrogen to deuterium in the HTH1 and/or the HTH2 is in a range of about 1:9 to about 9:1.

3. The light-emitting device of claim 1, wherein the first emission layer and the second emission layer are in direct contact with each other.

4. The light-emitting device of claim 1, wherein the HTH1 and the HTH2 are different from each other, and
the ETH1 and the ETH2 are different from each other.

5. The light-emitting device of claim 1, wherein an absolute value of a lowest unoccupied molecular orbital (LUMO) energy level of the ETH1 is greater than an absolute value of a LUMO energy level of the ETH2.

6. The light-emitting device of claim 1, wherein the LUMO energy level of the ETH1 and the LUMO energy level of the ETH2 satisfy Condition (1):
0.01 eV≤|E_{LUMO_ETH2}−E_{LUMO_ETH1}|≤0.50 eV (1).

7. The light-emitting device of claim 1, wherein an absolute value of a highest occupied molecular orbital (HOMO) energy level of the HTH1 is greater than an absolute value of a HOMO energy level of the HTH2.

8. The light-emitting device of claim 1, wherein the HOMO energy level of the HTH1 and the HOMO energy level of the HTH2 satisfy Condition (2):
0.01 eV≤|E_{HOMO_HTH2}−E_{HOMO_HTH1}|≤0.50 eV (2).

9. The light-emitting device of claim 1, wherein the HTH1 and the HTH2 each independently comprise a compound represented by one of Formulae 311-1 to 311-6, and
ETH1 and the ETH2 each independently comprise a compound represented by one of Formulae 312-1 to 312-4 and 313: 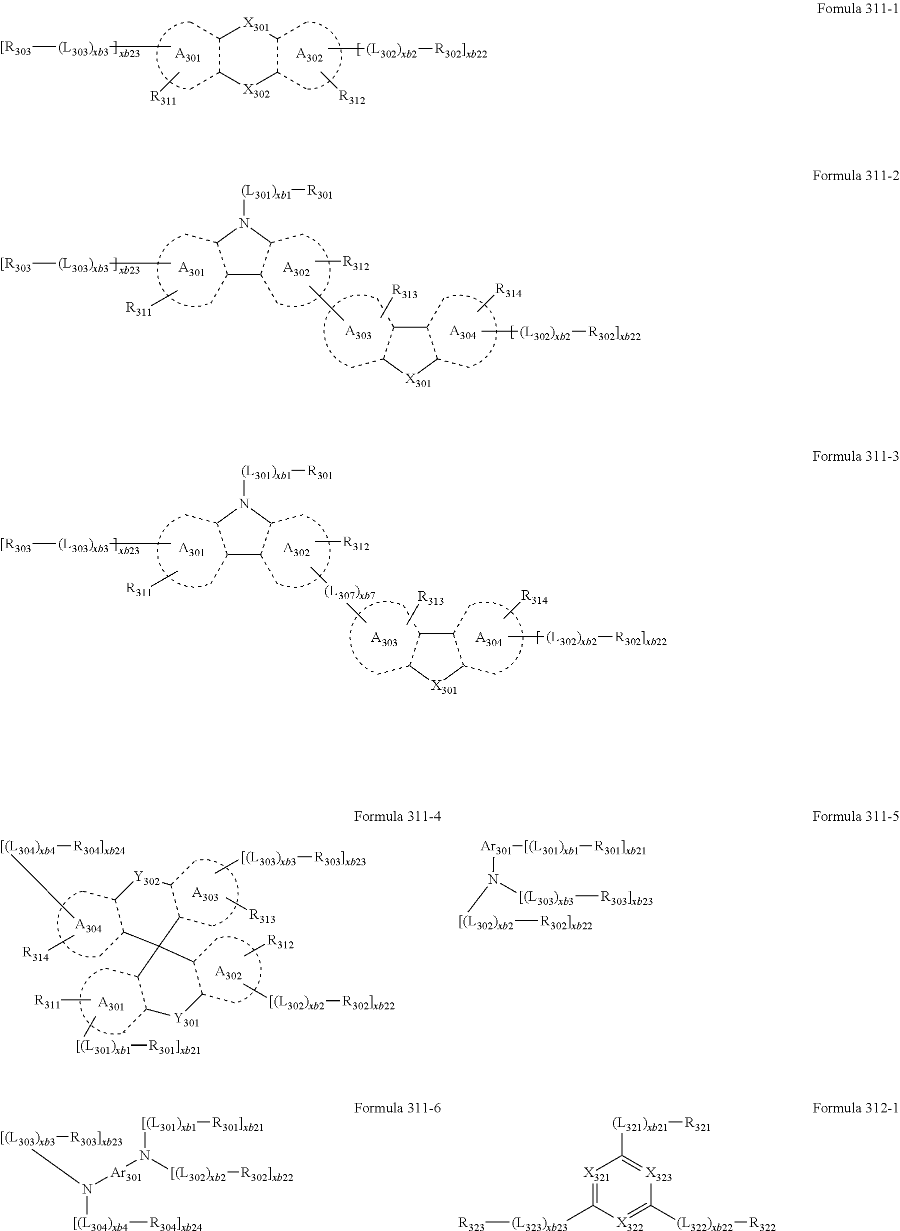 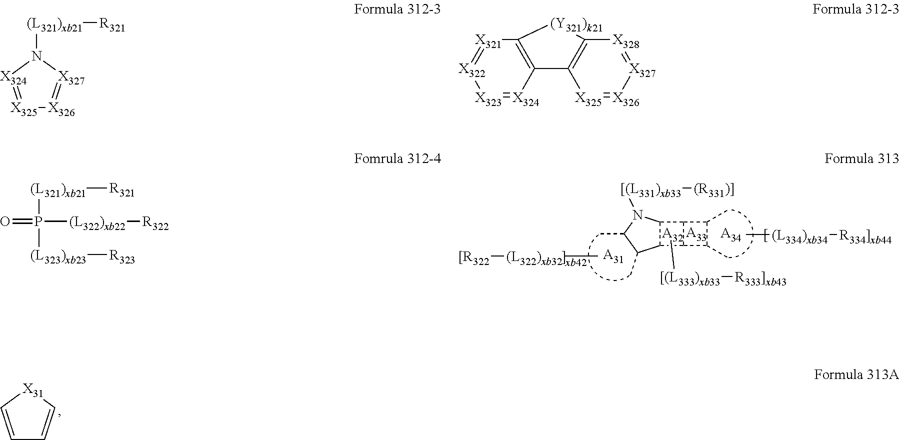
and
wherein, in Formulae 311-1 to 311-6, 312-1 to 312-4, 313, and 313 Å,
Ar₃₀₁ is a C₃-C₆₀ carbocyclic group unsubstituted or substituted with at least one R₁₀ₐ or a C₁-C₆₀ heterocyclic group unsubstituted or substituted with at least one R₁₀ₐ,
A₃₀₁ to A₃₀₄ are each independently a C₃-C₆₀ carbocyclic group or a C₁-C₆₀ heterocyclic group,
X₃₀₁ is O, S, N-[(L₃₀₄)_{xb4}-R₃₀₄], C[L₃₀₄)_{xb4}-R₃₀₄][(L₃₀₅)_{xb5}-R₃₀₅], or Si[(L₃₀₄)_{xb4}-R₃₀₄][(L₃₀₅)_{xb5}-R₃₀₅],
X₃₀₂, Y₃₀₁, and Y₃₀₂ are each independently a single bond, O, S, N-[(L₃₀₅)_{xb5}-R₃₀₅], C[(L₃₀₄)_{xb4}-R₃₀₄][(L₃₀₅)_{xb5}-R₃₀₅], Si[(L₃₀₄)_{xb4}-R₃₀₄][(L₃₀₅)_{xb5}-R₃₀₅], or S(═O)₂,
xb1 to xb5 are each independently 0, 1, 2, 3, 4, or 5,
xb6 is 1, 2, 3, 4, or 5,
X₃₂₁ to X₃₂₈ are each independently N or C[(L₃₂₄)_{xb24}-R₃₂₄],
Y₃₂₁ is *—O—*′, *—S-′, *—N[(L₃₂₅)_{xb25}-R₃₂₅]—*′, *—C[(L₃₂₅)_{xb25}-R₃₂₅][(L₃₂₆)_{xb26}-R₃₂₆]—*′, *—C[(L₃₂₅)_{xb25}-R₃₂₅]═C[(L₃₂₆)_{xb26}-R₃₂₆]—*′, *—C[(L₃₂₅)_{xb25}-R₃₂₅]═N—*′, or *—N═C[(L₃₂₆)_{xb26}-R₃₂₆]—*′,
k21 is 0, 1, or 2, wherein Y₃₂₁ does not exist when k21 is 0,
xb21 to xb26 are each independently 0, 1, 2, 3, 4, or 5,
A₃₁, A₃₂, and A₃₄ are each independently a C₃-C₆₀ carbocyclic group or a C₁-C₃₀ heterocyclic group,
A₃₃ is a group represented by Formula 313 Å,
X₃₁ is N[(L₃₃₅)_{xb35}-(R₃₃₅)], O, S, Se, C[(L₃₃₅)_{xb35}-(R₃₃₅)][(L₃₃₆)_{xb36}-(R₃₃₆)], or Si[(L₃₃₅)_{xb35}-(R₃₃₅)][(L₃₃₆)_{xb36}-(R₃₃₆)],
xb31 to xb36 are each independently 0, 1, 2, 3, 4, or 5,
xb42 to xb44 are each independently 0, 1, 2, 3, 4, 5, 6, 7, 8, 9, or 10,
L₃₀₁ to L₃₀₆, L₃₂₁ to L₃₂₆, and L₃₃₁ to L₃₃₆ are each independently a single bond, a C₁-C₂₀ alkylene group unsubstituted or substituted with at least one R₁₀ₐ, a C₁-C₂₀ alkenylene group unsubstituted or substituted with at least one R₁₀ₐ, a C₁-C₂₀ alkynylene group unsubstituted or substituted with at least one R₁₀ₐ, a C₃-C₁₀ cycloalkylene group unsubstituted or substituted with at least one R₁₀ₐ, a C₁-C₁₀ heterocycloalkylene group unsubstituted or substituted with at least one R₁₀ₐ, a C₃-C₁₀ cycloalkenylene group unsubstituted or substituted with at least one R₁₀ₐ, a C₁-C₁₀ heterocycloalkenylene group unsubstituted or substituted with at least one R₁₀ₐ, a C₆-C₆₀ arylene group unsubstituted or substituted with at least one R₁₀ₐ, a C₁-C₆₀ heteroarylene group unsubstituted or substituted with at least one R₁₀ₐ, a divalent non-aromatic condensed polycyclic group unsubstituted or substituted with at least one R₁₀ₐ, or a divalent non-aromatic condensed heteropolycyclic group unsubstituted or substituted with at least one R₁₀ₐ,
R₃₀₁ to R₃₀₅, R₃₁₁ to R₃₁₄, R₃₂₁ to R₃₂₆, and R₃₃₁ to R₃₃₆ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
two or more neighboring substituents of R₃₀₃, R₃₀₄, R₃₁₁, and R₃₂₁ to R₃₂₄ are optionally bonded to each other to form a C₃-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

10. The light-emitting device of claim 1, wherein the HTH1 and the HTH2 each independently comprise a compound selected from Compounds 1-1 to 1-14:

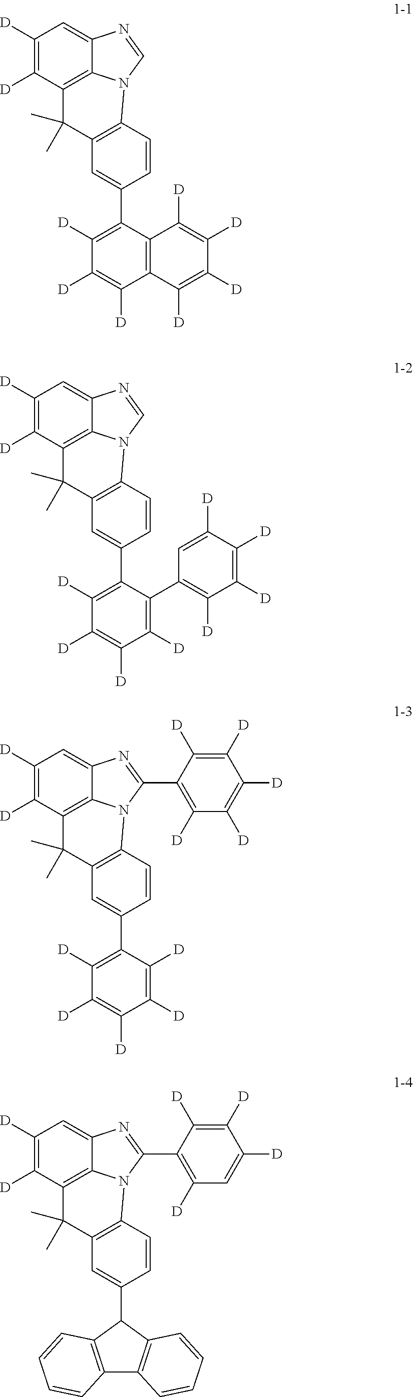 [SMILES: [2H]c1cc2ncn3c2c(c1[2H])C(C)(C)c1cc(-c2c([2H])c([2H])c([2H])c4c([2H])c([2H])c([2H])c([2H])c24)ccc1-3 ; [2H]c1cc(-c2c([2H])c([2H])c([2H])c([2H])c2-c2ccc3c(c2)C(C)(C)c2c([2H])c([2H])cc4ncn-3c24)c([2H])c([2H])c1[2H] ; [2H]c1cc2nc(-c3c([2H])c([2H])c([2H])c([2H])c3[2H])n3c2c(c1[2H])C(C)(C)c1cc(-c2c([2H])c([2H])c([2H])c([2H])c2[2H])ccc1-3 ; [2H]c1cc([2H])c(-c2nc3cc([2H])c([2H])c4c3n2-c2ccc(C3c5ccccc5-c5ccccc53)cc2C4(C)C)c([2H])c1[2H]]

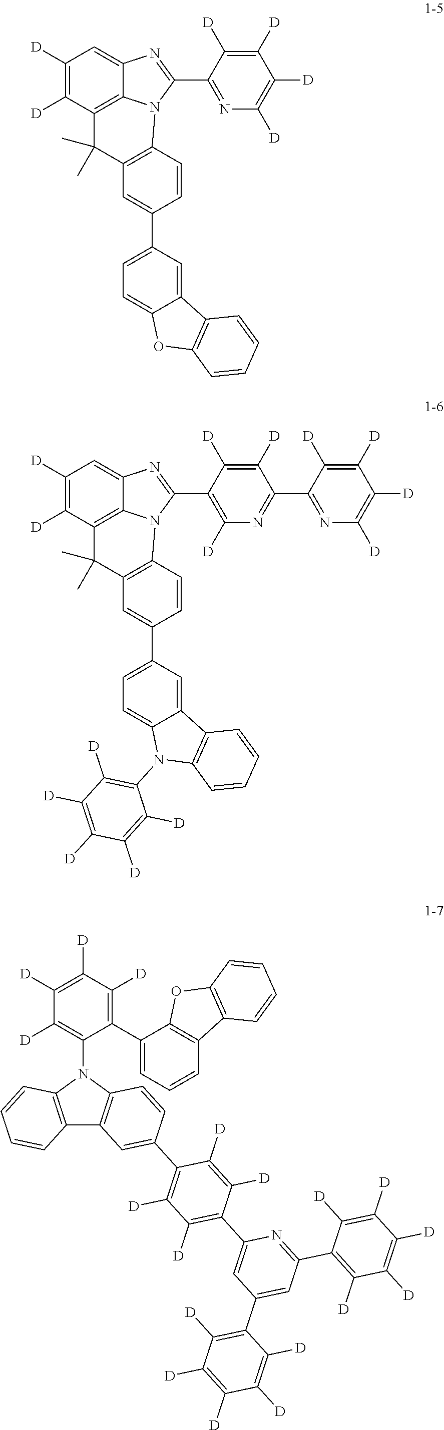 [SMILES: [2H]c1cc2nc(-c3nc([2H])c([2H])c([2H])c3[2H])n3c2c(c1[2H])C(C)(C)c1cc(-c2ccc4oc5ccccc5c4c2)ccc1-3 ; [2H]c1cc2nc(-c3c([2H])nc(-c4nc([2H])c([2H])c([2H])c4[2H])c([2H])c3[2H])n3c2c(c1[2H])C(C)(C)c1cc(-c2ccc4c(c2)c2ccccc2n4-c2c([2H])c([2H])c([2H])c([2H])c2[2H])ccc1-3 ; [2H]c1c([2H])c([2H])c(-c2cc(-c3c([2H])c([2H])c([2H])c([2H])c3[2H])nc(-c3c([2H])c([2H])c(-c4ccc5c(c4)c4ccccc4n5-c4c([2H])c([2H])c([2H])c([2H])c4-c4cccc5c4oc4ccccc45)c([2H])c3[2H])c2)c([2H])c1[2H]]

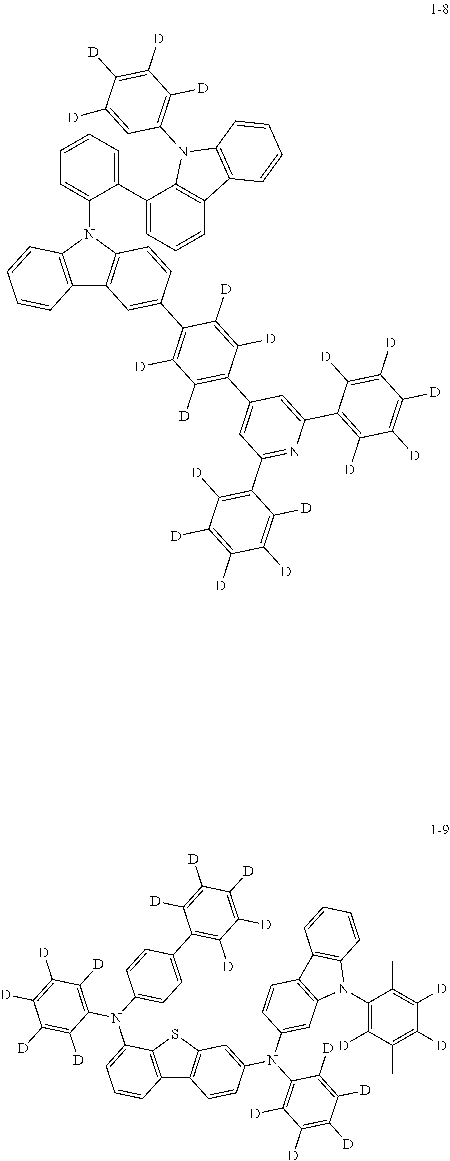 [SMILES: [2H]c1cc(-n2c3ccccc3c3cccc(-c4ccccc4-n4c5ccccc5c5cc(-c6c([2H])c([2H])c(-c7cc(-c8c([2H])c([2H])c([2H])c([2H])c8[2H])nc(-c8c([2H])c([2H])c([2H])c([2H])c8[2H])c7)c([2H])c6[2H])ccc54)c32)c([2H])c([2H])c1[2H] ; [2H]c1c([2H])c([2H])c(-c2ccc(N(c3c([2H])c([2H])c([2H])c([2H])c3[2H])c3cccc4c3sc3cc(N(c5ccc6c7ccccc7n(-c7c([2H])c(C)c([2H])c([2H])c7C)c6c5)c5c([2H])c([2H])c([2H])c([2H])c5[2H])ccc34)cc2)c([2H])c1[2H]]

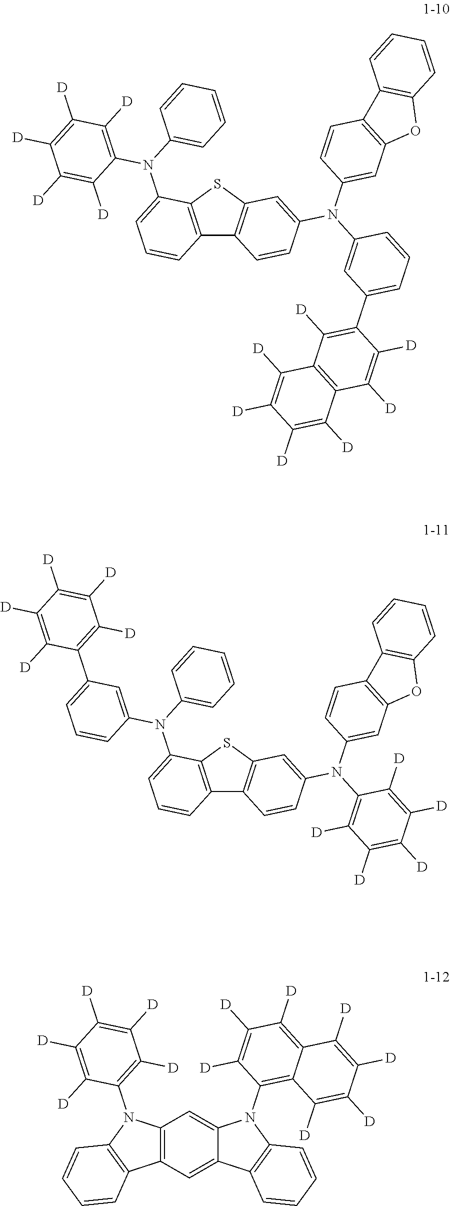 [SMILES: [2H]c1c([2H])c([2H])c(N(c2ccccc2)c2cccc3c2sc2cc(N(c4cccc(-c5c([2H])c([2H])c6c([2H])c([2H])c([2H])c([2H])c6c5[2H])c4)c4ccc5c(c4)oc4ccccc45)ccc23)c([2H])c1[2H] ; [2H]c1c([2H])c([2H])c(-c2cccc(N(c3ccccc3)c3cccc4c3sc3cc(N(c5ccc6c(c5)oc5ccccc56)c5c([2H])c([2H])c([2H])c([2H])c5[2H])ccc34)c2)c([2H])c1[2H] ; [2H]c1c([2H])c([2H])c(-n2c3ccccc3c3cc4c5ccccc5n(-c5c([2H])c([2H])c([2H])c6c([2H])c([2H])c([2H])c([2H])c56)c4cc32)c([2H])c1[2H]]

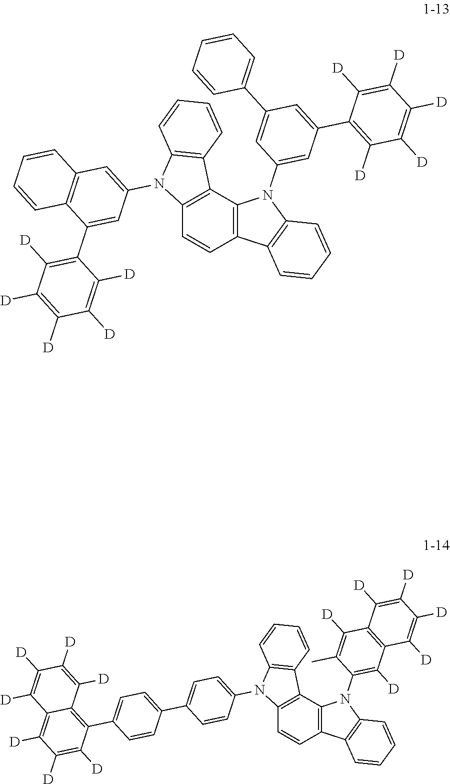 [SMILES: [2H]c1c([2H])c([2H])c(-c2cc(-c3ccccc3)cc(-n3c4ccccc4c4ccc5c(c6ccccc6n5-c5cc(-c6c([2H])c([2H])c([2H])c([2H])c6[2H])c6ccccc6c5)c43)c2)c([2H])c1[2H] ; [2H]c1c([2H])c([2H])c2c([2H])c(-n3c4ccccc4c4ccc5c(c6ccccc6n5-c5ccc(-c6ccc(-c7c([2H])c([2H])c([2H])c8c([2H])c([2H])c([2H])c([2H])c78)cc6)cc5)c43)c(C)c([2H])c2c1[2H]]

11. The light-emitting device of claim 1, wherein the ETH1 and the ETH2 each independently comprise a compound selected from Compounds 2-1 to 2-20:

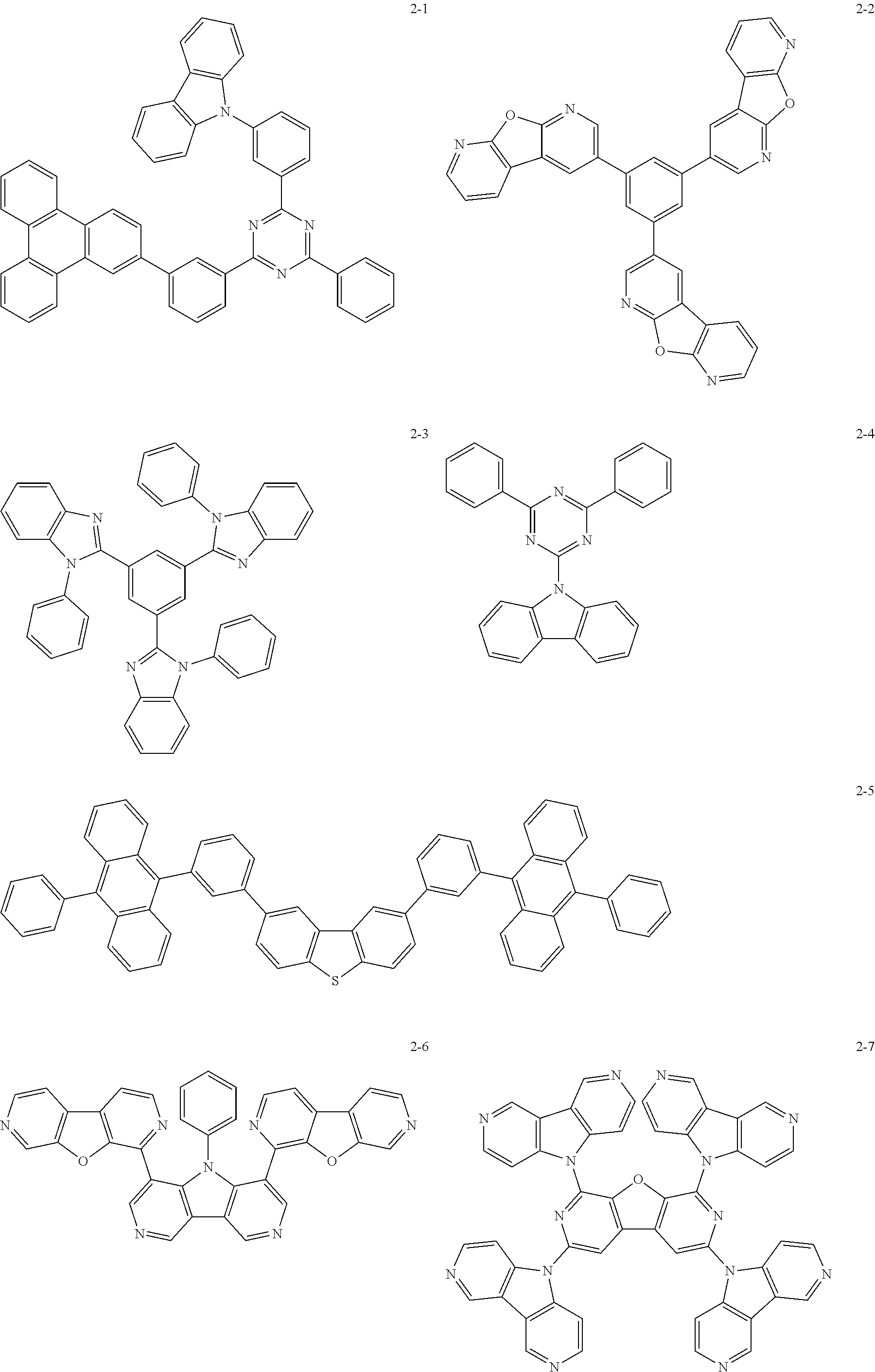

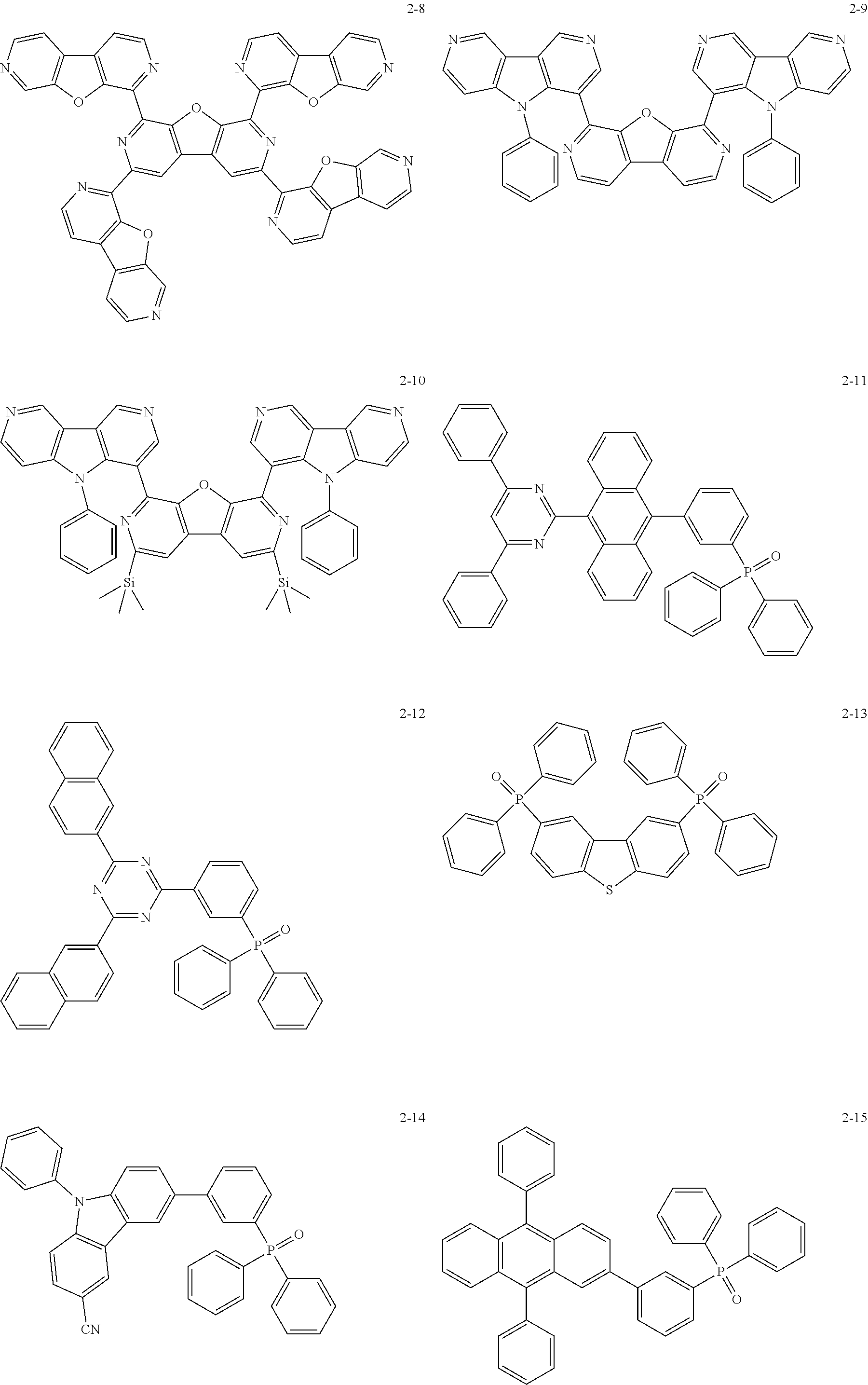

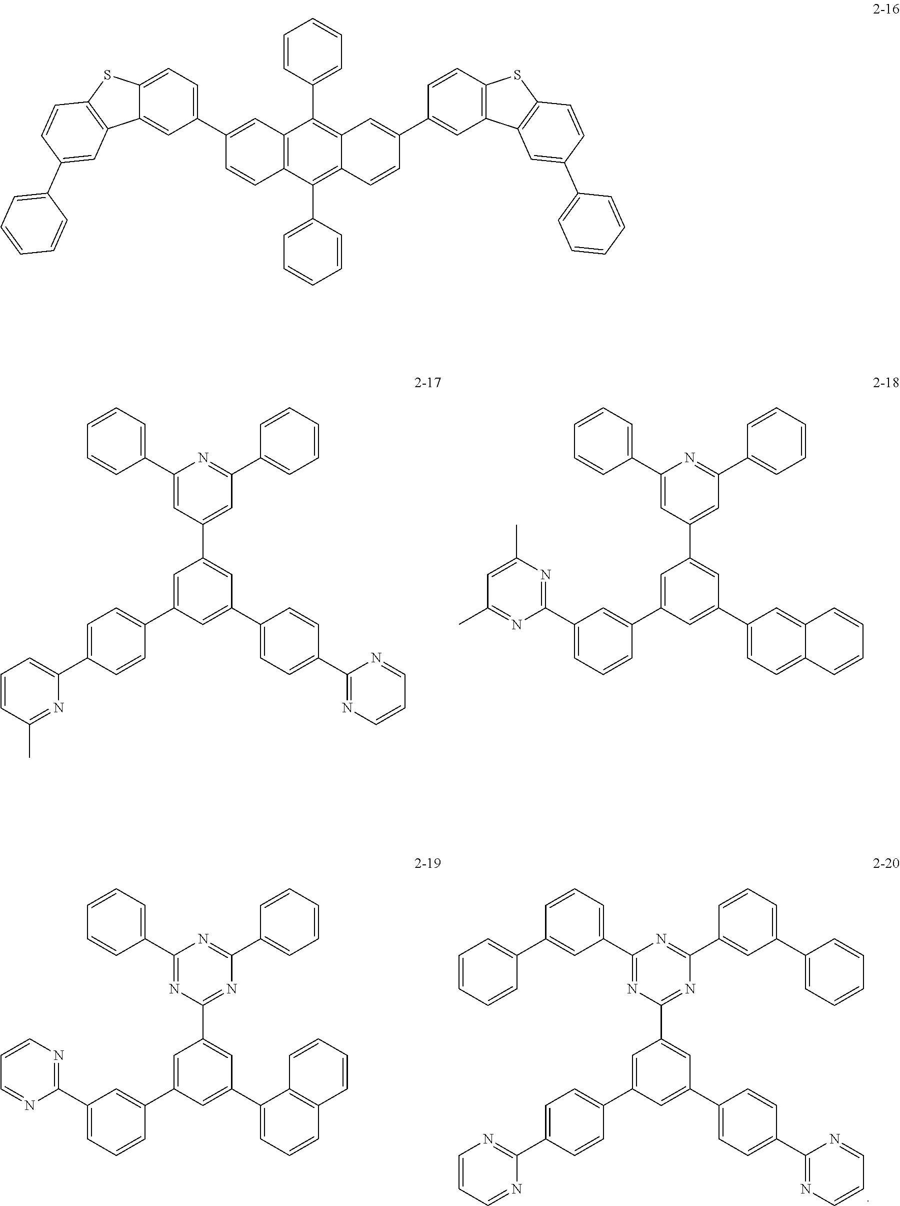 [SMILES: c1ccc(-c2ccc3sc4ccc(-c5ccc6c(-c7ccccc7)c7ccc(-c8ccc9sc%10ccc(-c%11ccccc%11)cc%10c9c8)cc7c(-c7ccccc7)c6c5)cc4c3c2)cc1 ; Cc1cccc(-c2ccc(-c3cc(-c4ccc(-c5ncccn5)cc4)cc(-c4cc(-c5ccccc5)nc(-c5ccccc5)c4)c3)cc2)n1 ; Cc1cc(C)nc(-c2cccc(-c3cc(-c4cc(-c5ccccc5)nc(-c5ccccc5)c4)cc(-c4ccc5ccccc5c4)c3)c2)n1 ; c1ccc(-c2nc(-c3ccccc3)nc(-c3cc(-c4cccc(-c5ncccn5)c4)cc(-c4cccc5ccccc45)c3)n2)cc1 ; c1ccc(-c2cccc(-c3nc(-c4cccc(-c5ccccc5)c4)nc(-c4cc(-c5ccc(-c6ncccn6)cc5)cc(-c5ccc(-c6ncccn6)cc5)c4)n3)c2)cc1]

12. The light-emitting device of claim 1, wherein the G1 and the G2 are identical to or different from each other.

13. The light-emitting device of claim 1, wherein the G1 and the G2 each independently have a maximum emission wavelength in a range of about 490 nm to about 560 nm.

14. The light-emitting device of claim 1, wherein a difference between a maximum emission wavelength of the G1 and a maximum emission wavelength of the G2 is about 10 nm or less.

15. The light-emitting device of claim 1, wherein a coincidence ratio between an emission spectrum of the G1 and an emission spectrum of the G2 is about 80% or more.

16. The light-emitting device of claim 1, wherein the G1 and the G2 are each independently a compound represented by Formula 411 or 412: 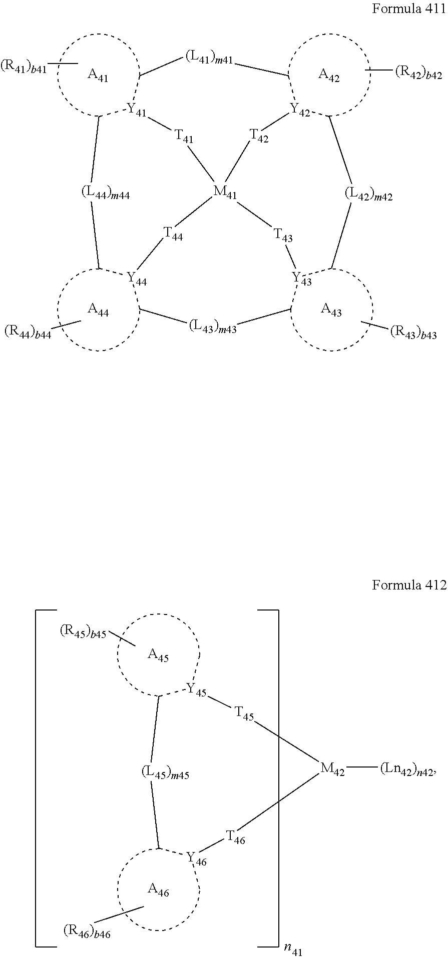
and
wherein, in Formulae 411 and 412,
M₄₁ is platinum (Pt), palladium (Pd), copper (Cu), silver (Ag), gold (Au), rhodium (Rh), iridium (Ir), ruthenium (Ru), osmium (Os), titanium (Ti), zirconium (Zr), hafnium (Hf), europium (Eu), terbium (Tb), or thulium (Tm),
n41 is 1, 2, or 3,
Ln₄₂ is an organic ligand, wherein n42 is 0, 1, or 2,
Y₄₁ to Y₄₆ are each independently N or C,
A₄₁ to A₄₆ are each independently a C₃-C₆₀ carbocyclic group or a C₁-C₆₀ heterocyclic group,
T41 to T46 are each independently a single bond, *—O—*′, or *—S—*′,
L₄₁ to L₄₅ are each independently a single bond, *—O—*′, *—S—*′, *—C(R₄₇)(R₄₈)—*′, *—C(R₄₇)=*′, *═C(R₄₇)—*′, *—C(R₄₇)═C(R₄₈)—*′, *—C(═O)—*′, *—C(═S)—*′, *—C≡C—*′, *—B(R₄₇)—*′, *—N(R₄₇)—*′, *—P(R₄₇)—*′, *—Si(R₄₇)(R₄₈)—*′, *—P(═O)(R₄₇)—*′, or *—Ge(R₄₇)(R₄₈)—*′,
m41 to m45 are each independently 0, 1, 2, or 3,
R₄₁ to R₄₈ are each independently hydrogen, deuterium, —F, —Cl, —Br, —I, a hydroxyl group, a cyano group, a nitro group, an amidino group, a hydrazino group, a hydrazono group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Bi(Q₄₁)(Q₄₂)(Q₄₃), —N(Q₄₁)(Q₄₂), —B(Q₄₁)(Q₄₂), —C(═O)(Q₄₁), —S(═O)₂(Q₄₁), or —P(═O)(Q₄₁)(Q₄₂),
R₄₇ and R₄₁; R₄₇ and R₄₂; R₄₇ and R₄₃; or R₄₇ and R₄₄ are optionally linked each other to form a C₃-C₆₀ carbocyclic group unsubstituted or substituted with at least one R₁₀ₐ or a C₁-C₆₀ heterocyclic group unsubstituted or substituted with at least one R₁₀ₐ,
b41 to b46 are each independently 1, 2, 3, 4, 5, 6, 7, or 8,
and *′ each indicate a binding site to a neighboring atom,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁₁ to Q₁₃, Q₂₁ to Q₂₃, Q₃₁ to Q₃₃, and Q₄₁ to Q₄₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

17. The light-emitting device of claim 1, wherein the G1 and the G2 are each independently selected from Compounds G-1 to G-12:

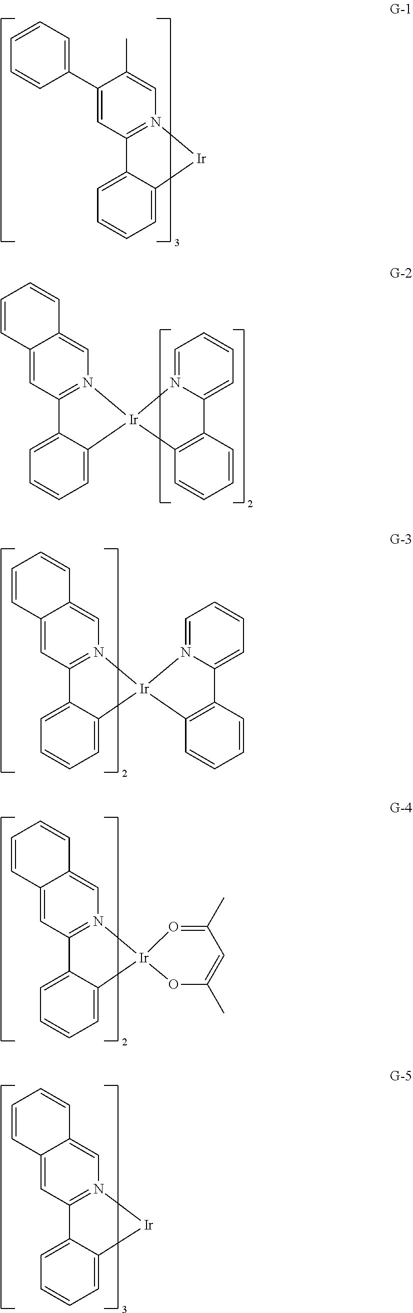

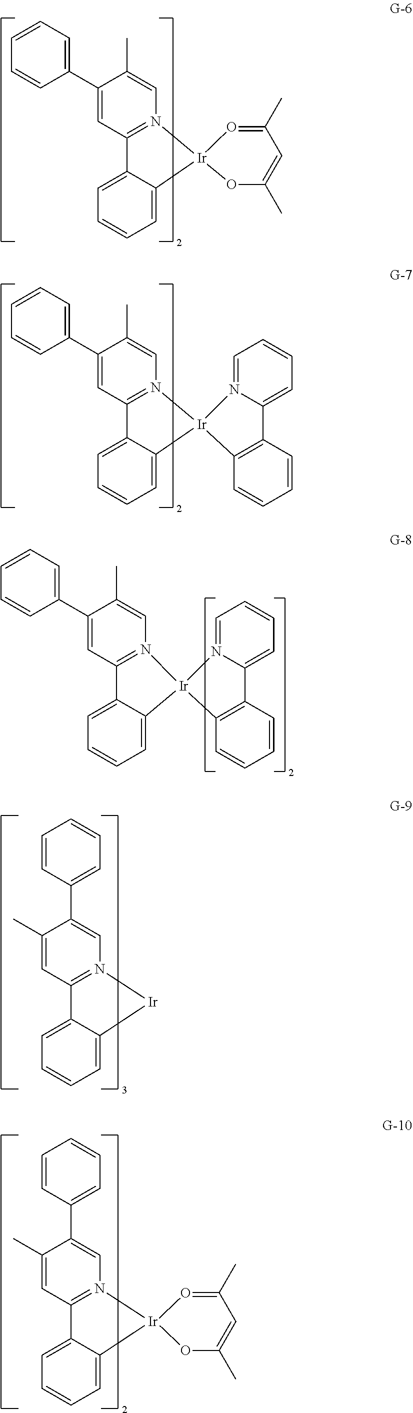

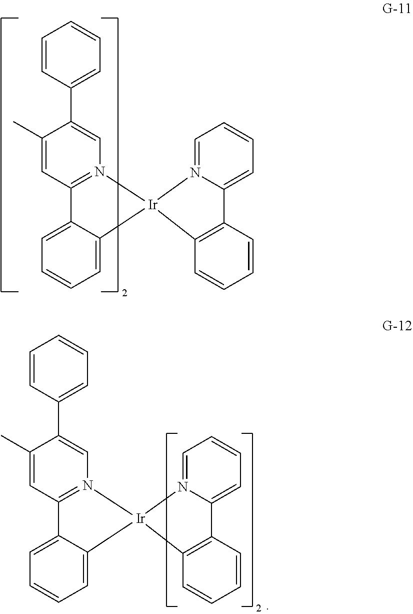

18. An electronic apparatus comprising the light-emitting device of claim 1.

19. The electronic apparatus of claim 18, further comprising quantum dots.

20. The electronic apparatus of claim 18, further comprising a thin-film transistor, wherein:
the thin-film transistor comprises a source electrode and a drain electrode, and
the first electrode of the light-emitting device is electrically connected to the source electrode or the drain electrode.
